(19) United States
(12) Patent Application Publication
(10) Pub. No.: US 2016/0214956 A1
(43) Pub. Date: Jul. 28, 2016

(54) NOVEL SOLUBLE GUANYLATE CYCLASE ACTIVATORS AND THEIR USE

(71) Applicant: GLAXOSMITHKLINE INTELLECTUAL PROPERTY DEVELOPMENT LIMITED, Brentford, Middlesex (GB)

(72) Inventors: Krista B. GOODMAN, Stevenage, Hertfordshire (GB); Achim Hans-Peter KRAUSS, King of Prussia (PA); Anne-Charlotte LE MONNIER DE GOUVILLE, Les Ulis (FR); Nerina DODIC, Les Ulis (FR)

(73) Assignee: GlaxoSmithKline Intellectual Property Development Limited, Brentford, Middlesex (GB)

(21) Appl. No.: 14/917,033

(22) PCT Filed: Sep. 5, 2014
(86) PCT No.: PCT/IB14/64291

§ 371 Date: Mar. 7, 2016

Related U.S. Application Data
(60) Provisional application No. 61/873,905, filed on Sep. 5, 2013.

(51) Int. Cl. C07D 401/04; C07D 401/14

(52) U.S. Cl. CPC C07D 401/04; C07D 401/14

(57) ABSTRACT

The invention relates to activators of soluble guanylate cyclase and their use in pharmaceutical compositions, primarily topically administered ophthalmic compositions. The pharmaceutical compositions are useful for reducing intraocular pressure in animals of the mammalian species.

### FIELD OF THE INVENTION

The invention relates to activators of soluble guanylate cyclase (sGC), pharmaceutically acceptable salts thereof, pharmaceutical compositions, processes for their preparation and their use in medicine, primarily topically administered ophthalmic compositions. The pharmaceutical compositions are useful for reducing intraocular pressure (IOP) in animals of the mammalian species. The present invention also relates to administering such pharmaceutical compositions to animals of the mammalian species, including humans, for reducing IOP, including elevated IOP caused by glaucoma or ocular hypertension.

### BACKGROUND OF THE INVENTION

Glaucoma is an optic neuropathy resulting in irreversible loss of visual function over time. Glaucoma is considered the second leading cause of blindness in the world. Predictions are for approximately 80 million people afflicted with glaucoma worldwide by 2020 (Quigley and Broman, Br J Ophthalmol 2006). Frequently, but not always, glaucoma is associated with elevated IOP which is recognized as an important risk factor for the disease. Ocular hypertension, a condition associated with elevated IOP that has not yet progressed to causing irreversible glaucomatous damage, is believed to represent the earliest stage of glaucoma. Therapeutic agents devised for the treatment of glaucoma and ocular hypertension have been designed to lower IOP, which remains the sole, proven treatable risk factor of the disease.

The drugs currently used for the treatment of glaucoma and ocular hypertension include prostaglandin analogs (e.g., latanoprost, bimatoprost, travoprost, tafluprost), beta-adrenergic blockers (e.g., timolol, betaxolol, levobunolol), alpha-adrenergic agonists (e.g., brimonidine, paraamino-clonidine), parasympathomimetics (e.g. pilocarpine, carbachol, acethylcholineesterase inhibitors), sympathomimetics (e.g., epinephrine, dipivalyl-epinephrine), carbonic anhydrase inhibitors (e.g., dorzolamide, brinzolamide). Pressure in the eye (IOP) is determined by the balance of aqueous humor production and aqueous humor outflow. It is generally accepted that elevated IOP is the result of compromised aqueous humor outflow. Thus, compounds that increase the outflow of aqueous humor are considered preferable for reducing IOP in glaucoma and ocular hypertensive patients. Prostaglandin analogs, sympathomimetics and parasympathomimetics are believed to decrease IOP by increasing aqueous outflow, whereas beta-blockers, alpha-adrenergic agonists and carbonic anhydrase inhibitors are believed to decrease IOP by reducing aqueous humor production. Prostaglandin analogs cause undesirable effects, such as increased conjunctival hyperaemia and iris hyperpigmentation, for example. Parasympathomimetics induce undesirable accommodative changes leading to blurring of vision. Sympathomimetics can also stimulate aqueous humor production which partially counteracts their effect on aqueous humor outflow and thus limits their resultant effect on IOP regulation. Some antiglaucoma drugs, e.g., timolol, produce systemic effects. These adverse events can lead to poor patient compliance and may necessitate withdrawal of drug therapy.

As a consequence, a need still exists to identify and develop anti-glaucoma drugs that specifically enhance aqueous humor drainage from the eye and, preferably, have a more limited adverse event profile.

Of the two primary aqueous humor outflow pathways in the eye, the conventional/trabecular outflow pathway represents the more attractive target since it is the site of outflow obstruction that leads to ocular hypertension. As reviewed by Ellis (Cell Physiol Biochem 2011) nitric oxide donors and guanylate cyclase activators have been shown to decrease IOP in humans, rabbits and monkeys. Nitric oxide is an endogenous activator of the soluble guanylate cyclase enzyme which in turn catalyzes the generation of cyclic GMP as a second messenger molecule. The role of the nitric oxide-soluble guanylate cyclase-cyclic GMP pathway in IOP regulation is well established (Ellis, Cell Physiol Biochem 2011). Components of this pathway, such as endothelial and neuronal type nitric oxide synthases responsible for the endogenous generation of nitric oxide, are present in the outflow pathway tissues. Thus, stimulation of sGC represents a novel ocular anti-hypertensive approach, regardless of whether the reduction in IOP through enhancement of aqueous humor drainage is caused by modulation of cell volume of trabecular meshwork or Schlemms Canal cells (Ellis, Cell Physiol Biochem 2011) or trabecular meshwork contractility (Stumpff and Wiederholt, Ophthalmologica 2000). Modulation of cell volume and/or contractility of structures in the trabecular outflow pathway had been proposed as mechanistic rationales for IOP regulation.

In U.S. Pat. No. 5,652,236, a method for reducing IOP in the mammalian eye by administration of guanylate cyclase inhibitors is claimed. In that context, it was surprising that guanylate cyclase activators were found to also reduce IOP.

### SUMMARY OF THE INVENTION

The present invention relates to novel compounds which are 2-pyridine pyrazole carboxylic acid or ester activators of sGC. Specifically, the invention is directed to compounds of formula (I), and pharmaceutically acceptable salts thereof:

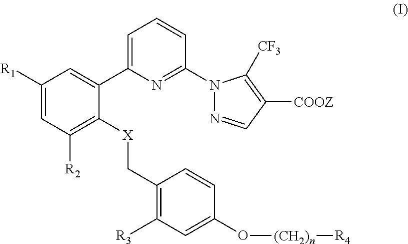

wherein:
R1 and R2 are each independently selected from H and halogen (suitably Cl, F, Br, I; preferably Cl, F);
R3 is selected from H, —CH₃ and F;
R4 is selected from —CF₃, —OCH₃, —CN, —COOH, morpholine, 3-(trifluoromethyl)-1-pyrazolyl, an optionally substituted 5- to 6-membered heteroaryl ring, wherein the optional substituents are independently —CN or —OCH₃, and an optionally substituted 5- to 6-membered heterocyclic ring;
X is selected from O and CH₂;
Z is selected from H and C₁₋₄ alkyl; and
n is 2 or 3.

The compounds of the invention are activators of sGC. Therefore, the present invention is directed to a method for activating sGC which method comprises contacting a cell with a compound of Formula (I), or a pharmaceutically acceptable salt thereof. The invention is still further directed to a method of activating sGC activity and treatment of disorders associated therewith using a compound of the invention or a pharmaceutical composition comprising a compound of the invention.

In one embodiment, the invention is directed to a method of treating an sGC-mediated disease or disorder which comprises administering a therapeutically effective amount of a compound according to Formula (I), or a pharmaceutically acceptable salt thereof, to a patient (a human or other mammal, particularly, a human) in need thereof. Such sGC-mediated diseases or disorders include diseases or disorders associated with poor aqueous humor drainage or elevated intraocular pressure. Such diseases or disorders include, but are not limited to, glaucoma and ocular hypertension.

In one embodiment, the invention is directed to a pharmaceutical composition comprising a compound of the invention according to Formula (I), or a pharmaceutically acceptable salt thereof, and a pharmaceutically acceptable excipient. Particularly, this invention is directed to a pharmaceutical composition for the treatment of an sGC-mediated disease or disorder, wherein the composition comprises a compound according to Formula (I), or a pharmaceutically acceptable salt thereof, and a pharmaceutically acceptable excipient.

In one embodiment, the invention is directed to a method of treating an ocular disorder caused by intraocular pressure comprising administering a safe and effective amount of a compound of Formula (I), or a pharmaceutically acceptable salt thereof, to a mammal in need thereof. Still yet further, the invention is directed to a method for reducing intraocular pressure in a mammal comprising administering a safe and effective amount of a compound of Formula (I), or a pharmaceutically acceptable salt thereof, to a mammal in need thereof. Still further, the invention is directed to a method of treating glaucoma comprising administering a safe and effective amount of a compound of Formula (I), or a pharmaceutically acceptable salt thereof, to a mammal in need thereof. Yet further, the invention is directed to a method of treating ocular hypertension comprising administering a safe and effective amount of a compound of Formula (I), or a pharmaceutically acceptable salt thereof, to a mammal in need thereof. As used herein, the term “mammal” includes, but is not limited to, humans.

In one embodiment, the invention is directed to a compound described herein, or a pharmaceutically acceptable salt thereof, for use in therapy. This invention provides a compound of Formula (I), or a pharmaceutically acceptable salt thereof, for use in therapy, specifically for use in the treatment of intraocular pressure, including, but not limited to glaucoma or ocular hypertension. Specifically, this invention provides a compound of Formula (I), or a pharmaceutically acceptable salt thereof, for use in therapy.

In one embodiment, the invention is directed to a compound described herein, or a pharmaceutically acceptable salt thereof, for use in the treatment of an ocular disease or disorder. This invention provides a compound of the invention for use in the treatment of an ocular disease or disorder, specifically, a disease or disorder recited herein. This invention provides a compound of Formula (I), or a pharmaceutically acceptable salt thereof, for use in the treatment of an ocular disorder.

In one embodiment, the invention is directed to the use of a compound of Formula (I), or a pharmaceutically acceptable salt thereof, as an active therapeutic substance. More specifically, this invention provides for the use of the compounds described herein for the treatment of an ocular disease or disorder, specifically, a disease or disorder recited herein. Accordingly, the invention provides for the use of a compound of Formula (I), or a pharmaceutically acceptable salt thereof, as an active therapeutic substance in the treatment of a human in need thereof with an ocular disease or disorder, specifically, a disease or disorder recited herein.

In one embodiment, the invention is directed to a compound described herein, or a pharmaceutically acceptable salt thereof, in the manufacture of a medicament for use in the treatment of an ocular disease or disorder, for example the diseases and disorders recited herein. Specifically, the invention further provides for the use of a compound of Formula (I), or a pharmaceutically acceptable salt thereof, in the manufacture of a medicament for use in the treatment of an ocular disease or disorder, for example the diseases and disorders recited herein.

### BRIEF DESCRIPTION OF THE DRAWINGS

FIG. 1 depicts intraocular pressure in Japanese White rabbits after topical administration of 1-(6-(2-(2-methyl-4-(4,4,4-trifluorobutoxy)phenethyl)phenyl)pyridin-2-yl)-5-(trifluoromethyl)-1H-pyrazole-4-carboxylic acid, a reference compound (Comp A) or vehicle to the right eye at time T=0.

FIG. 2 depicts intraocular pressure in Japanese White rabbits after intravitreal administration of 1-(6-(2-(2-methyl-4-(4,4,4-trifluorobutoxy)phenethyl)phenyl)pyridin-2-yl)-5-(trifluoromethyl)-1H-pyrazole-4-carboxylic acid, a reference compound (Comp A) or vehicle to the right eye at time T=0.

FIG. 3. depicts intraocular pressure in C57BL/6J mice after topical administration of 1-(6-(2-(2-methyl-4-(4,4,4-trifluorobutoxy)phenethyl) phenyl)pyridin-2-yl)-5-(trifluoromethyl)-1 H-pyrazole-4-carboxylic acid, latanoprost or vehicle to the right eye at time T=0.

### DETAILED DESCRIPTION OF THE INVENTION

The invention relates to activators of soluble guanylate cyclase (sGC) and their use in pharmaceutical compositions for the reduction of IOP. In particular, the invention relates to a compound of Formula (I):

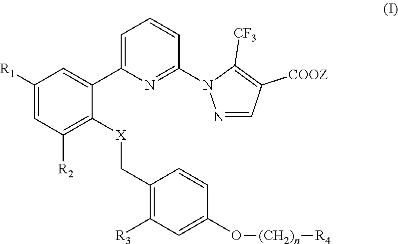

or a pharmaceutically acceptable salt thereof, wherein:
R1 and R2 are each independently selected from —H and halogen (suitably Cl, F, Br, I; preferably Cl, F);
R3 is selected from H, —CH₃ and F;
R4 is selected from —CF₃, —OCH₃, —CN, —COOH, morpholine, 3-(trifluoromethyl)-1-pyrazolyl, an optionally substituted 5- to 6-membered heteroaryl ring, wherein the optional substituents are independently —CN or —OCH₃, and an optionally substituted 5- to 6-membered heterocyclic ring;
X is selected from O and CH₂;
Z is selected from H and C₁₋₄ alkyl; and
n is 2 or 3.

Suitably, R₁ and R₂ are each independently selected from H and halogen. Suitably, the halogen is selected from chlorine, fluorine, bromine and iodine. In one embodiment of the invention, halogen is selected from chlorine and fluorine.

Suitably, X is selected from O and CH₂.

Suitably, R₃ is selected from —H, —CH₃ and fluorine.

Suitably, R₄ is selected from —CF₃, —OCH₃, —CN, —COOH, morpholine, 3-(trifluoromethyl)-1-pyrazolyl, an optionally substituted 5- to 6-membered heteroaryl ring, wherein the optional substituents are —CN, —OCH₃, and an optionally substituted 5- to 6-membered heterocyclic ring.

Suitably, n is an integer from 2 to 3.

In one aspect, the present invention is a compound, or a pharmaceutically acceptable salt thereof, which is described herein:
1-(6-(2-(2-methyl-4-(4,4,4-trifluorobutoxy)phenethyl)phenyl)pyridin-2-yl)-5-(trifluoromethyl)-1H-pyrazole-4-carboxylic acid;
1-(6-(2-(4-(3-morpholinopropoxy)phenethyl)phenyl)pyridin-2-yl)-5-(trifluoromethyl)-1 H-pyrazole-4-carboxylic acid;
1-(6-(2-(2-methyl-4-(3-(3-(trifluoromethyl)-1H-pyrazol-1-yl)propoxy)phenethyl)phenyl)pyridin-2-yl)-5-(trifluoromethyl)-1H-pyrazole-4-carboxylic acid;
1-(6-(2-((4-(2-methoxyethoxy)benzyl)oxy)phenyl)pyridin-2-yl)-5-(trifluoromethyl)-1H-pyrazole-4-carboxylic acid;
1-(6-(2-((2-fluoro-4-(4,4,4-trifluorobutoxy)benzyl)oxy)phenyl)pyridin-2-yl)-5-(trifluoromethyl)-1H-pyrazole-4-carboxylic acid;
1-(6-(2-((2-fluoro-4-(3-methoxypropoxy)benzyl)oxy)phenyl)pyridin-2-yl)-5-(trifluoromethyl)-1H-pyrazole-4-carboxylic acid;
1-(6-(2-((4-(3-methoxypropoxy)benzyl)oxy)phenyl)pyridin-2-yl)-5-(trifluoromethyl)-1H-pyrazole-4-carboxylic acid;
1-(6-(2-((4-(3-cyanopropoxy)benzyl)oxy)phenyl)pyridin-2-yl)-5-(trifluoromethyl)-1H-pyrazole-4-carboxylic acid;
1-(6-(2-((4-(3-cyanopropoxy)-2-methylbenzyl)oxy)phenyl)pyridin-2-yl)-5-(trifluoromethyl)-1H-pyrazole-4-carboxylic acid;
1-(6-(2-((4-(3-methoxypropoxy)-2-methylbenzyl)oxy)phenyl)pyridin-2-yl)-5-(trifluoromethyl)-1H-pyrazole-4-carboxylic acid;
1-(6-(2-((2-methyl-4-(4,4,4-trifluorobutoxy)benzyl)oxy)phenyl)pyridin-2-yl)-5-(trifluoromethyl)-1H-pyrazole-4-carboxylic acid;
1-(6-(2-((4-(3-carboxypropoxy)-2-methylbenzyl)oxy)phenyl)pyridin-2-yl)-5-(trifluoromethyl)-1H-pyrazole-4-carboxylic acid;
1-(6-(5-fluoro-2-(2-methyl-4-(4,4,4-trifluorobutoxy)phenethyl)phenyl)pyridin-2-yl)-5-(trifluoromethyl)-1H-pyrazole-4-carboxylic acid;
1-(6-(2-(4-(3-(1H-pyrazol-1-yl)propoxy)-2-methylphenethyl)-5-fluorophenyl)pyridin-2-yl)-5-(trifluoromethyl)-1H-pyrazole-4-carboxylic acid;
1-(6-(2-(4-(3-(4-cyano-1H-pyrazol-1-yl)propoxy)-2-methylphenethyl)-5-fluorophenyl)pyridin-2-yl)-5-(trifluoromethyl)-1H-pyrazole-4-carboxylic acid;
1-(6-(5-fluoro-2-(4-(3-(4-methoxy-1H-pyrazol-1-yl)propoxy)-2-methylphenethyl)phenyl)pyridin-2-yl)-5-(trifluoromethyl)-1H-pyrazole-4-carboxylic acid;
1-(6-(2-(4-(3-(1H-1,2,4-triazol-1-yl)propoxy)-2-methylphenethyl)-5-fluorophenyl)pyridin-2-yl)-5-(trifluoromethyl)-1H-pyrazole-4-carboxylic acid;
1-(6-(2-(4-(3-(3-cyano-1H-pyrazol-1-yl)propoxy)-2-methylphenethyl)-5-fluorophenyl)pyridin-2-yl)-5-(trifluoromethyl)-1H-pyrazole-4-carboxylic acid;
Ethyl 1-(6-(2-(2-methyl-4-(4,4,4-trifluorobutoxy)phenethyl)phenyl)pyridin-2-yl)-5-(trifluoromethyl)-1H-pyrazole-4-carboxylate;
Isopropyl 1-(6-(2-(2-methyl-4-(4,4,4-trifluorobutoxy)phenethyl)phenyl)pyridin-2-yl)-5-(trifluoromethyl)-1H-pyrazole-4-carboxylate; and
1-(6-(3-chloro-2-(2-methyl-4-(4,4,4-trifluorobutoxy)phenethyl)phenyl)pyridin-2-yl)-5-(trifluoromethyl)-1H-pyrazole-4-carboxylic acid.

A particularly preferred compound of the invention is 1-(6-(2-(2-methyl-4-(4,4,4-trifluorobutoxy)phenethyl)phenyl)pyridin-2-yl)-5-(trifluoromethyl)-1H-pyrazole-4-carboxylic acid.

Another particularly preferred compound of the invention is 1-(6-(2-(2-methyl-4-(3-(3-(trifluoromethyl)-1H-pyrazol-1-yl)propoxy)phenethyl)phenyl)pyridin-2-yl)-5-(trifluoromethyl)-1 H-pyrazole-4-carboxylic acid.

The alternative definitions for the various groups and substituent groups of Formula (I) provided throughout the specification are intended to particularly describe each compound species disclosed herein, individually, as well as groups of one or more compound species. The scope of this invention includes any combination of these group and substituent group definitions. The compounds of the invention are only those which are contemplated to be “chemically stable” as will be appreciated by those skilled in the art.

As used herein, the terms “a compound” or “the compound” refer to one or more compounds of the present invention, particularly, compounds of Formula (I), as defined herein, in any form, i.e., any salt or non-salt form (e.g., as a free acid or base form, or as a salt, particularly a pharmaceutically acceptable salt thereof) and any physical form thereof (e.g., including non-solid forms (e.g., liquid or semi-solid forms), and solid forms (e.g., amorphous or crystalline forms, specific polymorphic forms, solvate forms, including hydrate forms (e.g., mono-, di- and hemi-hydrates)), and mixtures of various forms. The skilled artisan will appreciate that pharmaceutically acceptable solvates may be formed for crystalline compounds wherein solvent molecules are incorporated into the crystalline lattice during crystallization. Solvates may involve non-aqueous solvents such as ethanol, isopropanol, DMSO, acetic acid, ethanolamine, and ethyl acetate, or they may involve water as the solvent that is incorporated into the crystalline lattice. Solvates wherein water is the solvent incorporated into the crystalline lattice are typically referred to as “hydrates.” Hydrates include stoichiometric hydrates as well as compositions containing variable amounts of water. The present invention includes all such solvates and forms.

The present invention includes compounds as well as their pharmaceutically acceptable salts. Accordingly, the word “or” in the context of “a compound or a pharmaceutically acceptable salt thereof” is understood to refer to either a compound or a pharmaceutically acceptable salt thereof (alternative), or a compound and a pharmaceutically acceptable salt thereof (in combination). The following examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

As used herein, the term “pharmaceutically acceptable” refers to those compounds, materials, compositions, and dosage forms which are, within the scope of sound medical judgment, suitable for use in contact with the tissues of human beings and animals without excessive toxicity, irritation, allergic response, or other problem or complication, commensurate with a reasonable benefit/risk ratio. The skilled artisan will appreciate that pharmaceutically acceptable salts of compounds according to formula (I) may be prepared. These pharmaceutically acceptable salts may be prepared in situ during the final isolation and purification of the compound, or by separately reacting with the purified compound in its free acid or free base form with a suitable base or acid, respectively.

Compounds of the present invention can form pharmaceutically acceptable salts by reaction with a suitable base. Suitable bases include, for example, hydroxides, carbonates, hydrides, and alkoxides including NaOH, KOH, Na₂CO₃, K₂CO₃, NaH, potassium-t-butoxide, ammonium salts, and Trometamol which is a tris-salt as trishydroxymethyllaminomethane or 2-amino-2-hydroxymethyl-1,3-propanediol.

In a further aspect, the invention provides a method of treating a disease comprising administering the compound of the present invention or a pharmaceutically acceptable salt thereof to a patient in need thereof, wherein the disease is a result of increased IOP, for example glaucoma or ocular hypertension.

“Alkyl” refers to a saturated, straight or branched hydrocarbon group having the specified number of carbon atoms. The term “(C₁-C₄)alkyl” refers to an alkyl moiety containing from 1 to 4 carbon atoms. Exemplary alkyls include, but are not limited to methyl, ethyl, n-propyl, isopropyl, n-butyl, isobutyl, s-butyl, and t-butyl.

“Alkenyl” refers to straight or branched hydrocarbon group having at least 1 and up to 3 carbon-carbon double bonds. Examples include ethenyl and propenyl.

“Alkoxy” refers to an “alkyl-oxy-” group, containing an alkyl moiety attached through an oxygen linking atom. For example, the term “(C₁-C₄)alkoxy” represents a saturated, straight or branched hydrocarbon moiety having at least 1 and up to 4 carbon atoms attached through an oxygen linking atom. Exemplary “(C₁-C₄)alkoxy” groups include, but are not limited to, methoxy, ethoxy, n-propoxy, isopropoxy, n-butoxy, s-butoxy, and t-butoxy.

A carbocyclic group is a cyclic group in which all of the ring members are carbon atoms, which may be saturated, partially unsaturated (non-aromatic) or fully unsaturated (aromatic). The term “carbocyclic” includes cycloalkyl and aryl groups.

“Cycloalkyl” refers to a non-aromatic, saturated, cyclic hydrocarbon group containing the specified number of carbon atoms. For example, the term “(C₃-C₆)cycloalkyl” refers to a non-aromatic cyclic hydrocarbon ring having from three to six ring carbon atoms. Exemplary “(C₃-C₆)cycloalkyl” groups include cyclopropyl, cyclobutyl, cyclopentyl, and cyclohexyl.

“Aryl” refers to a group or moiety comprising an aromatic, monocyclic or bicyclic hydrocarbon radical containing from 5- to 10-carbon ring atoms and having at least one aromatic ring. Examples of “aryl” groups are phenyl, naphthyl, indenyl, and dihydroindenyl (indanyl). Generally, in the compounds of this invention, aryl is phenyl.

A heterocyclic group is a 5- to 6-membered cyclic group having, as ring members, atoms of at least two different elements, which cyclic group may be saturated, partially unsaturated (non-aromatic) or fully unsaturated (aromatic). The terms “heterocyclic” or “heterocyclyl” includes heterocycloalkyl and heteroaryl groups. Examples of “heterocyclic” groups include, but are not limited to, oxadiazolone.

“Heterocycloalkyl” refers to a saturated, non-aromatic, monocyclic or bicyclic group containing 3-10 ring atoms containing one or more (generally one or two) heteroatom substitutions independently selected from oxygen, sulfur, and nitrogen. Examples of “heterocycloalkyl” groups include, but are not limited to, aziridinyl, thiiranyl, oxiranyl, azetidinyl, oxetanyl, thietanyl, pyrrolidinyl, tetrahydrofuranyl, tetrahydrothienyl, piperidinyl, piperazinyl, tetrahydropyranyl, tetrahydrothiopyranyl, 1,4-dioxanyl, 1,4-oxathiolanyl, 1,4-oxathianyl, 1,4-dithianyl, morpholinyl, thiomorpholinyl, hexahydro-1H-1,4-diazepinyl, azabicylo[3.2.1]octyl, azabicylo[3.3.1]nonyl, azabicylo[4.3.0]nonyl, oxabicylo[2.2.1]heptyl, and 1,1-dioxidotetrahydro-2H-thiopyranyl.

The term “5-6-membered heterocycloalkyl” refers to a non-aromatic, monocyclic group, which is saturated, containing 5 or 6 ring atoms, which includes one or two heteroatoms selected independently from oxygen, sulfur, and nitrogen. Illustrative examples of 5 to 6-membered heterocycloalkyl groups include, but are not limited to pyrrolidinyl, piperidinyl, piperazinyl, tetrahydrofuranyl, tetrahydrothienyl, tetrahydropyranyl, tetrahydrothiopyranyl, morpholinyl, and thiomorpholinyl.

“Heteroaryl” refers to a group or moiety comprising an aromatic monocyclic or bicyclic radical, containing 5- to 10-ring atoms, including 1 to 4 heteroatoms independently selected from nitrogen, oxygen and sulfur. This term also encompasses bicyclic heterocyclic-aryl groups containing either an aryl ring moiety fused to a heterocycloalkyl ring moiety or a heteroaryl ring moiety fused to a cycloalkyl ring moiety.

The term “5-6-membered heteroaryl” refers to an aromatic monocyclic group containing 5 or 6 ring atoms, including at least one carbon atom and 1 to 4 heteroatoms independently selected from nitrogen, oxygen and sulfur. Selected 5-membered heteroaryl groups contain one nitrogen, oxygen, or sulfur ring heteroatom, and optionally contain 1, 2, or 3 additional nitrogen ring atoms. Selected 6-membered heteroaryl groups contain 1, 2, or 3 nitrogen ring heteroatoms. Examples of 5-membered heteroaryl groups include furyl (furanyl), thienyl, pyrrolyl, imidazolyl, pyrazolyl, triazolyl, tetrazolyl, thiazolyl, isothiazolyl, thiadiazolyl, oxazolyl, isoxazolyl, oxadiazolyl and oxo-oxadiazolyl. Examples of 6-membered heteroaryl groups include pyridinyl, oxo-pyridyl, pyrazinyl, pyrimidinyl, pyridazinyl and triazinyl.

The terms “halogen” and “halo” refer to chloro, fluoro, bromo, or iodo substituents. “Oxo” represents a double-bonded oxygen moiety; for example, if attached directly to a carbon atom forms a carbonyl moiety (—C═O). “Hydroxy” or “hydroxyl” is intended to mean the radical —OH. As used herein, the term “cyano” refers to the group —CN.

As used herein, the term “optionally substituted” indicates that a group (such as an alkyl, cycloalkyl, alkoxy, heterocycloalkyl, aryl, or heteroaryl group) or ring or moiety (such as a carbocyclic or heterocyclic ring or moiety) may be unsubstituted, or the group, ring or moiety may be substituted with one or more substituent(s) as defined. In the case where groups may be selected from a number of alternative groups, the selected groups may be the same or different.

The term “independently” means that where more than one substituent is selected from a number of possible substituents, those substituents may be the same or different.

A therapeutically “effective amount” is intended to mean that amount of a compound that, when administered to a patient in need of such treatment, is sufficient to effect treatment, as defined herein. Thus, e.g., a therapeutically effective amount of a compound of Formula (I), or a pharmaceutically acceptable salt thereof, is a quantity of an inventive agent that, when administered to a human in need thereof, is sufficient to modulate and/or inhibit the activity of sGC such that a disease condition which is mediated by that activity is reduced, alleviated or prevented. The amount of a given compound that will correspond to such an amount will vary depending upon factors such as the particular compound (e.g., the potency (pIC₅₀), efficacy (EC₅₀), and the biological half-life of the particular compound), disease condition and its severity, the identity (e.g., age, size and weight) of the patient in need of treatment, but can nevertheless be routinely determined by one skilled in the art. Likewise, the duration of treatment and the time period of administration (time period between dosages and the timing of the dosages, e.g., before/with/after meals) of the compound will vary according to the identity of the mammal in need of treatment (e.g., weight), the particular compound and its properties (e.g., pharmacokinetic properties), disease or disorder and its severity and the specific composition and method being used, but can nevertheless be determined by one of skill in the art.

“Treating” or “treatment” is intended to mean at least the mitigation of a disease or disorder in a patient. The methods of treatment for mitigation of a disease or disorder include the use of the compounds in this invention in any conventionally acceptable manner, for example for prevention, retardation, prophylaxis, therapy or cure of an sGC-mediated disease or disorder, as described hereinabove.

In describing the invention, chemical elements are identified in accordance with the Periodic Table of the Elements. Abbreviations and symbols utilized herein are in accordance with the common usage of such abbreviations and symbols by those skilled in the chemical and biological arts. Specifically, the following abbreviations may be used in the examples and throughout the specification:

| | | |
|---|---|---|
| g (grams) | mg (milligrams) | rt (retention time) |
| L (liters) | mL or ml (milliliters) | EtOH (ethanol) |
| μL (microliters) | psi (pounds per square inch) | EtOAc (ethyl acetate) |
| M (molar) | mM (millimolar) | |
| mol (moles) | mmol (millimoles) | |
| RT (room temperature) | MeOH (methanol) | |
| i-PrOH (isopropanol) | TEA (triethylamine) | |
| TFA (trifluoroacetic acid) | TFAA (trifluoroacetic anhydride) | |
| THF (tetrahydrofuran) | DMSO (dimethylsulfoxide) | |

The reactions described herein are applicable for producing compounds of the invention having a variety of different substituent groups (e.g., R¹, R², etc.), as defined herein. The skilled artisan will appreciate that if a particular substituent is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (3rd ed.), John Wiley & Sons, NY (1999).

Schemes

The following schemes illustrate how compounds of the present invention can be prepared. The specific solvents and reaction conditions referred to are also illustrative and are not intended to be limiting. Compounds not described are either commercially available or are readily prepared by one skilled in the art using available starting materials.

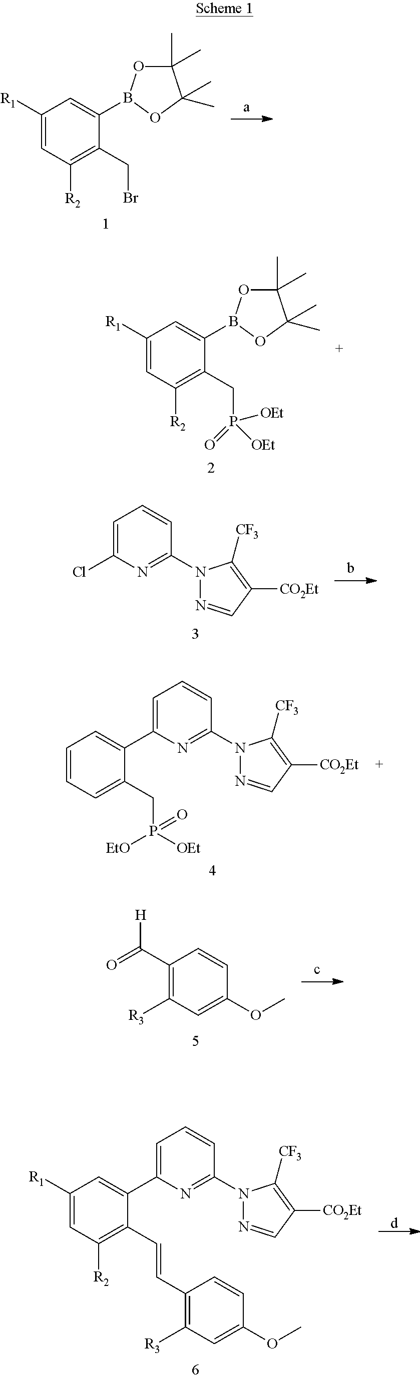

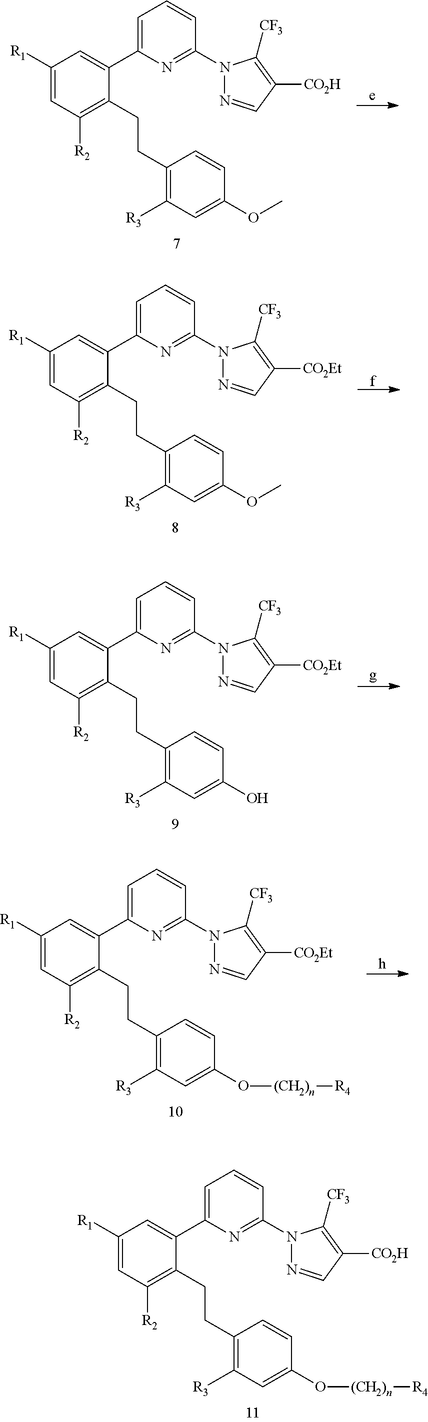

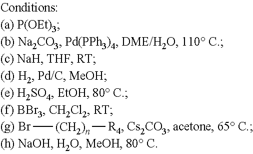

Scheme 1 represents a general scheme for the preparation of compounds according to Formula (I). Boronic acid 1 and aldehyde 5 depicted as starting materials are commercially available or made from commercially available starting materials using methods known to those skilled in the art. Reaction conditions are as described above in the Scheme; however, the skilled artisan will appreciate that certain modifications in the reaction conditions and/or reagents used are possible.

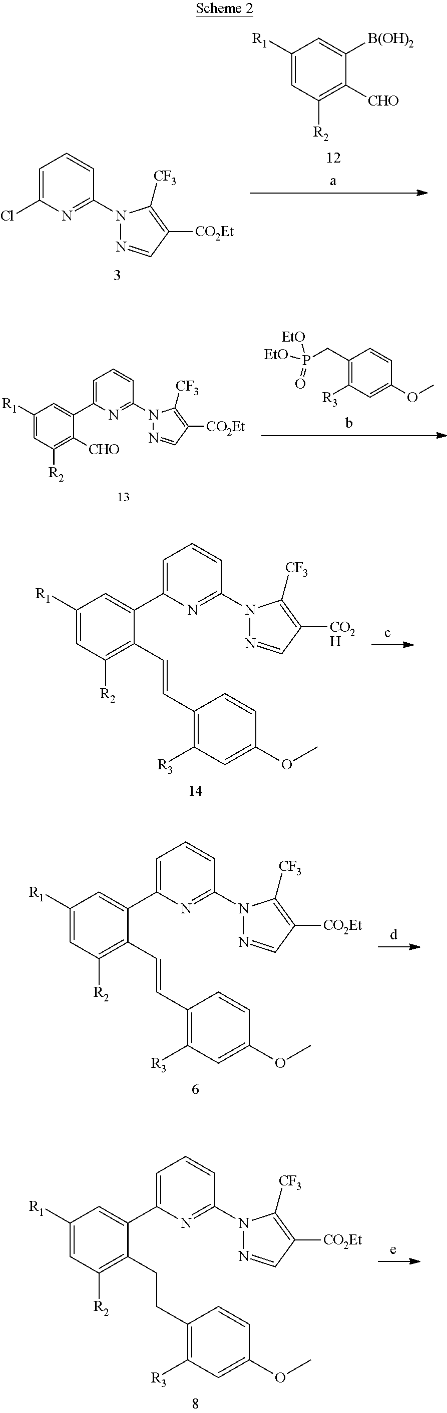

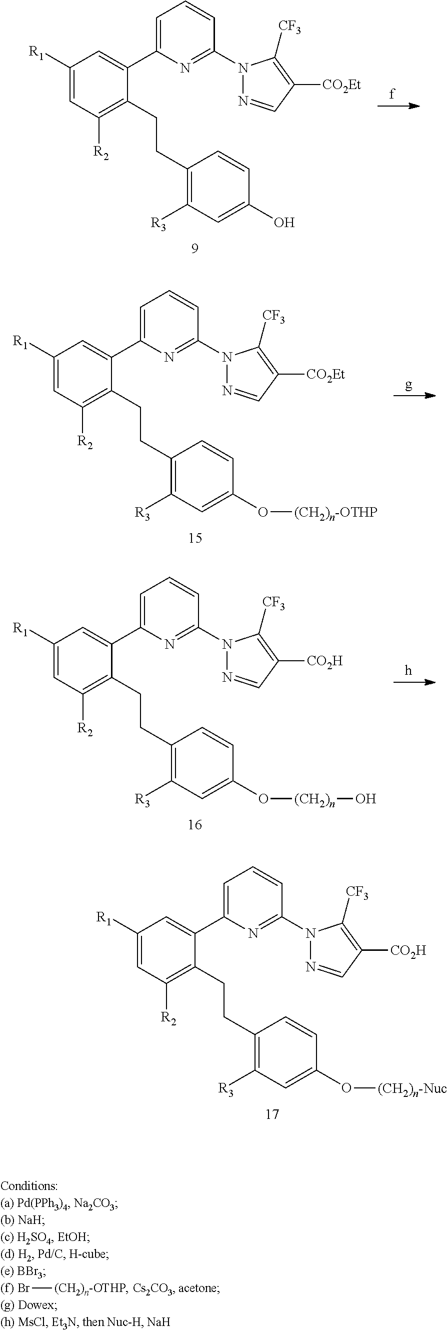

Scheme 2 describes an alternate method for the preparation of compounds of Formula (I) where X is CH₂. The indicated starting materials are commercially available or made from commercially available starting materials using methods known to those skilled in the art. Reaction conditions are as described above in the Scheme; however, the skilled artisan will appreciate that certain modifications in the reaction conditions and/or reagents used are possible. For compounds of Formula (I) where R4 is a heterocycle, an appropriate nucleophile (Nuc) is selected using methods known to those skilled in the art. Some examples of useful nucleophiles are morpholine or 3 or 4-substituted pyrazole or triazole.

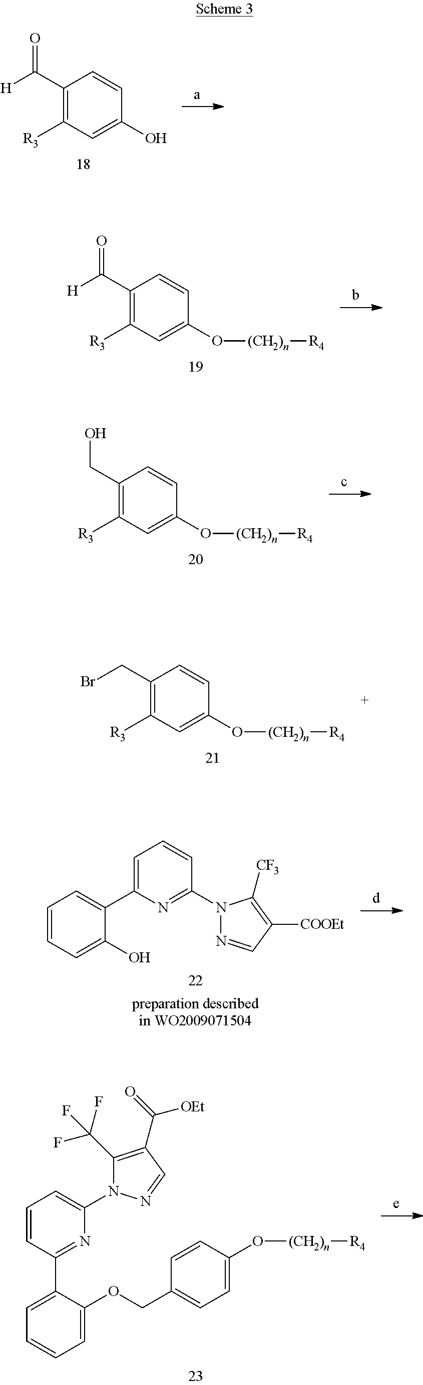

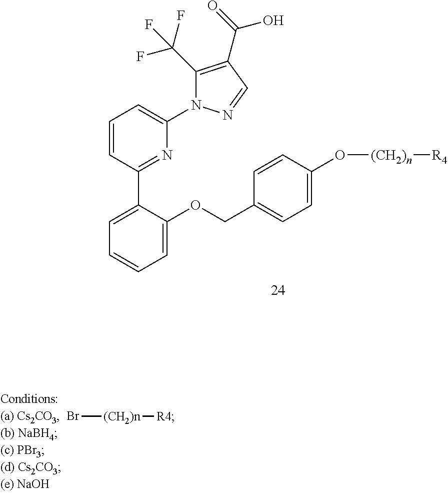

Scheme 3 represents a general scheme for the preparation of compounds according to Formula (I) where X is O. Aldehyde 12 depicted as starting material is commercially available or made from commercially available starting materials using methods known to those skilled in the art. Reaction conditions are as described above in the scheme; however, the skilled artisan will appreciate that certain modifications in the reaction conditions and/or reagents used are possible.

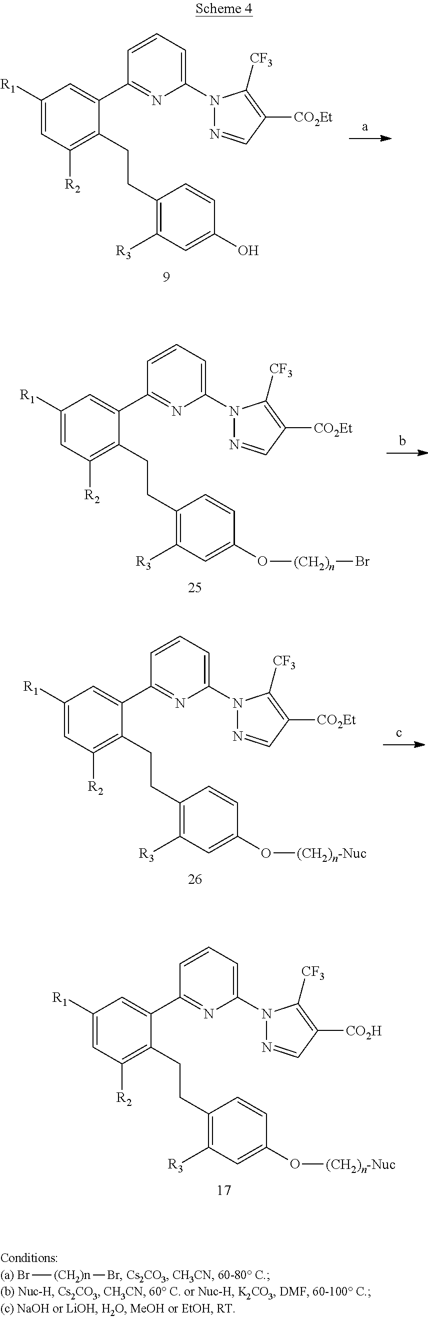

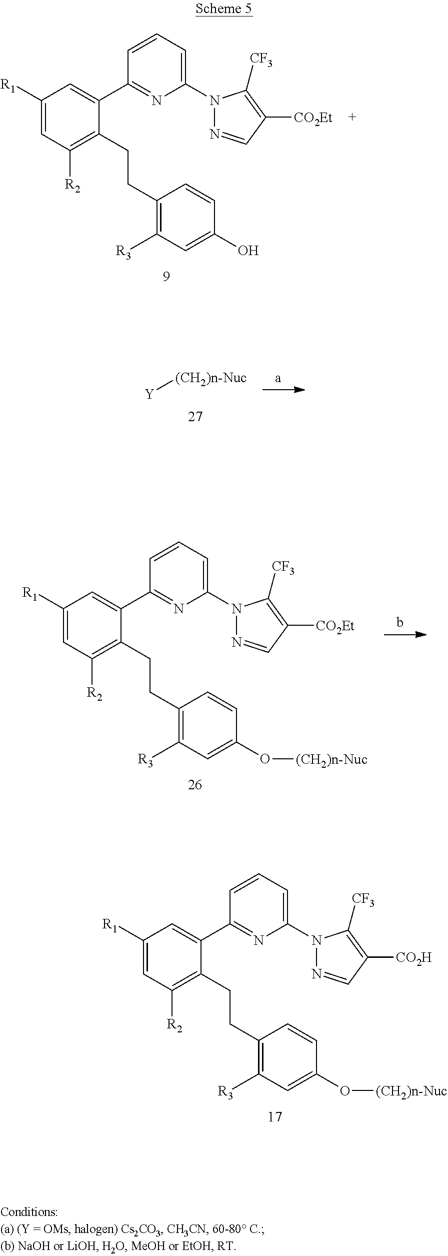

Schemes 4 and 5 describe alternate methods for the preparation of compounds of Formula (I) wherein X is CH₂ starting from intermediate 9 (preparation described in schemes 1 or 2).

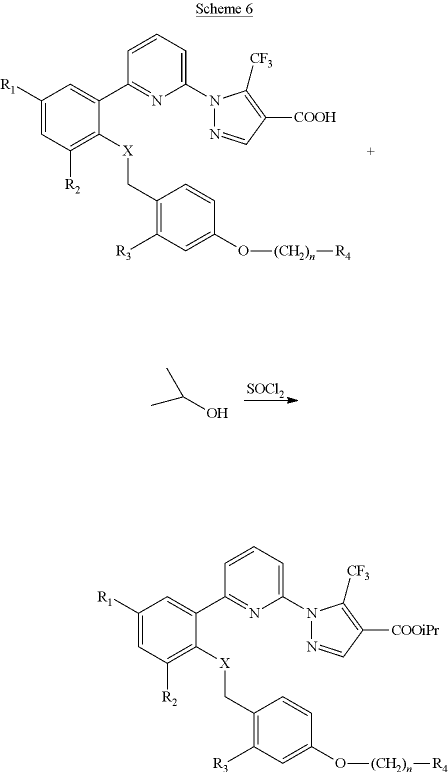

Scheme 6 describes a method for the preparation of compounds of Formula (I) wherein Z is C₁₋₄ alkyl

### EXAMPLES

The following examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

Unless otherwise noted, all starting materials were obtained from commercial suppliers and used without further purification. Unless otherwise indicated, all temperatures are expressed in ° C. (degrees Centigrade). Unless otherwise indicated, all reactions are conducted under an inert atmosphere at room temperature.

The compounds of Examples 1-12 were purified by silica chromatography. Preparative HPLC refers to methods where the material was purified by high pressure liquid chromatography. Unless otherwise stated, silica flash column chromatography refers to the purification of material using Redisep™ pre-packed silica flash columns on an ISCO sq16x machine with the stated solvent systems.

The compounds of Examples 13-23 were purified by silica chromatography. Preparative HPLC refers to methods where the material was purified by high pressure liquid chromatography. Preparative HPLC instruments used were as follows:

Prep-HPLC Instrument: Waters 2545, 2707 Auto sampler with WFC III Fraction collection

Method A: Column: X Terra C18 (250*19 mm) 10μ Mobile Phase, A=0.1% ammonium bicarbonate (63%) and B=acetonitrile (37%); Flow rate, 18 ml/min; Sample loading solvent acetonitrile+MeOH; Fraction volume 200 mL

Method B: Column: XBridge C18 (150*30 mm, 5μ); Mobile Phase, A=0.1% formic acid in water, B=acetonitrile Gradient Time (min)/% B: 0/10, 2/10, 15/60, 18/90; Column Temp ° C.: Ambient; Flow rate, 30 ml/min, Sample loading solvent ACN+THF; Fraction volume, 150 mL

Method C: Column: Sunfire C18 (150*30 mm, 5μ); Mobile Phase, A=0.1% formic acid in water, B=acetonitrile Gradient Time (min)/% B: 0/10, 1/10, 15/60; Column Temp ° C.: Ambient; Flow rate, 30 ml/min, Sample loading solvent ACN+methanol; Fraction volume, 150 mL

Method D: Column: XBridge C18 (150×30 mm) 5μ; Mobile Phase A=10 mm ammonium bicarbonate; B=acetonitrile (40:60); Temp, ambient; Flow rate, 30 ml/min; Sample loading solvent, acetonitrile; Fraction volume, 150 mL

For the compounds of Examples 1-12, analytical HPLC was conducted on a X-terra MS C18 column (2.5 μm 3*30 mm id) eluting with 0.01M ammonium acetate in water (solvent A) and 100% acetonitrile (solvent B) using the following elution gradient: 0→4 minutes, 5% B→100% B; 4→5 minutes, 100% B at a flowrate of 1.1 mL/min with a temperature of 40° C. The mass spectra (MS) were recorded on a Micromass ZQ-LC mass spectrometer using electrospray positive ionisation [ES+ve to give MH⁺ molecular ion] or electrospray negative ionisation [ES-ve to give (M−H)⁻ molecular ion] modes.

For the compounds of Examples 1-12, high resolution MS data were captured using one of the following two methods:

(a) Analytical HPLC was conducted on a LUNA 3u C18 column (2.5 μm 30*3 mm id) eluting with 0.01M ammonium acetate in water (solvent A) and 100% acetonitrile (solvent B) using the following elution gradient: 0→0.5 minutes, 5% B; 0.5→3.5 minutes, 5% B→100% B; 3.5→4 minutes, 100% B; 4→4.5 minutes, 100% B→5% B; 4.5→5.5 minutes, 5% B at a flowrate of 1.3 mL/min with a temperature of 40° C. The mass spectra (MS) were recorded on a Micromass LCT mass spectrometer using electrospray positive ionisation [ES+ve to give MH⁺ molecular ion] or electrospray negative ionisation [ES-ve to give (M−H)⁻ molecular ion] modes.

(b) Analytical HPLC was conducted on a X-Bridge C18 column (2.5 μm 30*3 mm id) eluting with 0.01M ammonium acetate in water (solvent A) and 100% acetonitrile (solvent B) using the following elution gradient: 0→0.5 minutes, 5% B; 0.5→3.5 minutes, 5% B→100% B; 3.5→4 minutes, 100% B; 4→4.5 minutes, 100% B→5% B; 4.5→5.5 minutes, 5% B at a flowrate of 1.3 mL/min with a temperature of 40° C. The mass spectra (MS) were recorded on a Micromass LCT mass spectrometer using electrospray positive ionisation [ES+ve to give MH⁺ molecular ion] or electrospray negative ionisation [ES-ve to give (M−H)⁻ molecular ion] modes.

For the compounds of Examples 13-23, high resolution MS data were captured using one of the following methods using an LCMS-Instrument WATERS Acquity UPLC with 3100 SQD MS:

Method A: Column: Acquity BEH C18 (50 mm×2.1 mm, 1.7 μM); Mobile Phase: A=0.1% Formic Acid in water; B=0.1% formic acid in acetonitrile Gradient Time (min)/% B: 0/3, 0.4/3, 3.2/98, 3.8/98, 4.2/3, 4.5/3; Column Temp: 35° C., Flow Rate: 0.6 ml/min

Method B: Column: Acquity BEH C18 (50×2.1 mm, 1.7 μM); Mobile Phase:=A 0.1% Formic Acid in water; B=0.1% formic acid in acetonitrile Gradient Time (min)/% B: 0/3, 1.5/100, 1.9/100, 2/3. Column Temp: 40° C., Flow Rate: 1.0 ml/min

Method C: Column: XBridge C18 (50×4.6 mm, 2.5 μM); Mobile Phase: C=acetonitrile; D=5 mM ammonium acetate in water; Gradient Time (min)/% C: 0/5, 0.5/5, 1/15, 3.3/98, 5.2/98, 5.5/5, 6/5; Column Temp: 35° C., Flow Rate: 1.3 ml/min

Method D: Column: XBridge C18 (50×4.6 mm, 2.5 μM); Mobile Phase: A=5 mM ammonium bicarbonate in water (pH-10); B=acetonitrile; Gradient Time (min)/% D: 0/5, 0.5/5, 1/15, 3.3/98, 5.2/98, 5.5/5, 6/5; Column Temp: 35° C., Flow Rate: 1.3 ml/min

Method E: Column: XBridge C18 (50×2.1 mm, 2.5 μM); Mobile Phase: C=0.1% formic acid in acetonitrile; D=0.1% formic acid in water; Gradient Time (min)/% C: 0/5, 0.5/5, 1/15, 3.3/98, 5.2/98, 5.5/5, 6/5; Column Temp: 35° C., Flow Rate: 1.3 ml/min

Method F: Column: Acquity BEH C18 (100 mm×2.1 mm, 1.7 μM); Mobile Phase: A=0.1% trifluoroacetic acid in water; B=0.1% trifluoroacetic acid in acetonitrile; Gradient Time (min)/% B: 0/3, 8.5/100, 9.0/100, 9.5/3, 10.01/3; Column Temp: 50° C., Flow Rate: 0.55 ml/min

For the compounds of Examples 1-12, in the reporting of Proton Magnetic Resonance CH NMR 300 MHz, Brucker) spectral data, chemical shifts are reported in ppm (δ) using tetramethylsilane as the internal standard. Splitting patterns are designed as s, singlet; d, doublet; t, triplet; q, quartet; m, multiplet.

For the compounds of Examples 13-23, all NMR experiments were recorded in 400 MHz Varian instrument. Solvents used to record NMR experiments are DMSO-d₆ (Cambridge Isotope Laboratories, CIL) & CDCl₃ (CIL) TMS was used as internal standard. All results were interpreted using VNMRJ 3.2 version.

### Example 1

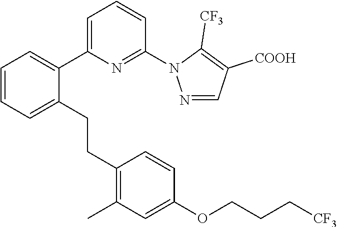 [SMILES: Cc1cc(OCCCC(F)(F)F)ccc1CCc1ccccc1-c1cccc(-n2ncc(C(=O)O)c2C(F)(F)F)n1]

### 1-(6-(2-(2-methyl-4-(4,4,4-trifluorobutoxy)phenethyl)phenyl)pyridin-2-yl)-5-(trifluoromethyl)-1H-pyrazole-4-carboxylic acid

### Intermediate 1: diethyl 2-(4,4,5,5-tetramethyl-1,3,2-dioxaborolan-2-yl)benzylphosphonate

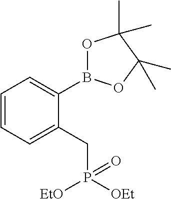 [SMILES: CCOP(=O)(Cc1ccccc1B1OC(C)(C)C(C)(C)O1)OCC]

2-(2-(bromomethyl)phenyl)-4,4,5,5-tetramethyl-1,3,2-dioxaborolane (2 g, 6.73 mmol) and triethyl phosphite (1.23 g, 7.41 mmol) were stirred at 60° C. overnight. The reaction mixture was diluted with dichloromethane and washed with H₂O. The organic layer was dried over anhydrous Na2SO4, filtered and concentrated in vacuo to afford the title compound as a colorless oil (2.35 g, 6.63 mmol, 99%). LC/MS rt=3.25 min (M+H=355 m/z)

### Intermediate 2: ethyl 1-(6-(2-((diethoxyphosphoryl)methyl)phenyl)pyridin-2-yl)-5-(trifluoromethyl)-1H-pyrazole-4-carboxylate

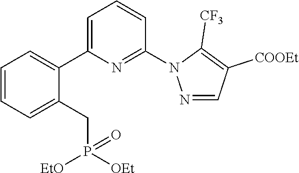 [SMILES: CCOC(=O)c1cnn(-c2cccc(-c3ccccc3CP(=O)(OCC)OCC)n2)c1C(F)(F)F]

Diethyl 2-(4,4,5,5-tetramethyl-1,3,2-dioxaborolan-2-yl)benzylphosphonate (1.463 g, 4.13 mmol), sodium carbonate (0.729 g, 6.88 mmol), Pd(PPh₃)₄ (0.199 g, 0.172 mmol), and ethyl 1-(6-chloropyridin-2-yl)-5-(trifluoromethyl)-1H-pyrazole-4-carboxylate (1.1 g, 3.44 mmol, prepared according to the procedure described in WO 2009/071504) were stirred at reflux overnight. The reaction mixture was concentrated in vacuo, diluted with EtOAc and washed with H₂O. The organic layer was dried over anhydrous Na₂SO₄, filtered and concentrated in vacuo. The product was purified by chromatography on an Isco Companion. The sample was loaded on 12 g AIT silica (Si) column, and purification was carried out using DCM/MeOH 100/0 to 98/2. The appropriate fractions were combined and concentrated in vacuo to give the required product as an off-white oil (1.55 g, 3.03 mmol, 88%). LC/MS rt=3.33 min, (M+H)=512.

### Intermediate 3: (E)-1-(6-(2-(4-methoxy-2-methylstyryl)phenyl)pyridin-2-yl)-5-(trifluoromethyl)-1H-pyrazole-4-carboxylic acid

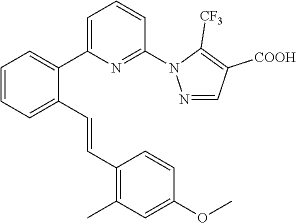 [SMILES: COc1ccc(/C=C/c2ccccc2-c2cccc(-n3ncc(C(=O)O)c3C(F)(F)F)n2)c(C)c1]

To a suspension of ethyl 1-(6-(2-((diethoxyphosphoryl)methyl)phenyl)pyridin-2-yl)-5-(trifluoromethyl)-1H-pyrazole-4-carboxylate (1.55 g, 3.03 mmol) in tetrahydrofuran (THF) (15 ml) at room temperature was added NaH (0.255 g, 6.36 mmol). The resulting suspension was stirred 1 h and 4-methoxy-2-methylbenzaldehyde (0.501 g, 3.33 mmol) was added. The reaction was stirred at room temperature for 2 days.

LC/MS analysis of the reaction showed the reaction was complete and the ester had been hydrolyzed to the acid. The reaction mixture was concentrated in vacuo, quenched with HCl 1N, and extracted with EtOAc. The organic layer was dried over anhydrous Na₂SO₄, filtered and concentrated in vacuo. The product was purified by chromatography on a Isco Companion. The sample was loaded on 40 g AIT silica (Si) column then the purification was carried out using a DCM/MeOH 100/0 to 98/2. The appropriate fractions were combined and concentrated in vacuo to give the required product (1.2 g, 2.5 mmol, 83%) as an off-white amorphous solid. LC/MS rt=2.79 min, (M−H)=478.

### Intermediate 4: 1-(6-(2-(4-methoxy-2-methylphenethyl)phenyl)pyridin-2-yl)-5-(trifluoromethyl)-1H-pyrazole-4-carboxylic acid

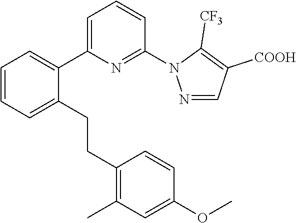 [SMILES: COc1ccc(CCc2ccccc2-c2cccc(-n3ncc(C(=O)O)c3C(F)(F)F)n2)c(C)c1]

The reaction was hydrogenated using the H-cube (settings: 45° C., 1 bar, 1 mL/min) and 10% Pd/C as the catalyst. The reaction mixture was evaporated to give 1.2 g of an off-white oil. LC/MS rt=2.87 min, (M−H)=480.

### Intermediate 5: ethyl 1-(6-(2-(4-methoxy-2-methylphenethyl)phenyl)pyridin-2-yl)-5-(trifluoromethyl)-1H-pyrazole-4-carboxylate

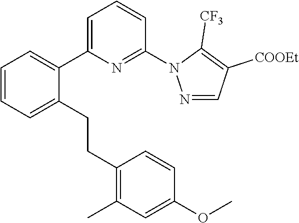 [SMILES: CCOC(=O)c1cnn(-c2cccc(-c3ccccc3CCc3ccc(OC)cc3C)n2)c1C(F)(F)F]

1-(6-(2-(4-methoxy-2-methylphenethyl)phenyl)pyridin-2-yl)-5-(trifluoromethyl)-1H-pyrazole-4-carboxylic acid (1.2 g, 2.5 mmoL) and sulfuric acid (1 mL, 18.76 mmol) were combined in 50 mL ethanol and stirred at 80° C. overnight. Evaluation of the reaction by thin layer chromatography showed the reaction was complete. The reaction mixture was concentrated in vacuo, diluted with dichloromethane, and washed with satd. NaHCO₃ and H₂O. The organic layer was dried over anhydrous Na₂SO₄, filtered and concentrated in vacuo. The product was purified by chromatography on a Isco Companion. The sample was loaded on 50 g Biotage silica (Si) column, and the purification was carried out using 100% dichloromethane. The appropriate fractions were combined and concentrated in vacuo to give the title compound (1 g, 79%) as a yellow oil.

### Intermediate 6: ethyl 1-(6-(2-(4-hydroxy-2-methylphenethyl)phenyl)pyridin-2-yl)-5-(trifluoromethyl)-1H-pyrazole-4-carboxylate

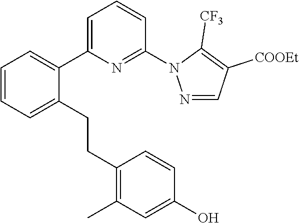 [SMILES: CCOC(=O)c1cnn(-c2cccc(-c3ccccc3CCc3ccc(O)cc3C)n2)c1C(F)(F)F]

To a solution of ethyl 1-(6-(2-(4-methoxy-2-methylphenethyl)phenyl)pyridin-2-yl)-5-(trifluoromethyl)-1H-pyrazole-4-carboxylate (1 g, 1.963 mmol) in Dichloromethane (DCM) (15 ml) at 0° C. was added dropwise BBr₃ (2321 μL, 2.321 mmol). The mixture was stirred overnight 2 days at RT. Analysis by TLC showed the reaction was complete. The reaction mixture was quenched with H₂O and concentrated in vacuo. The residue was dissolved in EtOAc. The organic layer was dried over anhydrous Na₂SO₄, filtered and concentrated in vacuo. The product was purified by chromatography on a Isco Companion. The sample was loaded on 40 g AIT silica (Si) column and eluted with cyclohexane/EtOAc (100:0 to 80:20). The appropriate fractions were combined and concentrated in vacuo to give the required product as a white oil which solidified (930 mg, 81%)

### Intermediate 7: ethyl 1-(6-(2-(2-methyl-4-(4,4,4-trifluorobutoxy)phenethyl)phenyl)pyridin-2-yl)-5-(trifluoromethyl)-1H-pyrazole-4-carboxylate

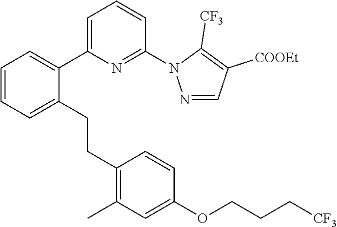 [SMILES: CCOC(=O)c1cnn(-c2cccc(-c3ccccc3CCc3ccc(OCCCC(F)(F)F)cc3C)n2)c1C(F)(F)F]

To a solution of ethyl 1-(6-(2-(4-hydroxy-2-methylphenethyl)phenyl)pyridin-2-yl)-5-(trifluoromethyl)-1H-pyrazole-4-carboxylate (300 mg, 0.605 mmol) in acetone (15 ml) was added Cs₂CO₃ (296 mg, 0.908 mmol). After stirring 30 min 4-bromo-1,1,1-trifluorobutane (0.099 ml, 0.727 mmol) was added and the reaction was heated overnight at 65° C. Analysis by TLC showed the reaction was complete. The reaction mixture was concentrated in vacuo, diluted with dichloromethane and washed with H₂O. The organic layer was dried over anhydrous Na₂SO₄, filtered and concentrated in vacuo. The product was purified by chromatography on a Isco Companion. The sample was loaded on 10 g Biotage silica (Si) column then the purification was carried out using a cyclohexane/EtOAc 100% to 80%. The appropriate fractions were combined and concentrated in vacuo to give the required product as a yellow oil. LC/MS rt=4.59 min, M+H m/z=606.

### Final compound: 1-(6-(2-(2-methyl-4-(4,4,4-trifluorobutoxy)phenethyl)phenyl)pyridin-2-yl)-5-(trifluoromethyl)-1H-pyrazole-4-carboxylic acid

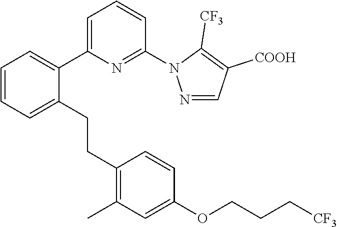 [SMILES: Cc1cc(OCCCC(F)(F)F)ccc1CCc1ccccc1-c1cccc(-n2ncc(C(=O)O)c2C(F)(F)F)n1]

Intermediate 7 ethyl 1-(6-(2-(2-methyl-4-(4,4,4-trifluorobutoxy)phenethyl)phenyl)pyridin-2-yl)-5-(trifluoromethyl)-1H-pyrazole-4-carboxylate (350 mg, 0.578 mmol) and sodium hydroxide (1.16 mL, 1M solution) were combined in MeOH (10 mL) and stirred at 80° C. overnight. Analysis of the reaction by TLC showed the reaction was complete. The reaction was quenched by addition of 1N HCl (2 equiv.). The reaction mixture was concentrated in vacuo, diluted with EtOAc, and washed with H₂O. The organic layer was dried over anhydrous Na₂SO₄, filtered and concentrated in vacuo. The product was purified by chromatography on an Isco Companion. The sample was loaded on 10 g Biotage silica (Si) column then the purification was carried out using a DCM/MeOH 100/0 to 98/2. The appropriate fractions were combined and concentrated in vacuo to give the title compound (260 mg, 68%) as a white oil which solidified. HRMS rt=2.97 min; (M+H) Calculated=578.1878. Found=578.1841.

1H NMR (d6-DMSO) δ (ppm): 8.3 (s, 1H), 8.2 (t, 1H), 7.8 (d, 1H), 7.7 (d, 1H), 7.35 (m, 4H), 6.65 (m, 2H), 6.55 (d, 1H), 4 (m, 2H), 2.8 (m, 2H), 2.6 (m, 2H), 2.4 (m, 2H), 2 (s, 3H), 1.9 (m, 2H).

### Example 2

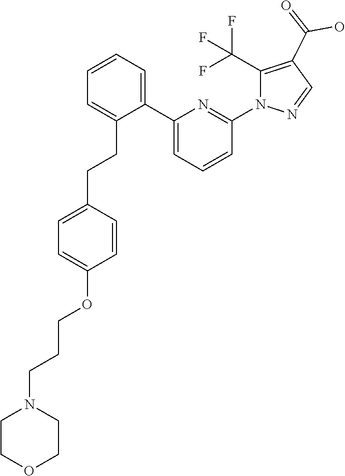 [SMILES: O=C(O)c1cnn(-c2cccc(-c3ccccc3CCc3ccc(OCCCN4CCOCC4)cc3)n2)c1C(F)(F)F]

### 1-(6-(2-(4-(3-morpholinopropoxy)phenethyl)phenyl)pyridin-2-yl)-5-(trifluoromethyl)-1H-pyrazole-4-carboxylic acid

### Intermediate 8: ethyl 1-(6-(2-formylphenyl)pyridin-2-yl)-5-(trifluoromethyl)-1H-pyrazole-4-carboxylate

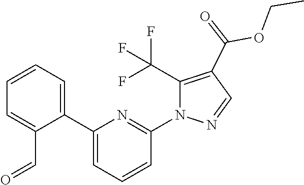 [SMILES: CCOC(=O)c1cnn(-c2cccc(-c3ccccc3C=O)n2)c1C(F)(F)F]

To a solution of ethyl 1-(6-chloro-pyridin-2-yl)-5-(trifluoromethyl)-1H-pyrazole-4-carboxylate (2 g, 6.25 mmol) in DME (50 ml) and H₂O (5 ml), were added Pd(PPh₃)₄ (0.72 g, 0.62 mmol), 2-formylphenylboronic acid (Aldrich, 1.3 g, 8.8 mmol) and Na₂CO₃ (1.3 g, 12.5 mmol. The mixture was heated at 110° C. for 4 hours, then cooled and poured into water. After extraction with AcOEt, the organic phase was dried (Na₂SO₄) and concentrated under reduced pressure. The residue was purified by chromatography on silica gel (CH₂Cl₂). The title compound was obtained as a light brown powder (1.4 g, yield=57.4%) LC/MS: 390.1 (M+H), rt=3.43 min

¹H NMR (CDCl₃, ppm): 10.16 (s, 1H), 8.16 (s, 1H), 8.09 (m, 2H), 7.75 to 7.69 (m, 4H), 7.62 (m, 1H), 4.39 (q, 2H), 1.40 (t, 3H)

### Intermediate 9: (E)-1-(6-(2-(4-methoxystyryl)phenyl)pyridin-2-yl)-5-(trifluoromethyl)-1H-pyrazole-4-carboxylic acid

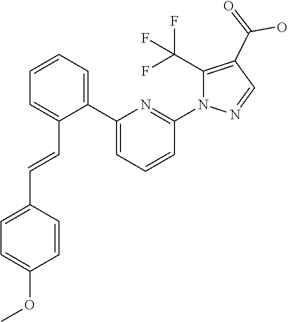 [SMILES: COc1ccc(/C=C/c2ccccc2-c2cccc(-n3ncc(C(=O)O)c3C(F)(F)F)n2)cc1]

To a suspension of diethyl 4-methoxybenzylphosphonate (1.25 g, 2.62 mmol) in Tetrahydrofuran (THF) (15 ml) at room temperature was added NaH (0.126 g, 3.14 mmol). The resulting suspension was stirred 2 h before being cooled in an ice-bath. Ethyl 1-[6-(2-formylphenyl)-2-pyridinyl]-5-(trifluoromethyl)-1H-pyrazole-4-carboxylate (1.121 g, 2.88 mmol) was added and the reaction was stirred overnight at RT. Analysis of the reaction by TLC showed no reaction. An additional portion of NaH (0.126 g, 3.14 mmol) was added at RT, and the mixture was heated at 50° C. for 24 h. Analysis of the reaction by LC/MS showed the reaction was complete, with concomitant hydrolysis of the ester to the acid. The reaction mixture was concentrated in vacuo, diluted with H₂O+2 ml of HCl 1N and extracted with EtOAc. The organic layer was dried over anhydrous Na2SO4, filtered and concentrated in vacuo to give 2 g of 1-[6-(2-{(E)-2-[4-(methyloxy)phenyl]ethenyl}phenyl)-2-pyridinyl]-5-(trifluoromethyl)-1H-pyrazole-4-carboxylic acid (2 g, 4.30 mmol, 148% yield) as a dirty brown oil which was not purified. (LC/MS rt=2.17 min; m/z=466 [M=H]

### Intermediate 10: ethyl 1-[6-(2-{(E)-2-[4-(methyloxy)phenyl]ethenyl}phenyl)-2-pyridinyl]-5-(trifluoromethyl)-1H-pyrazole-4-carboxylate

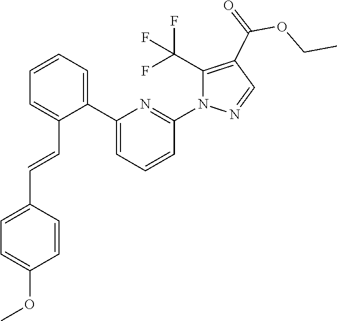 [SMILES: CCOC(=O)c1cnn(-c2cccc(-c3ccccc3/C=C/c3ccc(OC)cc3)n2)c1C(F)(F)F]

1-[6-(2-{(E)-2-[4-(methyloxy)phenyl]ethenyl}phenyl)-2-pyridinyl]-5-(trifluoromethyl)-1H-pyrazole-4-carboxylic acid (2 g, 4.30 mmol, 1 equiv) sulfuric acid (1.0 mL, 18.76 mmol, 1.37 equiv) were stirred at 80° C. overnight. Analysis of the reaction by LC/MS showed the reaction was complete. The reaction mixture was concentrated in vacuo, diluted with DCM and washed with sat NaHCO₃ and H₂O. The organic layer was dried over anhydrous Na2SO4, filtered and concentrated in vacuo. The product was purified by chromatography on a Isco Companion. The sample was loaded on 100 g Biotage silica (Si) column then the purification was carried out using a cyclohexane/AcOEt 100/0 to 80/20. The appropriate fractions were combined and concentrated in vacuo to give the title compound as a orange oil (600 mg, 28%). LC/MS rt=4.27 min m/z 495 [M+H]

### Intermediate 11: ethyl 1-(6-(2-(4-methoxyphenethyl)phenyl)pyridin-2-yl)-5-(trifluoromethyl)-1H-pyrazole-4-carboxylate

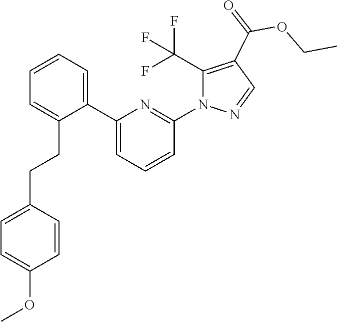 [SMILES: CCOC(=O)c1cnn(-c2cccc(-c3ccccc3CCc3ccc(OC)cc3)n2)c1C(F)(F)F]

Ethyl 1-[6-(2-{(E)-2-[4-(methyloxy)phenyl]ethenyl}phenyl)-2-pyridinyl]-5-(trifluoromethyl)-1H-pyrazole-4-carboxylate (600 mg, 1.216 mmol) was dissolved in methanol (250 mL) and hydrogenated using the H-cube (settings: 40° C., 1 bar, 1 mL/min) and 10% Pd/C as the catalyst. The resulting solution was concentrated in vacuo to give the required product as a yellow oil. (460 mg, 76%). LC/MS rt=4.08 min; m/z=496 [M+H].

### Intermediate 12: ethyl 1-(6-(2-(4-hydroxyphenethyl)phenyl)pyridin-2-yl)-5-(trifluoromethyl)-1 H-pyrazole-4-carboxylate

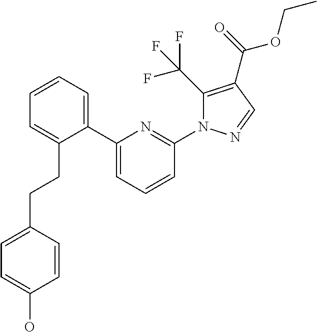 [SMILES: CCOC(=O)c1cnn(-c2cccc(-c3ccccc3CCc3ccc(O)cc3)n2)c1C(F)(F)F]

To a solution of ethyl 1-(6-(2-(4-methoxyphenethyl)phenyl)pyridin-2-yl)-5-(trifluoromethyl)-1H-pyrazole-4-carboxylate (460 mg, 0.928 mmol) in Dichloromethane (DCM) (15 ml) at 0° C. was added dropwise BBr₃ (2321 μL, 2.321 mmol). The mixture was stirred overnight at RT. Analysis of the reaction by LC/MS showed the reaction was complete. The reaction mixture was quenched with H₂O and concentrated in vacuo, then diluted with EtOAc. The organic layer was dried over anhydrous Na₂SO₄, filtered and concentrated in vacuo. The product was purified by chromatography on a Isco Companion. The sample was loaded on 12 g AIT silica (Si) column then the purification was carried out using a Cyclohexane/EtOAc 100/0 to 80/20. The appropriate fractions were combined and concentrated in vacuo to give the required product as a yellow oil (350 mg, 78%). LC/MS rt=3.77 min; m/z 482 [M+H].

### Intermediate 13: ethyl 1-(6-(2-(4-(3-((tetrahydro-2H-pyran-2-yl)oxy)propoxy)phenethyl)phenyl)pyridin-2-yl)-5-(trifluoromethyl)-1H-pyrazole-4-carboxylate

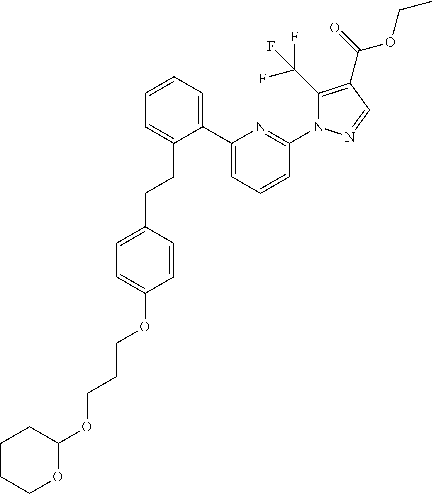 [SMILES: CCOC(=O)c1cnn(-c2cccc(-c3ccccc3CCc3ccc(OCCCOC4CCCCO4)cc3)n2)c1C(F)(F)F]

To a solution of ethyl 1-(6-(2-(4-hydroxyphenethyl)phenyl)pyridin-2-yl)-5-(trifluoromethyl)-1H-pyrazole-4-carboxylate (175 mg, 0.363 mmol) in Acetone (15 ml) was added Cs₂CO₃ (237 mg, 0.727 mmol). After stirring 30 min 2-(3-bromopropoxy)tetrahydro-2H-pyran (0.074 ml, 0.436 mmol) was added and the reaction was heated overnight at 65° C. Analysis of the reaction by TLC showed the reaction was complete. The reaction mixture was concentrated in vacuo, diluted with DCM and washed with H₂O. The organic layer was dried over anhydrous Na₂SO₄, filtered and concentrated in vacuo. The product was purified by chromatography on an Isco Companion. The sample was loaded on 10 g Biotage silica (Si) column then the purification was carried out eluting with cyclohexane/EtOAc 100/0 to 80/20. The appropriate fractions were combined and concentrated in vacuo to give the required product as an off-white oil (120 mg, 52.9%). LC/MS rt=4.51 min m/z=540 [M−THP].

### Intermediate 14: ethyl 1-(6-(2-(4-(3-hydroxypropoxy)phenethyl)phenyl)pyridin-2-yl)-5-(trifluoromethyl)-1H-pyrazole-4-carboxylate

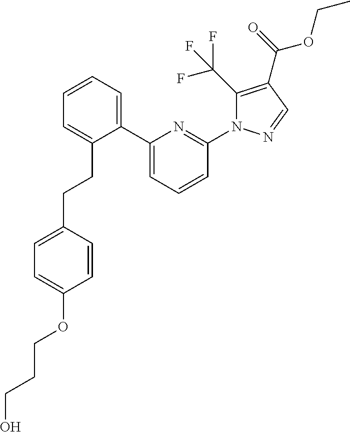 [SMILES: CCOC(=O)c1cnn(-c2cccc(-c3ccccc3CCc3ccc(OCCCO)cc3)n2)c1C(F)(F)F]

Ethyl 1-(6-(2-(4-(3-((tetrahydro-2H-pyran-2-yl)oxy)propoxy)phenethyl)phenyl)pyridin-2-yl)-5-(trifluoromethyl)-1H-pyrazole-4-carboxylate (120 mg, 0.192 mmol) was stirred at RT overnight with a small amount of Dowex H+. Analysis of the reaction by LC/MS showed the reaction was complete. The mixture was filtered and concentrated to give the title compound as an off-white oil (90 mg, 87%). LC/MS rt=3.86 min; m/z=540 [M+H].

### Final compound 1-(6-(2-(4-(3-morpholinopropoxy)phenethyl)phenyl)pyridin-2-yl)-5-(trifluoromethyl)-1H-pyrazole-4-carboxylic acid

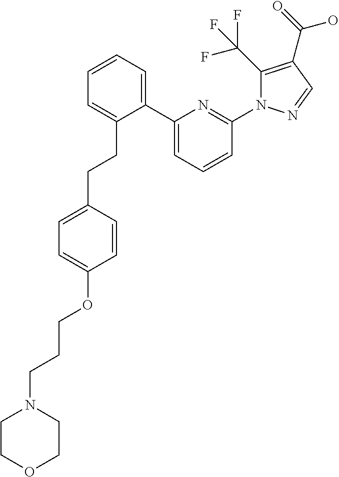 [SMILES: O=C(O)c1cnn(-c2cccc(-c3ccccc3CCc3ccc(OCCCN4CCOCC4)cc3)n2)c1C(F)(F)F]

Intermediate 14 ethyl 1-(6-(2-(4-(3-hydroxypropoxy)phenethyl)phenyl)pyridin-2-yl)-5-(trifluoromethyl)-1H-pyrazole-4-carboxylate (90 mg, 0.167 mmol) and methanesulfonyl chloride (0.044 ml, 0.500 mmol) were combined in dichloromethane (5 mL) and stirred at RT overnight. Analysis of the reaction by LC/MS showed the reaction was incomplete with starting material remaining.

An additional portion of NEt₃ (1 equiv) and methanesulfonyl chloride (1 equiv) were added and the mixture was stirred 4 h at RT. Analysis of the reaction by LC/MS showed the reaction was incomplete with starting material remaining.

An additional portion of NEt₃ (1 equiv) and methanesulfonyl chloride (1 equiv) were added and the mixture was stirred 4 h at RT. Analysis of the reaction by LC/MS showed the reaction was complete. The reaction mixture was washed with H₂O. The organic layer was dried over anhydrous Na₂SO₄, filtered and concentrated in vacuo.

The formation of mesylate compound was observed but the product was not isolated.

The crude mesylate was diluted in THF (10 mL) and morpholine (0.044 ml, 0.500 mmol) was added. The mixture was stirred at RT for 2 days. Analysis of the reaction by LC/MS showed no reaction. NaH (13.34 mg, 0.334 mmol) was added and stirred at 70° C. 2 h. Analysis of the reaction by LC/MS showed the displacement reaction was complete with concomitant hydrolysis of the ester to the acid. The reaction mixture was concentrated in vacuo, diluted with H₂O and quenched with HCl 1N. The resulting mixture was extracted with EtOAc. The organic layer was dried over anhydrous Na₂SO₄, filtered and concentrated in vacuo. The solid residue was taken in iPr₂O to afford the required product as white powder. LC/MS rt=2.82 min m/z=581 [M+H]; HRMS rt=2.59 min, (M+H) Calculated=581.2375. found=581.5404 (Δ=5 ppm).

1H NMR (d6-DMSO) δ (ppm): 8.35 (s, 1H), 8.25 (m, 1H), 7.8 (m, 2H) 7.35 (m, 4H), 6.75 (m, 4H), 4 (t, 2H), 3.75 (m, 4H), 3.1 (m, 2H), 2.85 (m, 4H), 2.65 (m, 2H), 2.5 (m, 2H), 1.95 (m, 2H)

### Example 3

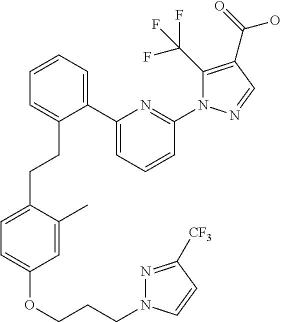 [SMILES: Cc1cc(OCCCn2ccc(C(F)(F)F)n2)ccc1CCc1ccccc1-c1cccc(-n2ncc(C(=O)O)c2C(F)(F)F)n1]

### 1-(6-(2-(2-methyl-4-(3-(3-(trifluoromethyl)-1H-pyrazol-1-yl)propoxy)phenethyl)phenyl)pyridin-2-yl)-5-(trifluoromethyl)-1H-pyrazole-4-carboxylic acid

### Intermediate 15: ethyl 1-(6-(2-(2-methyl-4-(3-((tetrahydro-2H-pyran-2-yl)oxy)propoxy)phenethyl)phenyl)pyridin-2-yl)-5-(trifluoromethyl)-1H-pyrazole-4-carboxylate

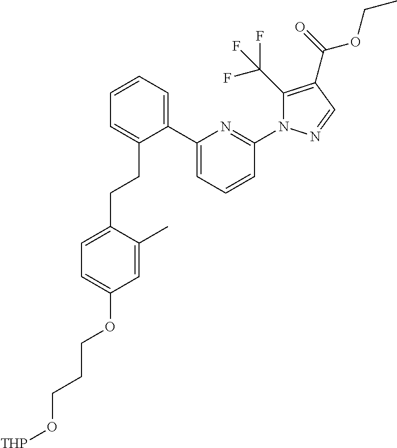

To a solution of ethyl 1-(6-(2-(4-hydroxy-2-methylphenethyl)phenyl)pyridin-2-yl)-5-(trifluoromethyl)-1H-pyrazole-4-carboxylate (600 mg, 1.211 mmol, preparation described in Example 1) in acetone (15 ml) was added Cs₂CO₃ (592 mg, 1.816 mmol). After stirring for 30 min, 2-(3-bromopropoxy)tetrahydro-2H-pyran (324 mg, 1.453 mmol) was added and the reaction was heated overnight at 65° C. Analysis of the reaction by TLC showed the reaction was complete. The reaction mixture was concentrated in vacuo, diluted with DCM and washed with H₂O. The organic layer was dried over anhydrous Na₂SO₄, filtered and concentrated in vacuo. The product was purified by chromatography on a Isco Companion. The sample was loaded on 10 g Biotage silica (Si) column then the purification was carried out using a Cyclohexane/EtOAc 100/0 to 80/20. The appropriate fractions were combined and concentrated in vacuo to give the required product as a colorless oil (710 mg, 92%). LC/MS rt=4.62 min m/z=554 [M+H]−THP.

### Intermediate 16: ethyl 1-(6-(2-(4-(3-hydroxypropoxy)-2-methylphenethyl)phenyl)pyridin-2-yl)-5-(trifluoromethyl)-1H-pyrazole-4-carboxylate

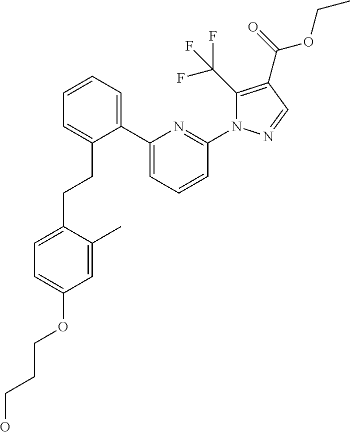 [SMILES: CCOC(=O)c1cnn(-c2cccc(-c3ccccc3CCc3ccc(OCCCO)cc3C)n2)c1C(F)(F)F]

ethyl 1-(6-(2-(2-methyl-4-(3-((tetrahydro-2H-pyran-2-yl)oxy)propoxy)phenethyl)phenyl)pyridin-2-yl)-5-(trifluoromethyl)-1H-pyrazole-4-carboxylate (710 mg, 1.13 mmol) was dissolved in methanol (10 mL) and stirred at RT overnight with a small amount of Dowex H+. Analysis of the reaction by LC/MS showed the reaction was complete. The mixture was filtered and concentrated to give the title compound as a light brown oil (550 mg, 89%). LC/MS rt=3.71 min m/z=554 [M+H].

### Intermediate 17: ethyl 1-(6-(2-(2-methyl-4-(3-((methylsulfonyl)oxy)propoxy)phenethyl)phenyl)-pyridin-2-yl)-5-(trifluoromethyl)-1H-pyrazole-4-carboxylate

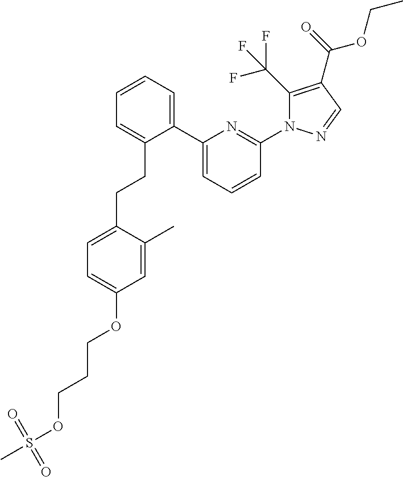 [SMILES: CCOC(=O)c1cnn(-c2cccc(-c3ccccc3CCc3ccc(OCCCOS(C)(=O)=O)cc3C)n2)c1C(F)(F)F]

ethyl 1-(6-(2-(4-(3-hydroxypropoxy)-2-methylphenethyl)phenyl)pyridin-2-yl)-5-(trifluoromethyl)-1H-pyrazole-4-carboxylate (550 mg, 0.994 mmol), methanesulfonyl chloride (285 mg, 2.98 mmol, 0.192 mL) and triethylamine (302 mg, 2.98 mmol, 0.415 mL) were combined in dichloromethane (10 mL) and stirred at RT overnight. Analysis of the reaction by LC/MS showed the reaction was complete. The reaction mixture was washed with H₂O. The organic layer was dried over anhydrous Na₂SO₄, filtered and concentrated in vacuo. The product was purified by chromatography on a Isco Companion. The sample was loaded on 10 g Biotage silica (Si) column then the purification was carried out using cyclohexane/EtOAc 100/0 to 70/30. The appropriate fractions were combined and concentrated in vacuo to give the title compound as an off-white oil (500 mg, 80%). LC/MS rt=4.06 min m/z 632 [M+H].

### Final compound 1-(6-(2-(2-methyl-4-(3-(3-(trifluoromethyl)-1H-pyrazol-1-yl)propoxy)phenethyl)phenyl)pyridin-2-yl)-5-(trifluoromethyl)-1H-pyrazole-4-carboxylic acid

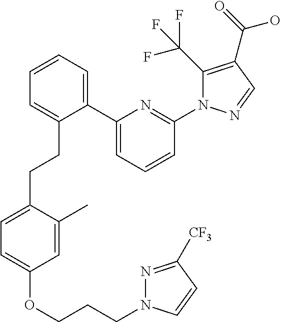 [SMILES: Cc1cc(OCCCn2ccc(C(F)(F)F)n2)ccc1CCc1ccccc1-c1cccc(-n2ncc(C(=O)O)c2C(F)(F)F)n1]

To a solution of Intermediate 17 ethyl 1-(6-(2-(2-methyl-4-(3-((methylsulfonyl)oxy)propoxy)phenethyl)phenyl)-pyridin-2-yl)-5-(trifluoromethyl)-1H-pyrazole-4-carboxylate (232 mg, 0.367 mmol) in tetrahydrofuran (THF) (10 ml) at RT was added NaH (30.9 mg, 0.772 mmol). The suspension was stirred 30 min then 3-(trifluoromethyl)-1H-pyrazole (50 mg, 0.367 mmol) was added. Analysis of the reaction by TLC showed the reaction was complete. 2 equivalents of HCl 1N were added. The reaction mixture was concentrated in vacuo, dissolved in EtOAc and washed with H₂O. The organic layer was dried over anhydrous Na₂SO₄, filtered and concentrated in vacuo. The product was purified by chromatography on a Isco Companion. The sample was loaded on 10 g Biotage silica (Si) column then the purification was carried out using DCM/MeOH 100/0 to 98/2. The appropriate fractions were combined and concentrated in vacuo to give the required product as an off-white oil (180 mg, 76%). LC/MS rt=3.25 min m/z=644 [M+H]. HRMS rt=2.95 min, (M+H) Calculated=644.2096. found=644.2130 (Δ=6.5 ppm).

¹H NMR (d⁶-DMSO) δ (ppm): 8.3 (s, 1H), 8.2 (t, 1H), 8.0 (m, 1H), 7.8 (d, 1H), 7.7 (d, 1H), 7.4 (m, 4H), 6.7 (s, 1H), 6.6 (m, 2H), 6.5 (d, 1H), 4.4 (m, 2H), 3.9 (m, 2H), 2.8 (m, 2H), 2.6 (m, 2H), 2.25 (m, 2H), 1.9 (s, 3H).

### Example 4

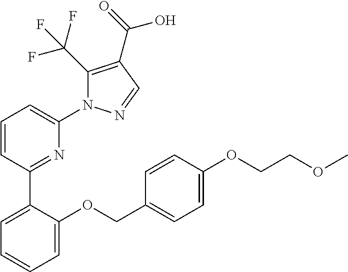 [SMILES: COCCOc1ccc(COc2ccccc2-c2cccc(-n3ncc(C(=O)O)c3C(F)(F)F)n2)cc1]

### 1-(6-(2-((4-(2-methoxyethoxy)benzyl)oxy)phenyl)pyridin-2-yl)-5-(trifluoromethyl)-1 H-pyrazole-4-carboxylic acid

### Intermediate 18: 4-(2-methoxyethoxy)benzaldehyde

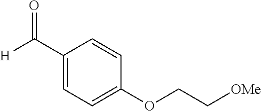 [SMILES: COCCOc1ccc(C=O)cc1]

To a solution of 4-hydroxybenzaldehyde (2.3 g, 18.8 mmol) in acetone was added cesium carbonate (9.2 g, 28.2 mmol, 1.5 eq) and the reaction mixture was stirred at room temperature for 30 minutes. 1-bromo-2-methoxyethane (2.61 g, 18.8 mmol) was added and the reaction mixture was heated under reflux overnight and cooled. After filtration of the insoluble material, the filtrate was concentrated under reduced pressure. The residue was purified by chromatography on silicagel eluting with cyclohexane/ethyl acetate, 9/1 to afford the title compound (1.2 g, 35%) as a yellow oil. ¹H NMR (CDCl₃, ppm): 9.91 (s, 1H), 7.85 (d, 2H), 7.05 (d, 2H), 4.23 (t, 2H), 3.81 (t, 2H), 3.48 (s, 3H).

### Intermediate 19: (4-(2-methoxyethoxy)phenyl)methanol

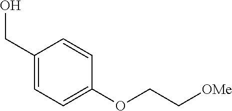 [SMILES: COCCOc1ccc(CO)cc1]

To a solution of 4-(2-methoxyethoxy)benzaldehyde (1.2 g, 6.66 mmol) in EtOH was added portion wise NaBH4 (126 mg, 3.33 mmol, 0.5 eq). The reaction mixture was stirred at room temperature overnight and then poured into water. The mixture was acidified with a solution of 1N HCl. After extraction with ethyl acetate, the organic phase was dried over Na₂SO₄ and concentrated under reduced pressure to afford the title compound (900 mg, 75%) as colorless oil.

### Intermediate 20: 1-(bromomethyl)-4-(2-methoxyethoxy)benzene

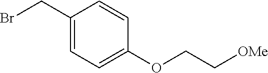 [SMILES: COCCOc1ccc(CBr)cc1]

To a solution of (4-(2-methoxyethoxy)phenyl)methanol (900 mg, 4.94 mmol) in anhydrous CH₂Cl₂ cooled in a ice bath, was added dropwise PBr₃ (1.0 M in CH₂Cl₂, 0.5 eq). The reaction mixture was stirred at 0° C. for 30 minutes, then at room temperature for 2 hours and then was basified with a saturated solution of NaHCO₃. After extraction with CH₂Cl₂, the organic phase was dried over Na₂SO₄ and concentrated under reduced pressure to afford the title compound (1.125 g, 93%) as a yellow oil. ¹H NMR (CDCl₃, ppm): 7.33 (d, 2H), 6.91 (d, 2H), 4.52 (s, 2H), 4.14 (t, 2H), 3.77 (t, 2H), 3.47 (s, 3H).

### Intermediate 21: ethyl 1-(6-(2-((4-(2-methoxyethoxy)benzyl)oxy)phenyl)pyridin-2-yl)-5-(trifluoromethyl)-1H-pyrazole-4-carboxylate

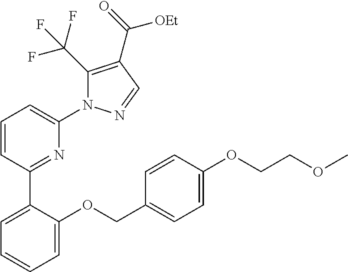 [SMILES: CCOC(=O)c1cnn(-c2cccc(-c3ccccc3OCc3ccc(OCCOC)cc3)n2)c1C(F)(F)F]

To a solution of the ethyl 1-(6-(2-hydroxyphenyl)pyridin-2-yl)-5-(trifluoromethyl)-1H-pyrazole-4-carboxylate (680 mg, 1.8 mmol) in acetone (50 mL) was added cesium carbonate (880 mg, 2.7 mmol, 1.5 eq) and the reaction mixture was stirred at room temperature for 10 minutes. 1-(bromomethyl)-4-(2-methoxyethoxy)benzene (490 mg, 1.28 mmol, 1.1 eq) was added and the reaction mixture was heated at 60° C. for 4 hours and then cooled. The reaction mixture was filtered to remove insoluble material, and the filtrate was concentrated under reduced pressure. The residue was triturated with pentane and the resulting precipitate was collected by filtration and dried to afford the title compound (845 mg, 87%) as a white powder. LC/MS rt=4.04 min, m/z 542.1 [M+H]; ¹H NMR (CDCl₃, ppm): 8.15 (d+s, 2H), 8.00 (dd, 1H), 7.86 (t, 1H), 7.54 (d, 1H), 7.39 (t, 1H), 7.3 (d, 2H), 7.12 (t, 1H), 7.09 (d, 1H), 6.93 (d, 2H), 5.10 (s, 2H), 4.4 (q, 2H), 4.15 (t, 2H), 3.78 (t, 2H), 3.48 (s, 3H), 1.41 (t, 3H).

### Final compound 1-(6-(2-((4-(2-methoxyethoxy)benzyl)oxy)phenyl)pyridin-2-yl)-5-(trifluoromethyl)-1H-pyrazole-4-carboxylic acid

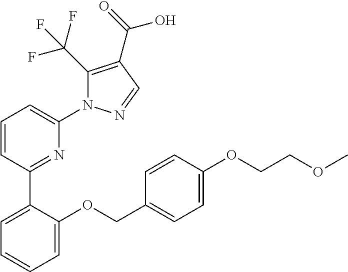 [SMILES: COCCOc1ccc(COc2ccccc2-c2cccc(-n3ncc(C(=O)O)c3C(F)(F)F)n2)cc1]

Intermediate 21 ethyl 1-(6-(2-((4-(2-methoxyethoxy)benzyl)oxy)phenyl)pyridin-2-yl)-5-(trifluoromethyl)-1H-pyrazole-4-carboxylate (840 mg, 1.55 mmol) was dissolved in EtOH and 1N NaOH (2 equiv) was added. The reaction mixture was stirred at room temperature for 2 hours. The solution was concentrated under reduced pressure to remove EtOH and the mixture was acidified to pH-5 with a 1N HCl. After extraction with ethyl acetate, the organic phase was dried over Na₂SO₄ and concentrated under reduced pressure. The residue was triturated with diisopropyl ether, and the resulting precipitate was collected by filtration and dried to afford the title compound (615 mg, 77%) as a cream powder. LC-HRMS: C₂₆H₂₂F₃N₃O₅, rt=2.39 min.

Calc: 512.1434 (M−H) Found: 512.1475 (M−H); ¹H NMR (CDCl₃, ppm): 8.23 (s, 1H), 8.16 (d, 1H), 8.0 (dd, 1H), 7.88 (t, 1H), 7.55 (d, 1H), 7.4 (t, 1H), 7.3 (d, 2H), 7.14 (d, 1H), 7.11 (t, 1H), 6.94 (d, 2H), 5.1 (s, 2H), 4.15 (t, 2H), 3.79 (t, 2H), 3.49 (s, 3H)

The following examples were prepared using procedures analogous to those described in Example 4 using an appropriate aldehyde and alkyl bromide as appropriate and substituting diisopropyl ether for pentane when required for trituration:

| Ex. | Name | LC/HRMS | 1H NMR (ppm) |
|---|---|---|---|
| 5 | 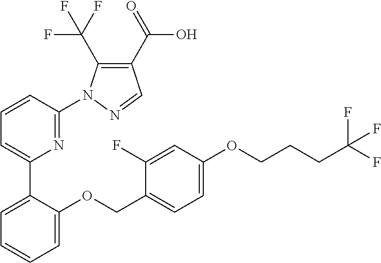 [SMILES: O=C(O)c1cnn(-c2cccc(-c3ccccc3OCc3ccc(OCCCC(F)(F)F)cc3F)n2)c1C(F)(F)F] 1-(6-(2-((2-fluoro-4-(4,4,4- trifluorobutoxy)benzyl)oxy)phenyl)pyridin- 2-yl)-5-(trifluoromethyl)-1H-pyrazole- 4-carboxylic acid | C₂₈H₂₃F₆N₃O₄ rt = 3.00 min Calc: 578.1515 (M − H) Found: 578.1458 (M − H) | CDCl₃: 8.24 (s, 1H), 8.11 (d, 1H), 7.99 (dd, 1H), 7.85 (t, 1H), 7.54 (d, 1H), 7.43 (t, 1H), 7.25 (d, 1H), 7.15 (m, 2H), 6.73 (m, 2H), 5.08 (s, 2H), 4.04 (t, 2H), 2.35 (m, 2H), 2.28 (s, 3H), 2.08 (m, 2H) |
| 6 | 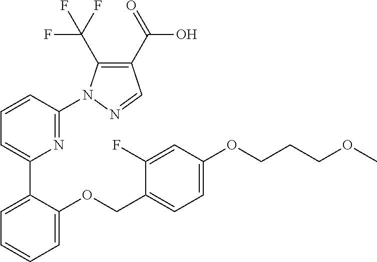 [SMILES: COCCCOc1ccc(COc2ccccc2-c2cccc(-n3ncc(C(=O)O)c3C(F)(F)F)n2)c(F)c1] 1-(6-(2-((2-fluoro-4-(3- methoxypropoxy)benzyl)oxy)phenyl)pyridin- 2-yl)-5-(trifluoromethyl)-1H- pyrazole-4-carboxylic acid | C₂₇H₂₃F₄N₃O₅, rt = 2.72 min Calc: 544.1496 (M − H) Found: 544.1542 (M − H) | d6-DMSO: 8.3 (s, 1H), 8.07 (m, 2H), 7.75 (dd, 1H), 7.68 (dd, 1H), 7.45 (m, 2H), 7.34 (d, 1H), 7.12 (t, 1H), 6.86 (dd, 1H), 6.77 (dd, 1H), 5.18 (s, 2H), 4.03 (t, 2H), 3.45 (t, 2H), 3.24 (s, 3H), 1.94 (m, 2H) |
| 7 | 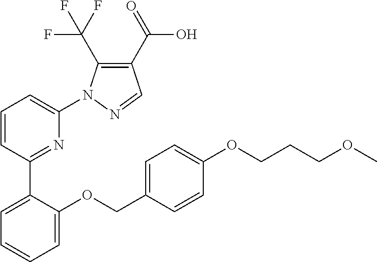 [SMILES: COCCCOc1ccc(COc2ccccc2-c2cccc(-n3ncc(C(=O)O)c3C(F)(F)F)n2)cc1] 1-(6-(2-((4-(3- methoxypropoxy)benzyl)oxy)phenyl)pyridin- 2-yl)-5-(trifluoromethyl)-1H- pyrazole-4-carboxylic acid | C₂₇H₂₄F₃N₃O₅ rt = 2.49 min Calc: 526.1590 (M − H) Found: 526.1580 (M − H) | CDCl₃: 8.21 (s, 1H), 8.13 (d, 1H), 7.98 (dd, 1H), 7.83 (t, 1H), 7.51 (d, 1H), 7.37 (t, 1H), 7.29 (d, 2H), 7.09 (m, 2H), 6.89 (d, 2H), 5.08 (s, 2H), 4.07 (t, 2H), 3.59 (t, 2H), 3.38 (s, 3H), 2.07 (m, 2H) |
| 8 | 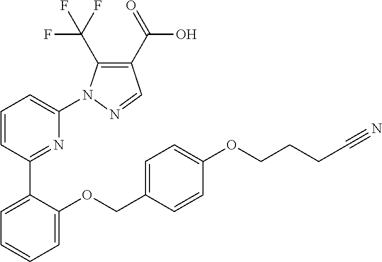 [SMILES: N#CCCCOc1ccc(COc2ccccc2-c2cccc(-n3ncc(C(=O)O)c3C(F)(F)F)n2)cc1] 1-(6-(2-((4-(3- cyanopropoxy)benzyl)oxy)phenyl)pyridin- 2-yl)-5-(trifluoromethyl)-1H-pyrazole- 4-carboxylic acid | C₂₇H₂₁F₃N₄O₄ rt = 2.49 min Calc: 521.1437 (M − H) Found: 521.1401 (M − H) | CDCl₃: 8.22 (s, 1H), 8.13 (d, 1H), 7.97 (dd, 1H), 7.87 (t, 1H), 7.53 (d, 1H), 7.38 (t, 1H), 7.3 (d, 2H), 7.10 (m, 2H), 6.88 (d, 2H), 5.10 (s, 2H), 4.09 (t, 2H), 2.62 (t, 2H), 2.16 (m, 2H) |
| 9 | 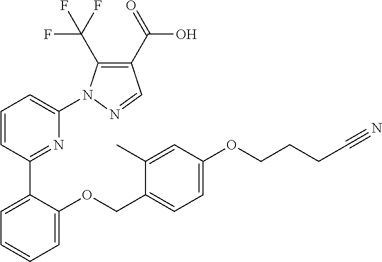 [SMILES: Cc1cc(OCCCC#N)ccc1COc1ccccc1-c1cccc(-n2ncc(C(=O)O)c2C(F)(F)F)n1] 1-(6-(2-((4-(3-cyanopropoxy)-2- methylbenzyl)oxy)phenyl)pyridin-2-yl)- 5-(trifluoromethyl)-1H-pyrazole-4- carboxylic acid | C₂₈H₂₃F₃N₄O₄ rt = 2.65 min Calc: 535.1588 (M − H) Found: 535.1593 (M − H) | CDCl₃: 8.14 (s, 1H), 8.01 (d, 1H), 7.90 (dd, 1H), 7.77 (t, 1H), 7.44 (d, 1H), 7.34 (t, 1H), 7.17 (d, 1H), 7.06 (m, 2H), 6.65 (m, 2H), 4.99 (s, 2H), 4.01 (t, 2H), 2.53 (t, 2H), 2.18 (s, 3H), 2.07 (m, 2H) |
| 10 | 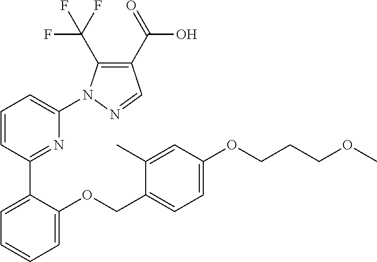 [SMILES: COCCCOc1ccc(COc2ccccc2-c2cccc(-n3ncc(C(=O)O)c3C(F)(F)F)n2)c(C)c1] 1-(6-(2-((4-(3-methoxypropoxy)-2- methylbenzyl)oxy)phenyl)pyridin- 2-yl)-5-(trifluoromethyl)-1H- pyrazole-4-carboxylic acid | C₂₈H₂₆F₃N₃O₅, rt = 2.75 min Calc: 540.1746 (M − H) Found: 540.1758 (M − H) | CDCl₃: 8.21 (s, 1H), 8.09 (d, 1H), 7.99 (dd, 1H), 7.83 (t, 1H), 7.52 (d, 1H), 7.41 (t, 1H), 7.25 (d, 1H), 7.12 (m, 2H), 6.75 (m, 2H), 5.06 (s, 2H), 4.07 (t, 2H), 3.6 (t, 2H), 3.39 (s, 3H), 2.26 (s, 3H), 2.07 (m, 2H) |
| 11 | 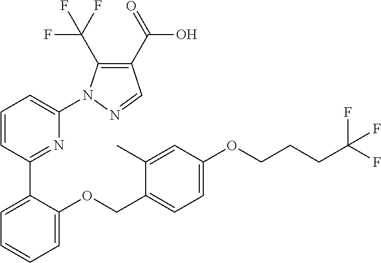 [SMILES: Cc1cc(OCCCC(F)(F)F)ccc1COc1ccccc1-c1cccc(-n2ncc(C(=O)O)c2C(F)(F)F)n1] 1-(6-(2-((2-methyl-4-(4,4,4- trifluorobutoxy)benzyl)oxy)phenyl)pyridin- 2-yl)-5-(trifluoromethyl)-1H-pyrazole- 4-carboxylic acid | C₂₇H₂₀F₇N₃O₄ rt = 2.96 min Calc: 582.1264 (M − H) Found: 582.1259 (M − H) | d6-DMSO: 8.3 (s, 1H), 8.07 (m, 2H), 7.75 (dd, 1H), 7.69 (dd, 1H), 7.45 (m, 2H), 7.34 (d, 1H), 7.12 (t, 1H), 6.89 (dd, 1H), 6.79 (dd, 1H), 5.19 (s, 2H), 4.06 (t, 2H), 2.41 (m, 2H), 1.92 (m, 2H) |
| 12 | 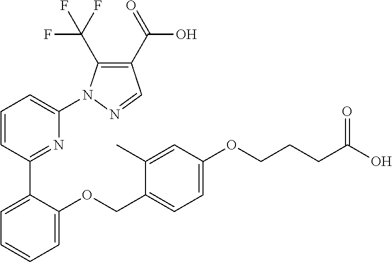 [SMILES: Cc1cc(OCCCC(=O)O)ccc1COc1ccccc1-c1cccc(-n2ncc(C(=O)O)c2C(F)(F)F)n1] 1-(6-(2-((4-(3-carboxypropoxy)-2- methylbenzyl)oxy)phenyl)pyridin- 2-yl)-5-(trifluoromethyl)- 1H-pyrazole-4-carboxylic acid | C₂₈H₂₄F₃N₃O₆ rt = 2.24 min Calc: 554.1488 (M − H) Found: 554.1539 (M − H) | d6-DMSO: 8.29 (s, 1H), 8.05 (m, 2H), 7.74 (dd, 1H), 7.68 (d, 1H), 7.46 (t, 1H), 7.34 (d, 1H), 7.3 (d, 1H), 7.1 (t, 1H), 6.78 (bs, 1H), 6.72 (dd, 1H), 5.13 (s, 2H), 3.95 (t, 2H), 2.37 (t, 2H), 2.2 (s, 3H), 1.92 (m, 2H) |

### Example 13

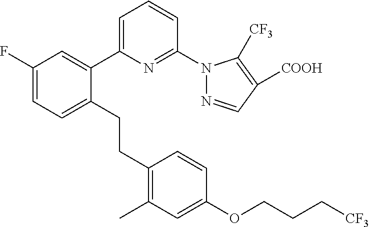 [SMILES: Cc1cc(OCCCC(F)(F)F)ccc1CCc1ccc(F)cc1-c1cccc(-n2ncc(C(=O)O)c2C(F)(F)F)n1]

### 1-(6-(5-Fluoro-2-(2-methyl-4-(4,4,4-trifluorobutoxy)phenethyl)phenyl)pyridin-2-yl)-5-(trifluoromethyl)-1H-pyrazole-4-carboxylic acid

### Intermediate 22: Ethyl 1-(6-(5-fluoro-2-formylphenyl)pyridin-2-yl)-5-(trifluoromethyl)-1H-pyrazole-4-carboxylate

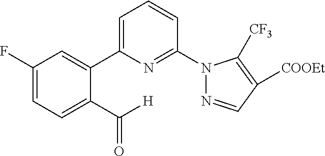 [SMILES: CCOC(=O)c1cnn(-c2cccc(-c3cc(F)ccc3C=O)n2)c1C(F)(F)F]

A solution of ethyl 1-(6-chloropyridin-2-yl)-5-(trifluoromethyl)-1H-pyrazole-4-carboxylate (10 g, 31.3 mmol), (5-fluoro-2-formylphenyl)boronic acid (7.88 g, 46.9 mmol, combi-blocks) and Na₂CO₃ (6.63 g, 62.6 mmol) in 1,2-dimethoxyethane (25 mL) and water (5 mL) was purged with argon for 30 minutes at RT and then tetrakis(triphenylphosphine)palladium(0) (3.61 g, 3.13 mmol) was added. The reaction mixture was heated at 110° C. for 16 hours, then cooled and filtered on a celite pad. The filtrate was diluted with water (30 mL) and extracted with EtOAc (3×20 mL). The organic phase was washed with brine solution (25 mL), dried over anhydrous Na2SO4 and concentrated under reduced pressure. The residue was purified by column chromatography eluting with 10% of EtOAc-Hexane. Collected fractions were concentrated under reduced pressure to afford the title compound (7.5 g, 56.1% yield) as brown solid. LC/MS: rt=3.23 min m/z=408.4 [M+H]⁺

### Intermediate 23: (4-Methoxy-2-methylphenyl)methanol

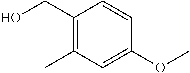

To a solution of 4-methoxy-2-methylbenzoic acid (30 g, 181 mmol), in tetrahydrofuran (1.5 L) stirred under nitrogen at 0° C. was added lithium aluminium hydride (8.22 g, 217 mmol) in portionwise for a period of 30 min. The reaction mixture was stirred at RT for 16 hours. The reaction mixture was slowly quenched with 2N NaOH solution (25 mL) at 0° C. and filtered through celite. The organic layer was concentrated under reduced pressure to give (4-methoxy-2-methylphenyl)methanol (25 g, 164 mmol, 91% yield).

¹H NMR (CDCl₃) δ (ppm): 7.2 (m, 1H), 6.7 (m, 2H), 4.6 (s, 2H), 3.8 (s, 3H), 2.35 (s, 3H).

### Intermediate 24: 1-(Bromomethyl)-4-methoxy-2-methylbenzene (N32190-42-1)

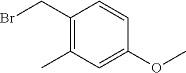

To a stirred solution of (4-methoxy-2-methylphenyl)methanol (11.2 g, 73.6 mmol) in dichloromethane (50 mL) was added at 0° C. phosphorus tribromide (13.88 mL, 147 mmol). The reaction mixture was allowed to warm to RT and stirred for 2 hours. The reaction mixture was quenched with ice cold water (50 mL) and extracted with dichloromethane (2×20 mL) and washed with a NaHCO₃ solution (30 mL). The organic layer was concentrated under reduced pressure to give 1-(bromomethyl)-4-methoxy-2-methylbenzene (11.2 g, 52.1 mmol, 70.8% yield). The compound was used in the next step without further purification.

### Intermediate 25: Diethyl 4-methoxy-2-methyl benzylphosphonate

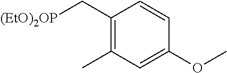

A solution of 1-(bromomethyl)-4-methoxy-2-methylbenzene (10 g, 46.5 mmol) and triethyl phosphite (10.16 mL, 58.1 mmol) in 1,4-dioxane (2.5 mL) was heated at 100° C. for 20 hours. The reaction mixture was quenched with water (10 mL) and the aqueous layer was extracted with EtOAc (2×10 mL). Combined organic layers were dried over anhydrous Na₂SO₄ and concentrated under reduced pressure to afford diethyl 4-methoxy-2-methylbenzylphosphonate (10 g, 36.7 mmol, 79% yield) as colorless liquid.

¹H NMR (CDCl₃) δ (ppm): 7.2 (m, 1H), 6.7 (m, 2H), 4 (m, 4H), 3.8 (s, 3H), 3.1 (d, 2H), 2.35 (s, 3H), 1.2 (m, 6H).

### Intermediate 26: (E)-Ethyl 1-(6-(5-fluoro-2-(4-methoxy-2-methylstyryl)phenyl)pyridin-2-yl)-5-(trifluoromethyl)-1H-pyrazole-4-carboxylate

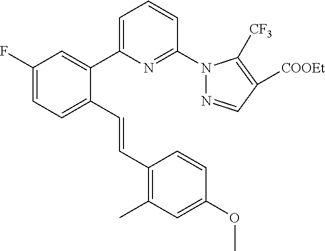 [SMILES: CCOC(=O)c1cnn(-c2cccc(-c3cc(F)ccc3/C=C/c3ccc(OC)cc3C)n2)c1C(F)(F)F]

To a suspension of NaH (0.982 g, 24.55 mmol) in tetrahydrofuran (50 mL) stirred under nitrogen at 0° C. was added a solution of diethyl 4-methoxy-2-methylbenzylphosphonate (5.01 g, 18.41 mmol) in tetrahydrofuran (50 mL) dropwise during 5 min then added ethyl 1-(6-(5-fluoro-2-formylphenyl)pyridin-2-yl)-5-(trifluoromethyl)-1H-pyrazole-4-carboxylate (5 g, 12.28 mmol). Reaction mixture was stirred at RT for 16 hours. The reaction mixture was diluted with water (10 mL) and extracted with EtOAc (3×20 mL). The organic phase was washed with brine solution (25 mL), dried over anhydrous Na2SO4 and concentrated under reduced pressure. The residue was purified by column chromatography eluting with 20% EtOAc in hexane. The collected fractions were concentrated under reduced pressure to afford (E)-ethyl 1-(6-(5-fluoro-2-(4-methoxy-2-methylstyryl)phenyl)pyridin-2-yl)-5-(trifluoromethyl)-1H-pyrazole-4-carboxylate (2.4 g, 34.5% yield) as colorless semi-solid. LC/MS: rt=3.29 min m/z=526.30 [M+H]⁺

### Intermediate 27: Ethyl 1-(6-(5-fluoro-2-(4-methoxy-2-methylphenethyl)phenyl)pyridin-2-yl)-5-(trifluoromethyl)-1H-pyrazole-4-carboxylate

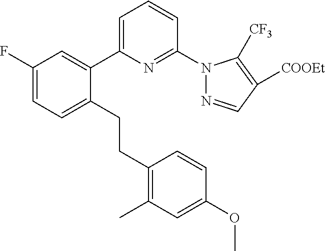 [SMILES: CCOC(=O)c1cnn(-c2cccc(-c3cc(F)ccc3CCc3ccc(OC)cc3C)n2)c1C(F)(F)F]

To a solution of (E)-ethyl 1-(6-(5-fluoro-2-(4-methoxy-2-methylstyryl)phenyl)pyridin-2-yl)-5-(trifluoromethyl)-1H-pyrazole-4-carboxylate (2.4 g, 4.57 mmol) in methanol (50 mL) stirred at room temp was added Pd/C (0.486 g, 0.457 mmol) in methanol (50 mL). The reaction mixture was stirred at RT under Hydrogen pressure (30 Psi) for 2 hours. The reaction mixture was filtered on a celite bed, and the filtrate was concentrated under reduced pressure to afford ethyl 1-(6-(5-fluoro-2-(4-methoxy-2-methylphenethyl)phenyl)pyridin-2-yl)-5-(trifluoromethyl)-1H-pyrazole-4-carboxylate (2.1 g, 3.68 mmol, 80% yield). LC/MS: rt=3.24 min m/z=528.31 [M+H]⁺.

### Intermediate 28: Ethyl 1-(6-(5-fluoro-2-(4-hydroxy-2-methylphenethyl)phenyl)pyridin-2-yl)-5-(trifluoromethyl)-1H-pyrazole-4-carboxylate

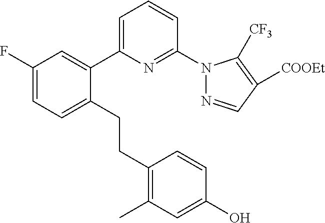 [SMILES: CCOC(=O)c1cnn(-c2cccc(-c3cc(F)ccc3CCc3ccc(O)cc3C)n2)c1C(F)(F)F]

To a solution of ethyl 1-(6-(5-fluoro-2-(4-methoxy-2-methylphenethyl)phenyl)pyridin-2-yl)-5-(trifluoromethyl)-1H-pyrazole-4-carboxylate (6.6 g, 12.51 mmol) in dichloromethane (50 mL) stirred under nitrogen, was added boron tribromide (1.419 mL, 15.01 mmol). The reaction mixture was stirred at RT for 3 hours and then diluted with water (50 mL) and extracted with dichloromethane (3×50 mL). The organic phase was washed with Na₂CO₃ solution (50 mL), dried over anhydrous Na2SO4 and concentrated under reduced pressure to afford ethyl 1-(6-(5-fluoro-2-(4-hydroxy-2-methylphenethyl)phenyl)pyridin-2-yl)-5-(trifluoromethyl)-1H-pyrazole-4-carboxylate (4 g, 56.1% yield). LC/MS: rt=4.03 min m/z=514.20 [M+H]⁺

### Intermediate 29: Ethyl 1-(6-(5-fluoro-2-(2-methyl-4-(4,4,4-trifluorobutoxy)phenethyl)phenyl)pyridin-2-yl)-5-(trifluoromethyl)-1H-pyrazole-4-carboxylate

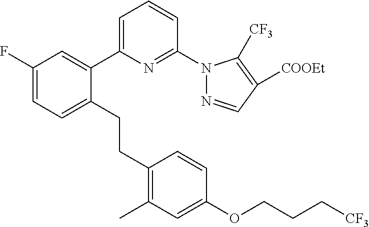 [SMILES: CCOC(=O)c1cnn(-c2cccc(-c3cc(F)ccc3CCc3ccc(OCCCC(F)(F)F)cc3C)n2)c1C(F)(F)F]

To a solution of ethyl 1-(6-(5-fluoro-2-(4-hydroxy-2-methylphenethyl)phenyl)pyridin-2-yl)-5-(trifluoromethyl)-1H-pyrazole-4-carboxylate (200 mg, 0.389 mmol) in DMF (15 mL) stirred under nitrogen, and at 0° C., were added K₂CO₃ (108 mg, 0.779 mmol) and 4-bromo-1,1,1-trifluorobutane (156 mg, 0.818 mmol). The reaction mixture was stirred at 100° C. for 16 hours, then cooled and diluted with water (10 mL). After extraction with EtOAc (3×20 mL), the organic phase was washed with brine solution (25 mL), dried over anhydrous Na₂SO₄ and concentrated under reduced pressure to afford ethyl 1-(6-(5-fluoro-2-(2-methyl-4-(4,4,4-trifluorobutoxy)phenethyl)phenyl)pyridin-2-yl)-5-(trifluoromethyl)-1H-pyrazole-4-carboxylate (200 mg, 0.282 mmol, 72.5% yield). LC/MS: rt=3.39 min m/z=624.34 [M+H]⁺.

### Final compound 1-(6-(5-Fluoro-2-(2-methyl-4-(4,4,4-trifluorobutoxy)phenethyl)phenyl)pyridin-2-yl)-5-(trifluoromethyl)-1H-pyrazole-4-carboxylic acid

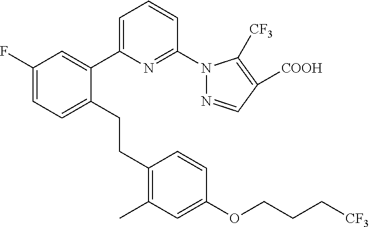 [SMILES: Cc1cc(OCCCC(F)(F)F)ccc1CCc1ccc(F)cc1-c1cccc(-n2ncc(C(=O)O)c2C(F)(F)F)n1]

To a solution of Intermediate 29 ethyl 1-(6-(5-fluoro-2-(2-methyl-4-(4,4,4-trifluorobutoxy)phenethyl)phenyl)pyridin-2-yl)-5-(trifluoromethyl)-1H-pyrazole-4-carboxylate (200 mg, 0.321 mmol) in EtOH (5 mL) and water (0.7 mL) was added NaOH (38.5 mg, 0.962 mmol) at 0° C. and the reaction mixture was allowed to stir at room temperature for 16 hours and then was concentrated under reduced pressure. The residue was dissolved in cold water (0.5 mL) and acidified with saturated citric acid solution up to pH 4. After extraction with dichloromethane (3×15 mL), the combined organic phases were washed with water (2×15 mL), dried over anhydrous Na₂SO₄ and concentrated under reduced pressure. The residue was purified by prep-HPLC (method B). Fractions were collected and lyophilized to afford the 1-(6-(5-fluoro-2-(2-methyl-4-(4,4,4-trifluorobutoxy)phenethyl)phenyl)pyridin-2-yl)-5-(trifluoromethyl)-1H-pyrazole-4-carboxylic acid (66.3 mg, 34.7% yield) as a brown solid.

¹H NMR (d⁶-DMSO) δ (ppm): 13.8 (s, 1H), 8.32 (s, 1H), 8.21 (t, 1H), 7.82 (d, 1H), 7.7 (d, 1H), 7.42 (m, 1H), 7.24 (m, 2H), 6.63 (m, 2H), 6.53 (m, 1H), 3.94 (t, 2H), 2.81 (m, 2H), 2.56 (m, 2H), 2.42-2.36 (m, 2H), 1.93 (s, 3H), 1.88 (m, 2H)

LC/MS: rt=3.06 min m/z=596.17 [M+H]⁺.

### Example 14

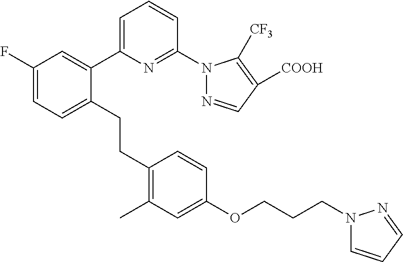 [SMILES: Cc1cc(OCCCn2cccn2)ccc1CCc1ccc(F)cc1-c1cccc(-n2ncc(C(=O)O)c2C(F)(F)F)n1]

### 1-(6-(2-(4-(3-(1H-pyrazol-1-yl)propoxy)-2-methylphenethyl)-5-fluorophenyl)pyridin-2-yl)-5-(trifluoromethyl)-1H-pyrazole-4-carboxylic acid

### Intermediate 30: 3-(1H-Pyrazol-1-yl)propan-1-ol

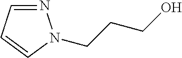

A solution of 1H-pyrazole (500 mg, 7.34 mmol, Aldrich), 3-bromopropan-1-ol (1531 mg, 11.02 mmol, Aldrich) and cesium carbonate (4786 mg, 14.69 mmol) in N,N-dimethylformamide (10 mL) was stirred at 80° C. under nitrogen for 16 hours. The reaction mixture was diluted with water (10 mL) and extracted with diethyl ether (3×20 mL) and washed with brine solution (25 mL). The organic layer was separated, dried over anhydrous Na₂SO₄ and concentrated under reduced pressure to afford 3-(1H-pyrazol-1-yl)propan-1-ol (450 mg, 3.57 mmol, 48.6% yield) as colorless liquid.

¹H NMR (d⁶-DMSO) δ (ppm): 7.7 (s, 1H), 7.4 (s, 1H), 6.2 (s, 1H), 4.5 (t, 1H), 4.1 (t, 2H), 3.35 (m, 2H), 1.9 (m, 2H).

### Intermediate 31: 3-(1H-Pyrazol-1-yl)propyl methanesulfonate

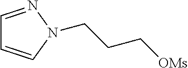 [SMILES: CS(=O)(=O)OCCCn1cccn1]

A solution of 3-(1H-pyrazol-1-yl)propan-1-ol (450 mg, 3.57 mmol), triethylamine (0.497 mL, 3.57 mmol) and mesyl chloride (0.278 mL, 3.57 mmol) in dichloromethane (10 mL) was stirred under nitrogen at RT for 2 hours. The reaction mixture was filtered and the filtrate was concentrated under reduced pressure to afford 3-(1H-pyrazol-1-yl)propyl methanesulfonate (700 mg, 3.43 mmol, 96% yield). LC/MS: rt=1.35 min m/z=205.05 [M+H]⁺

### Intermediate 32: Ethyl 1-(6-(2-(4-(3-(1H-pyrazol-1-yl)propoxy)-2-methylphenethyl)-5-fluorophenyl)pyridin-2-yl)-5-(trifluoromethyl)-1H-pyrazole-4-carboxylate

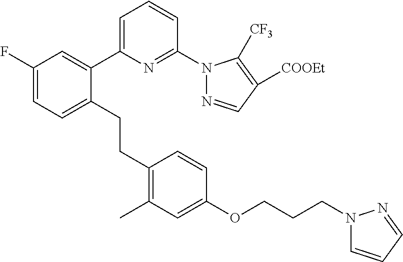 [SMILES: CCOC(=O)c1cnn(-c2cccc(-c3cc(F)ccc3CCc3ccc(OCCCn4cccn4)cc3C)n2)c1C(F)(F)F]

A mixture of 3-(1H-pyrazol-1-yl)propyl methanesulfonate (700 mg, 3.43 mmol), cesium carbonate (2233 mg, 6.85 mmol) and ethyl 1-(6-(5-fluoro-2-(4-hydroxy-2-methylphenethyl)phenyl)pyridin-2-yl)-5-(trifluoromethyl)-1H-pyrazole-4-carboxylate (2.64 g, 5.14 mmol) in acetonitrile (10 mL) was stirred under nitrogen. The reaction mixture was stirred at 70° C. for 16 hours and then diluted with water (10 mL) and extracted with EtOAc (3×20 mL). The organic phase was washed with brine solution (25 mL), dried over anhydrous Na2SO4 and concentrated under reduced pressure. The residue was purified by column chromatography eluting with 10% EtOAc in hexane. Collected fractions were concentrated under reduced pressure to afford ethyl 1-(6-(2-(4-(3-(1H-pyrazol-1-yl)propoxy)-2-methylphenethyl)-5-fluorophenyl)pyridin-2-yl)-5-(trifluoromethyl)-1H-pyrazole-4-carboxylate, (180 mg, 0.29 mmol, 6.94% yield). LC/MS: rt=4.28 min m/z=622.22 [M+H]⁺

### Final compound 1-(6-(2-(4-(3-(1H-pyrazol-1-yl)propoxy)-2-methylphenethyl)-5-fluorophenyl)pyridin-2-yl)-5-(trifluoromethyl)-1H-pyrazole-4-carboxylic acid

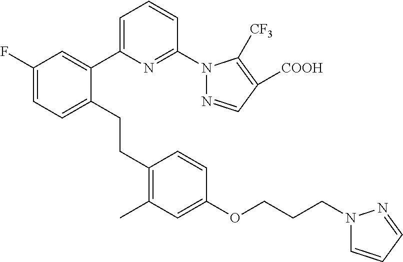 [SMILES: Cc1cc(OCCCn2cccn2)ccc1CCc1ccc(F)cc1-c1cccc(-n2ncc(C(=O)O)c2C(F)(F)F)n1]

To a solution of Intermediate 32 ethyl 1-(6-(2-(4-(3-(1H-pyrazol-1-yl)propoxy)-2-methylphenethyl)-5-fluorophenyl)pyridin-2-yl)-5-(trifluoromethyl)-1H-pyrazole-4-carboxylate (180 mg, 0.290 mmol) in ethanol (5 mL) was added lithium hydroxide (20.80 mg, 0.869 mmol) in water (2 mL). The reaction mixture was stirred at RT for 16 hours, and then was diluted with water (10 mL) and acidified with citric acid solution up to pH 4. After extraction with EtOAc (3×20 mL), the organic phase was washed with brine solution (25 mL), dried over anhydrous Na2SO4, filtered and concentrated under reduced pressure. The residue was purified by prep-HPLC (method C). Fractions were collected and concentrated under reduced pressure to remove acetonitrile, the aqueous layer was extracted with EtOAc (2×50 mL) and the organic phase was washed with brine solution (25 mL), dried over anhydrous Na2SO4 and concentrated under reduced pressure. The residue was washed with n-pentane (3×5 mL) and dried under high vacuo to afford 1-(6-(2-(4-(3-(1H-pyrazol-1-yl)propoxy)-2-methylphenethyl)-5-fluorophenyl)pyridin-2-yl)-5-(trifluoromethyl)-1H-pyrazole-4-carboxylic acid (38.6 mg, 21.73% yield) as off white solid. LC/MS: rt=2.77 min m/z=594.26 [M+H]⁺.

¹H NMR (d⁶-DMSO), δ (ppm): 13.4 (s, 1H), 8.32 (s, 1H), 8.2 (t, 1H), 7.82 (d, 1H), 7.69 (m, 2H), 7.43 (m, 2H), 7.24 (m, 2H), 6.61 (m, 2H), 6.5 (m, 1H), 6.22 (t, 1H), 4.25 (t, 2H), 3.83 (t, 2H), 2.81 (m, 2H), 2.56 (m, 2H), 2.16 (m, 2H), 1.93 (s, 3H).

### Example 15

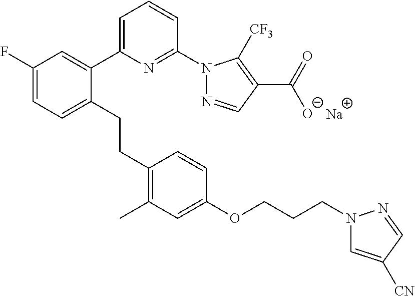 [SMILES: Cc1cc(OCCCn2cc(C#N)cn2)ccc1CCc1ccc(F)cc1-c1cccc(-n2ncc(C(=O)[O-])c2C(F)(F)F)n1 ; [Na+]]

### 1-(6-(2-(4-(3-(4-cyano-1H-pyrazol-1-yl)propoxy)-2-methylphenethyl)-5-fluorophenyl)pyridin-2-yl)-5-(trifluoromethyl)-1H-pyrazole-4-carboxylic acid, sodium salt

### Intermediate 33: Ethyl 1-(6-(2-(4-(3-bromopropoxy)-2-methylphenethyl)-5-fluorophenyl)pyridin-2-yl)-5-(trifluoromethyl)-1H-pyrazole-4-carboxylate

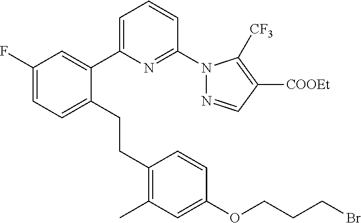 [SMILES: CCOC(=O)c1cnn(-c2cccc(-c3cc(F)ccc3CCc3ccc(OCCCBr)cc3C)n2)c1C(F)(F)F]

To a solution of commercially available of 1,3-dibromopropane (1.966 g, 9.74 mmol, Acros Organics) and ethyl 1-(6-(5-fluoro-2-(4-hydroxy-2-methylphenethyl)phenyl)pyridin-2-yl)-5-(trifluoromethyl)-1H-pyrazole-4-carboxylate, (1 g, 1.947 mmol) in acetonitrile (2 mL) was added Cs₂CO₃ (0.761 g, 2.337 mmol). The reaction mixture was stirred under nitrogen at RT for 16 hours. The reaction mixture was concentrated and the crude was dissolved in water (50 mL) and the product was extracted with EtOAc (3×35 mL). The combined organic phase was dried over anhydrous Na₂SO₄, filtered and concentrated under reduced pressure to obtain crude product. Purification by column chromatography eluting with 10-12% EtOAc in hexane afforded ethyl 1-(6-(2-(4-(3-bromopropoxy)-2-methylphenethyl)-5-fluorophenyl)pyridin-2-yl)-5-(trifluoromethyl)-1H-pyrazole-4-carboxylate (800 mg, 0.877 mmol, 45.0% yield) as a brown gum. LCMS: rt=4.56 min, m/z=634.12-636.13 [M+H]⁺

### Intermediate 34: Ethyl 1-(6-(2-(4-(3-(4-cyano-1H-pyrazol-1-yl)propoxy)-2-methylphenethyl)-5-fluorophenyl)pyridin-2-yl)-5-(trifluoromethyl)-1H-pyrazole-4-carboxylate

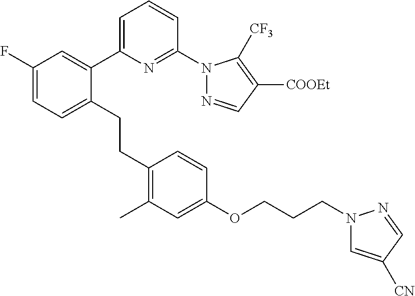 [SMILES: CCOC(=O)c1cnn(-c2cccc(-c3cc(F)ccc3CCc3ccc(OCCCn4cc(C#N)cn4)cc3C)n2)c1C(F)(F)F]

To a solution of ethyl 1-(6-(2-(4-(3-bromopropoxy)-2-methylphenethyl)-5-fluorophenyl)pyridin-2-yl)-5-(trifluoromethyl)-1H-pyrazole-4-carboxylate (250 mg, 0.394 mmol) and 1H-pyrazole-4-carbonitrile (44.0 mg, 0.473 mmol) in acetonitrile (20 mL) was added cesium carbonate (193 mg, 0.591 mmol). The reaction mixture was stirred at 80° C. for 16 hours and then concentrated under reduced pressure. EtOAc (50 mL) was added to the residue and the precipitate was filtered off through a celite pad and washed with EtOAc (2×25 mL). The filtrate was concentrated under reduced pressure. The residue was purified by column chromatography eluting with 12-14% EtOAc-hexane to afford ethyl 1-(6-(2-(4-(3-(4-cyano-1H-pyrazol-1-yl)propoxy)-2-methylphenethyl)-5-fluorophenyl)pyridin-2-yl)-5-(trifluoromethyl)-1H-pyrazole-4-carboxylate (230 mg, 87% yield) as a gummy product. LCMS: rt=4.29 min, m/z=647.35 [M+H]⁺

### 1-(6-(2-(4-(3-(4-cyano-1H-pyrazol-1-yl)propoxy)-2-methylphenethyl)-5-fluorophenyl)pyridin-2-yl)-5-(trifluoromethyl)-1H-pyrazole-4-carboxylic acid

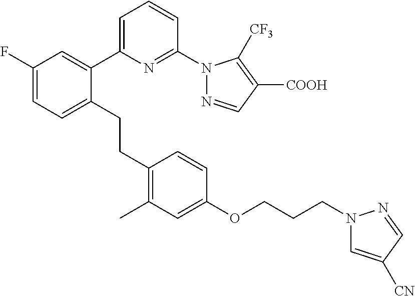 [SMILES: Cc1cc(OCCCn2cc(C#N)cn2)ccc1CCc1ccc(F)cc1-c1cccc(-n2ncc(C(=O)O)c2C(F)(F)F)n1]

To a solution of ethyl 1-(6-(2-(4-(3-(4-cyano-1H-pyrazol-1-yl)propoxy)-2-methylphenethyl)-5-fluorophenyl)pyridin-2-yl)-5-(trifluoromethyl)-1H-pyrazole-4-carboxylate (230 mg, 0.356 mmol) in ethanol (15 mL) was added sodium hydroxide (28.5 mg, 0.711 mmol) in water (1 mL). Reaction mixture was stirred at RT for 16 hours and then concentrated under reduced pressure. The residue was dissolved in cold water (1 mL) and acidified with saturated citric acid solution up to pH 4. After extraction with dichloromethane (3×15 mL), the combined organic phase was dried over anhydrous Na₂SO₄ and concentrated under reduced pressure. The residue was purified by prep-HPLC (method C). The fractions were concentrated under reduced pressure to remove volatile solvent, acidified with diluted acetic acid (pH 5) and then extracted with ethyl acetate (3×20 mL). The organic phase was dried over anhydrous Na₂SO₄, filtered and concentrated under reduced pressure to afford 1-(6-(2-(4-(3-(4-cyano-1H-pyrazol-1-yl)propoxy)-2-methylphenethyl)-5-fluorophenyl)pyridin-2-yl)-5-(trifluoromethyl)-1H-pyrazole-4-carboxylic acid (160 mg, 0.258 mmol, 72.5% yield). LCMS: rt=3.19 min, m/z=619.32 [M+H]

¹H NMR (CDCl₃), δ (ppm): 8.2 (s, 1H), 7.95 (m, 1H), 7.8 (m, 2H), 7.6 (m, 1H), 7.45 (m, 1H), 7.25 (m, 1H), 7.1 (m, 2H), 6.65 (m, 1H), 6.5 (m, 2H), 4.4 (t, 2H), 3.85 (t, 2H), 2.9 (m, 2H), 2.7 (m, 2H), 2.3 (m, 2H), 2 (s, 3H).

### Final compound 1-(6-(2-(4-(3-(4-Cyano-1H-pyrazol-1-yl)propoxy)-2-methylphenethyl)-5-fluorophenyl)pyridin-2-yl)-5-(trifluoromethyl)-1H-pyrazole-4-carboxylic acid, sodium salt

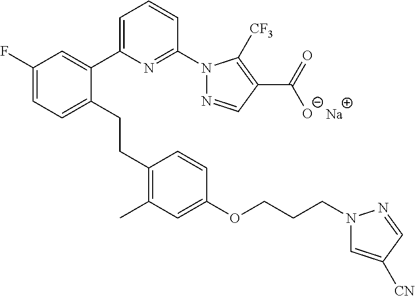 [SMILES: Cc1cc(OCCCn2cc(C#N)cn2)ccc1CCc1ccc(F)cc1-c1cccc(-n2ncc(C(=O)O)c2C(F)(F)F)n1 ; [Na+]]

To a solution of the previously made 1-(6-(2-(4-(3-(4-cyano-1H-pyrazol-1-yl)propoxy)-2-methylphenethyl)-5-fluorophenyl)pyridin-2-yl)-5-(trifluoromethyl)-1H-pyrazole-4-carboxylic acid (48 mg, 0.078 mmol) in water (5 mL) and was added sodium hydroxide (3.10 mg, 0.078 mmol) in water (5 mL). The reaction mixture was stirred at RT for 20 min. Then the solution was lyophilized for 20 hours to afford 1-(6-(2-(4-(3-(4-cyano-1H-pyrazol-1-yl)propoxy)-2-methylphenethyl)-5-fluorophenyl)pyridin-2-yl)-5-(trifluoromethyl)-1H-pyrazole-4-carboxylic acid, sodium salt (44.3 mg, 0.069 mmol, 89% yield) as an off white solid (hygroscopic). LCMS: rt=2.77 min, m/z=617.37 [M−H]⁻

¹H NMR (d⁶-DMSO), δ (ppm): 8.6 (s, 1H), 8.1 (t, 1H), 8.05 (s, 1H), 7.75 (s, 1H), 7.7 (d, 1H), 7.55 (d, 1H), 7.41 (m, 1H), 7.22 (m, 2H), 6.65 (d, 1H), 6.57 (m, 1H), 6.51 (m, 1H), 4.32 (t, 2H), 3.87 (t, 2H), 2.83 (m, 2H), 2.55 (m, 2H), 2.19 (m, 2H), 1.92 (s, 3H)

### Example 16

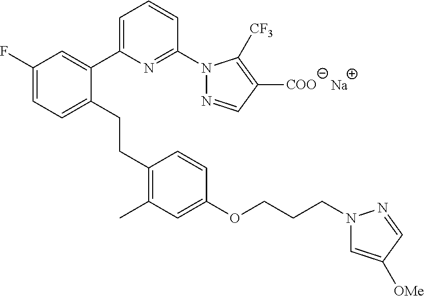

### 1-(6-(5-fluoro-2-(4-(3-(4-methoxy-1H-pyrazol-1-yl)propoxy)-2-methyl phenethyl)phenyl)pyridin-2-yl)-5-(trifluoromethyl)-1H-pyrazole-4-carboxylic acid, sodium salt

### Intermediate 35: Ethyl 1-(6-(5-fluoro-2-(4-(3-(4-methoxy-1H-pyrazol-1-yl)propoxy)-2-methylphenethyl)phenyl)pyridin-2-yl)-5-(trifluoromethyl)-1H-pyrazole-4-carboxylate

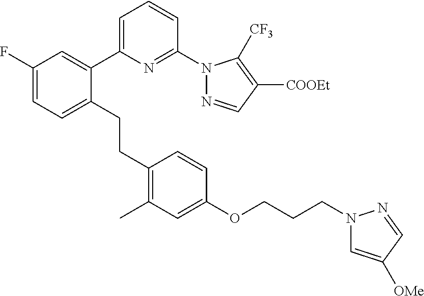 [SMILES: CCOC(=O)c1cnn(-c2cccc(-c3cc(F)ccc3CCc3ccc(OCCCn4cc(OC)cn4)cc3C)n2)c1C(F)(F)F]

To a solution of ethyl 1-(6-(2-(4-(3-bromopropoxy)-2-methylphenethyl)-5-fluorophenyl)pyridin-2-yl)-5-(trifluoromethyl)-1H-pyrazole-4-carboxylate (see Intermediate 12 for prep) (200 mg, 0.315 mmol) in N,N-dimethylformamide (3 mL) was added cesium carbonate (154 mg, 0.473 mmol) and 4-methoxy-1H-pyrazole (46.4 mg, 0.473 mmol) and the reaction mixture was heated at 70° C. for 16 hours. The reaction mixture was cooled to room temperature, filtered through celite pad and the solid was washed with EtOAc (3×25 mL). The filtrate was concentrated under reduced pressure. The crude was purified by column chromatography eluting with 25-26% of EtOAc-hexane to afford ethyl 1-(6-(5-fluoro-2-(4-(3-(4-methoxy-1H-pyrazol-1-yl)propoxy)-2-methylphenethyl)phenyl)pyridin-2-yl)-5-(trifluoromethyl)-1H-pyrazole-4-carboxylate (60 mg, 0.069 mmol, 21.79% yield) as a gum. LC/MS: rt=4.29 min, m/z=652.32 [M+H]⁺

### 1-(6-(5-fluoro-2-(4-(3-(4-methoxy-1H-pyrazol-1-yl)propoxy)-2-methylphenethyl)phenyl)pyridin-2-yl)-5-(trifluoromethyl)-1H-pyrazole-4-carboxylic acid

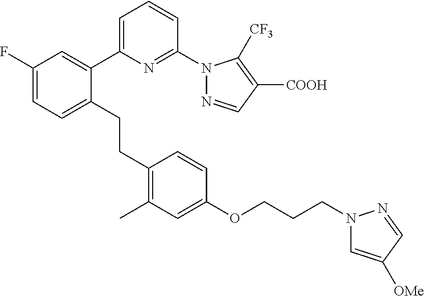 [SMILES: COc1cnn(CCCOc2ccc(CCc3ccc(F)cc3-c3cccc(-n4ncc(C(=O)O)c4C(F)(F)F)n3)c(C)c2)c1]

To a solution of ethyl 1-(6-(5-fluoro-2-(4-(3-(4-methoxy-1H-pyrazol-1-yl)propoxy)-2-methylphenethyl)phenyl)pyridin-2-yl)-5-(trifluoromethyl)-1H-pyrazole-4-carboxylate (60 mg, 0.092 mmol) in ethanol (15 mL) was added sodium hydroxide (7.37 mg, 0.184 mmol) in water (1 mL). The reaction mixture was stirred at RT for 16 hour and then concentrated under reduced pressure. The residue was dissolved in cold water (1 mL) and acidified with saturated citric acid solution up to pH 4. After extraction with dichloromethane (3×15 mL), the combined organic phase was dried over anhydrous Na₂SO₄ and concentrated under reduced pressure. Two batches of the crude residue were purified by prep-HPLC (method A). Fractions were collected and evaporated under vacuo. Crude was acidified with diluted acetic acid (pH 5). Product was extracted with EtOAc (3×15 mL), combined organic phase was dried over anhydrous Na₂SO₄, filtered and concentrated under reduced pressure to afford desired compound 1-(6-(5-fluoro-2-(4-(3-(4-methoxy-1H-pyrazol-1-yl)propoxy)-2-methylphenethyl)phenyl)pyridin-2-yl)-5-(trifluoromethyl)-1H-pyrazole-4-carboxylic acid (130 mg, 0.206 mmol). LC/MS: rt=3.17 min, m/z=624.29 [M+H]⁺

¹H NMR (CDCl₃), δ (ppm): 8.1 (s, 1H), 7.95 (t, 1H), 7.55 (m, 2H), 7.35 (s, 1H), 7.3 (m, 1H), 7.25 (m, 1H), 7.1 (m, 2H), 6.55 (m, 2H), 6.45 (m, 1H), 4.3 (t, 2H), 3.85 (t, 2H), 3.75 (s, 3H), 2.95 (m, 2H), 2.75 (m, 2H), 2.2 (m, 2H), 2.05 (s, 3H).

### Final compound 1-(6-(5-Fluoro-2-(4-(3-(4-methoxy-1H-pyrazol-1-yl)propoxy)-2-methylphenethyl)phenyl)pyridin-2-yl)-5-(trifluoromethyl)-1H-pyrazole-4-carboxylic acid, sodium salt

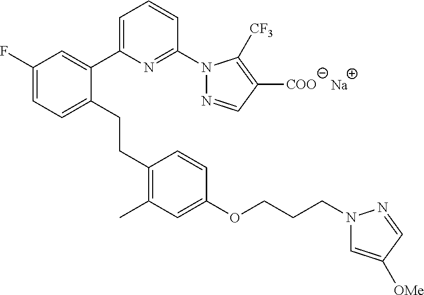

To a solution of previously made 1-(6-(5-fluoro-2-(4-(3-(4-methoxy-1H-pyrazol-1-yl)propoxy)-2-methylphenethyl)phenyl)pyridin-2-yl)-5-(trifluoromethyl)-1H-pyrazole-4-carboxylic acid (130 mg, 0.208 mmol) in water (5 mL) was added sodium hydroxide (8.34 mg, 0.208 mmol) in water (5 mL). The reaction mixture was stirred at RT for 20 min. Then the solution was transferred to lyophilization flask and lyophilized for 20 hours to afford 1-(6-(5-fluoro-2-(4-(3-(4-methoxy-1H-pyrazol-1-yl)propoxy)-2-methylphenethyl)phenyl)pyridin-2-yl)-5-(trifluoromethyl)-1H-pyrazole-4-carboxylic acid, sodium salt (122 mg, 0.184 mmol, 89% yield) as off-white solid (hygroscopic product). LCMS: rt=2.75 min, m/z=624.38 [M+H]⁺

¹H NMR (d⁶-DMSO), δ (ppm): 8.1 (t, 1H), 7.76 (d, 1H), 7.7 (dd, 1H), 7.54 (dd, 1H), 7.47 (d, 1H), 7.4 (dd, 1H), 7.23 (m, 2H), 7.17 (m, 1H), 6.66 (d, 1H), 6.58 (d, 1H), 6.52 (dd, 1H), 4.15 (t, 2H), 3.84 (t, 2H), 3.62 (s, 3H), 2.83 (m, 2H), 2.54 (m, 2H), 2.12 (m, 2H), 2.1 (s, 3H).

### Example 17

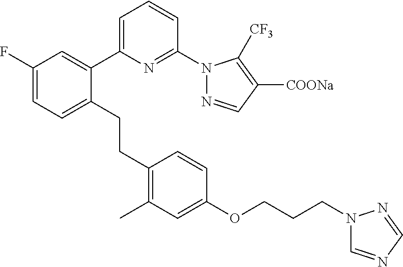

### 1-(6-(2-(4-(3-(1H-1,2,4-triazol-1-yl)propoxy)-2-methylphenethyl)-5-fluorophenyl)pyridin-2-yl)-5-(trifluoromethyl)-1H-pyrazole-4-carboxylic acid, sodium salt

### Intermediate 36: Ethyl 1-(6-(2-(4-(3-(1H-1,2,4-triazol-1-yl)propoxy)-2-methylphenethyl)-5-fluorophenyl)pyridin-2-yl)-5-(trifluoromethyl)-1H-pyrazole-4-carboxylate

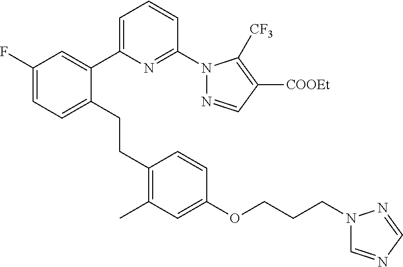 [SMILES: CCOC(=O)c1cnn(-c2cccc(-c3cc(F)ccc3CCc3ccc(OCCCn4cncn4)cc3C)n2)c1C(F)(F)F]

A mixture of ethyl 1-(6-(2-(4-(3-bromopropoxy)-2-methylphenethyl)-5-fluorophenyl)pyridin-2-yl)-5-(trifluoromethyl)-1H-pyrazole-4-carboxylate (250 mg, 0.394 mmol) and triazole sodium salt (53.8 mg, 0.591 mmol) in DMF (3 mL) was stirred at 70° C. for 20 hours, then cooled and diluted with cold water. After extraction with EtOAc (6×25 mL), the combined organic phase was washed with cold water (3×25 mL), dried over anhydrous Na₂SO₄ and concentrated under reduced pressure. The residue was purified by column chromatography eluting with 65-70% EtOAc-hexane to afford ethyl 1-(6-(2-(4-(3-(1H-1,2,4-triazol-1-yl) propoxy)-2-methylphenethyl)-5-fluorophenyl)pyridin-2-yl)-5-(trifluoromethyl)-1H-pyrazole-4-carboxylate (180 mg, 73.4% yield). LC/MS: rt=4.07 min, m/z=623.22 [M+H]⁺

### 1-(6-(2-(4-(3-(1H-1,2,4-Triazol-1-yl)propoxy)-2-methylphenethyl)-5-fluorophenyl)pyridin-2-yl)-5-(trifluoromethyl)-1H-pyrazole-4-carboxylic acid

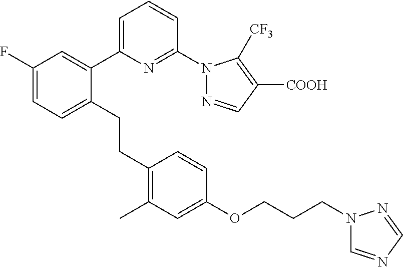 [SMILES: Cc1cc(OCCCn2cncn2)ccc1CCc1ccc(F)cc1-c1cccc(-n2ncc(C(=O)O)c2C(F)(F)F)n1]

To a solution of ethyl 1-(6-(2-(4-(3-(1H-1,2,4-triazol-1-yl)propoxy)-2-methylphenethyl)-5-fluorophenyl)pyridin-2-yl)-5-(trifluoromethyl)-1H-pyrazole-4-carboxylate (300 mg, 0.482 mmol) in water (1 mL) and EtOH (15 mL) was added sodium hydroxide (38.5 mg, 0.964 mmol) in water (1 mL). The reaction mixture was stirred at room temperature for 16 hours and then concentrated under reduced pressure. The residue was dissolved in cold water (1 mL) and acidified with saturated citric acid solution up to pH 4. The precipitate was filtered, washed with water (3×10 mL) and dried under high vacuo to afford 1-(6-(2-(4-(3-(1H-1,2,4-Triazol-1-yl)propoxy)-2-methylphenethyl)-5-fluorophenyl)pyridin-2-yl)-5-(trifluoromethyl)-1H-pyrazole-4-carboxylic acid (170 mg, 59.3% yield) as off white solid. LCMS: rt=2.8 min, m/z=595.32 [M+H]⁺

¹H NMR (CDCl₃), δ (ppm): 8.15 (m, 2H), 8.05 (s, 1H), 7.95 (t, 1H), 7.6 (dd, 1H), 7.5 (dd, 1H), 7.25 (m, 1H), 7.15 (m, 1H), 7.05 (m, 1H), 6.65 (m, 1H), 6.55 (d, 1H), 6.45 (m, 1H), 4.45 (t, 2H), 3.8 (t, 2H), 2.95 (m, 2H), 2.7 (m, 2H), 2.3 (m, 2H), 2.05 (s, 3H).

### Final compound 1-(6-(2-(4-(3-(1H-1,2,4-Triazol-1-yl)propoxy)-2-methylphenethyl)-5-fluorophenyl)pyridin-2-yl)-5-(trifluoromethyl)-1H-pyrazole-4-carboxylic acid, sodium salt

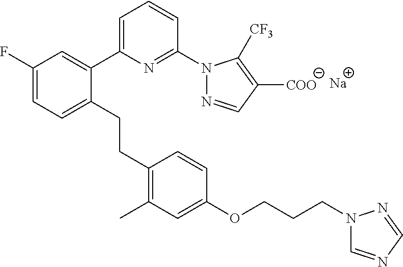

To a solution of previously made 1-(6-(2-(4-(3-(1H-1,2,4-triazol-1-yl)propoxy)-2-methylphenethyl)-5-fluorophenyl)pyridin-2-yl)-5-(trifluoromethyl)-1H-pyrazole-4-carboxylic acid (110 mg, 0.185 mmol) in water (5 mL) and was added sodium hydroxide (7.40 mg, 0.185 mmol) in water (5 mL). The reaction mixture was stirred at RT for 20 min. Then the solution was transferred to lyophilization flask and lyophilized for 20 hours to afford 1-(6-(2-(4-(3-(1H-1,2,4-triazol-1-yl)propoxy)-2-methylphenethyl)-5-fluorophenyl)pyridin-2-yl)-5-(trifluoromethyl)-1H-pyrazole-4-carboxylic acid, sodium salt (93.8 mg, 0.145 mmol, 78% yield) as off-white solid (hygroscopic product). LCMS: rt=2.79 min, m/z=595.2 [M+H]⁺

¹H NMR (d⁶-DMSO), δ (ppm): 8.52 (s, 1H), 8.1 (t, 1H), 7.95 (s, 1H), 7.73 (d, 1H), 7.69 (dd, 1H), 7.54 (dd, 1H), 7.4 (m, 1H), 7.22 (m, 2H), 6.66 (d, 1H), 6.57 (d, 1H), 6.52 (dd, 1H), 4.32 (t, 2H), 3.87 (t, 2H), 2.83 (m, 2H), 2.55 (m, 2H), 2.18 (m, 2H), 1.91 (s, 3H).

### Example 18

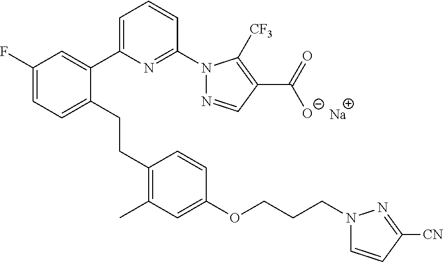 [SMILES: Cc1cc(OCCCn2ccc(C#N)n2)ccc1CCc1ccc(F)cc1-c1cccc(-n2ncc(C(=O)[O-])c2C(F)(F)F)n1 ; [Na+]]

### 1-(6-(2-(4-(3-(3-cyano-1H-pyrazol-1-yl)propoxy)-2-methylphenethyl)-5-fluorophenyl)pyridin-2-yl)-5-(trifluoromethyl)-1H-pyrazole-4-carboxylic acid, sodium salt

### Intermediate 37: Ethyl 1-(6-(2-(4-(3-(3-cyano-1H-pyrazol-1-yl)propoxy)-2-methylphenethyl)-5-fluorophenyl)pyridin-2-yl)-5-(trifluoromethyl)-1H-pyrazole-4-carboxylate

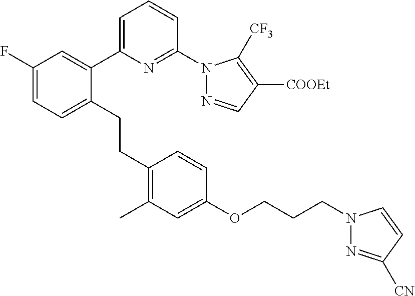 [SMILES: CCOC(=O)c1cnn(-c2cccc(-c3cc(F)ccc3CCc3ccc(OCCCn4ccc(C#N)n4)cc3C)n2)c1C(F)(F)F]

To a solution of ethyl 1-(6-(2-(4-(3-bromopropoxy)-2-methylphenethyl)-5-fluorophenyl)pyridin-2-yl)-5-(trifluoromethyl)-1H-pyrazole-4-carboxylate (400 mg, 0.630 mmol) and 1H-pyrazole-3-carbonitrile (70.4 mg, 0.757 mmol, Fluorochem Products) in acetonitrile (25 mL) was added cesium carbonate (308 mg, 0.946 mmol). The reaction mixture was stirred at 80° C. for 16 hours. The mixture was concentrated under reduced pressure and EtOAc (50 mL) was added. The mixture was filtered through a celite pad and rinsed with EtOAc (2×25 mL). Combined filtrate was concentrated under reduced pressure to afford crude product which was purified by column chromatography eluting with 12-14% EtOAc in hexane to afford ethyl 1-(6-(2-(4-(3-(3-cyano-1H-pyrazol-1-yl)propoxy)-2-methylphenethyl)-5-fluorophenyl)pyridin-2-yl)-5-(trifluoromethyl)-1H-pyrazole-4-carboxylate (180 mg, 0.274 mmol, 43.5% yield) as a gummy product. LCMS: rt=4.28 min, m/z=647.41 [M+H]⁺

### 1-(6-(2-(4-(3-(3-cyano-1H-pyrazol-1-yl)propoxy)-2-methylphenethyl)-5-fluorophenyl)pyridin-2-yl)-5-(trifluoromethyl)-1H-pyrazole-4-carboxylic acid

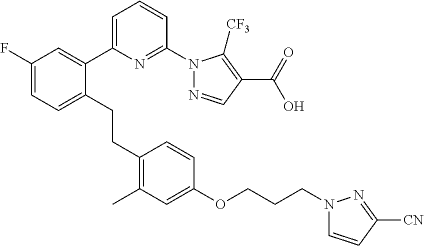 [SMILES: Cc1cc(OCCCn2ccc(C#N)n2)ccc1CCc1ccc(F)cc1-c1cccc(-n2ncc(C(=O)O)c2C(F)(F)F)n1]

To a solution of ethyl 1-(6-(2-(4-(3-(3-cyano-1H-pyrazol-1-yl)propoxy)-2-methylphenethyl)-5-fluorophenyl)pyridin-2-yl)-5-(trifluoromethyl)-1H-pyrazole-4-carboxylate (180 mg, 0.278 mmol) in ethanol (15 mL) was added sodium hydroxide (22.27 mg, 0.557 mmol) in water (1 mL). The reaction mixture was stirred at RT for 16 hours. The solvent was removed by concentration, and the crude was dissolved in water (2 mL) and acidified with a saturated citric acid solution up to pH 4. Product was extracted with EtOAc (3×15 mL) and the combined organic phase was dried over anhydrous Na₂SO₄, filtered and concentrated under reduced pressure. The crude product was purified by Prep-HPLC (method A). Fractions were collected and solvent was removed by concentration. The crude was acidified with diluted acetic acid (pH 4). Product was extracted with EtOAc (3×15 mL), and combined organic phase was dried over anhydrous Na₂SO₄, filtered and concentrated under reduced pressure to afford 1-(6-(2-(4-(3-(3-cyano-1H-pyrazol-1-yl)propoxy)-2-methylphenethyl)-5-fluorophenyl)pyridin-2-yl)-5-(trifluoromethyl)-1H-pyrazole-4-carboxylic acid (78 mg, 0.126 mmol, 45.2% yield). LC/MS: rt=3.29 min, m/z=619.32 [M+H]⁺

¹H NMR (CDCl₃) δ (ppm): 8.15 (s, 1H), 7.95 (t, 1H), 7.55 (d, 1H), 7.4 (m, 2H), 7.25 (m, 1H), 7.05 (m, 2H), 6.65 (d, 1H), 6.6 (d, 1H), 6.55 (d, 1H), 6.45 (m, 1H), 4.35 (t, 2H), 3.80 (t, 2H), 2.95 (m, 2H), 2.65 (m, 2H), 2.3 (m, 2H), 2 (s, 3H).

### Final compound 1-(6-(2-(4-(3-(3-cyano-1H-pyrazol-1-yl)propoxy)-2-methylphenethyl)-5-fluorophenyl)pyridin-2-yl)-5-(trifluoromethyl)-1H-pyrazole-4-carboxylic acid, sodium salt

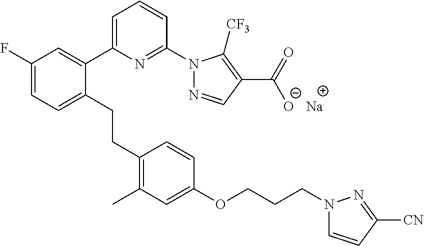 [SMILES: Cc1cc(OCCCn2ccc(C#N)n2)ccc1CCc1ccc(F)cc1-c1cccc(-n2ncc(C(=O)[O-])c2C(F)(F)F)n1 ; [Na+]]

To a solution of the previously made compound, 1-(6-(2-(4-(3-(3-cyano-1H-pyrazol-1-yl)propoxy)-2-methylphenethyl)-5-fluorophenyl)pyridin-2-yl)-5-(trifluoromethyl)-1H-pyrazole-4-carboxylic acid (78 mg, 0.126 mmol) in water (5 mL) was added sodium bicarbonate (10.59 mg, 0.126 mmol) in water (5 mL). The reaction mixture was stirred at RT for 20 min. Then the solution was transferred to a lyophilization flask and lyophilized for 20 hours to afford 1-(6-(2-(4-(3-(3-cyano-1H-pyrazol-1-yl)propoxy)-2-methylphenethyl)-5-fluorophenyl)pyridin-2-yl)-5-(trifluoromethyl)-1H-pyrazole-4-carboxylic acid, sodium salt (53.7 mg, 0.083 mmol, 66.0% yield) as an off white solid (hygroscopic product). LCMS: rt=2.84 min, m/z=619.34 [M+H]⁺.

¹H NMR (d⁶-DMSO) δ (ppm): 8.11 (t, 1H), 8.05 (d, 1H), 7.78 (m, 1H), 7.7 (dd, 1H), 7.55 (dd, 1H), 7.41 (m, 1H), 7.21 (m, 2H), 6.95 (d, 1H), 6.65 (d, 1H), 6.57 (d, 1H), 6.51 (dd, 1H), 4.37 (t, 2H), 3.85 (t, 2H), 2.83 (m, 2H), 2.55 (m, 2H), 2.2 (m, 2H), 1.92 (s, 3H).

### Example 19

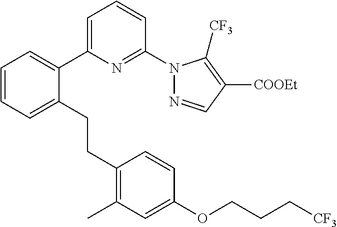 [SMILES: CCOC(=O)c1cnn(-c2cccc(-c3ccccc3CCc3ccc(OCCCC(F)(F)F)cc3C)n2)c1C(F)(F)F]

### Ethyl 1-(6-(2-(2-methyl-4-(4,4,4-trifluorobutoxy)phenethyl)phenyl)pyridin-2-yl)-5-(trifluoromethyl)-1H-pyrazole-4-carboxylate

A mixture of ethyl 1-(6-(2-(4-hydroxy-2-methylphenethyl)phenyl)pyridin-2-yl)-5-(trifluoromethyl)-1H-pyrazole-4-carboxylate (see Intermediate 6) (1 g, 2.018 mmol), potassium carbonate (0.558 g, 4.04 mmol) and 4-bromo-1,1,1-trifluorobutane (0.771 g, 4.04 mmol, Aldrich) in N,N-dimethylformamide (4 mL) was stirred under nitrogen at 100° C. for 16 hours. The reaction mixture was cooled to RT, filtered through a celite pad and rinsed with EtOAc (3×50 mL). Combined filtrate was concentrated under reduced pressure to give the crude product (1.5 g). Crude was first purified by column chromatography eluting with 15-18% EtOAc in hexane to afford a light yellow gum (700 mg) and in a second time was purified by Prep-H PLC using method A. Fractions were collected and lyophilized to afford ethyl 1-(6-(2-(2-methyl-4-(4,4,4-trifluorobutoxy)phenethyl)phenyl)pyridin-2-yl)-5-(trifluoromethyl)-1H-pyrazole-4-carboxylate (260 mg, 0.429 mmol, 21.25% yield) as a white solid. LCMS: rt=4.49 min, m/z=606.22 [M+H]⁺

¹H NMR (CDCl₃), δ (ppm): 8.3 (s, 1H), 7.93 (t, 1H), 7.56 (d, 1H), 7.42 (d, 1H), 7.37 (m, 2H), 7.3 (m, 2H), 6.72 (d, 1H), 6.59 (d, 1H), 6.53 (dd, 1H), 4.38 (q, 2H), 3.96 (t, 2H), 2.93 (m, 2H), 2.7 (m, 2H), 2.29 (m, 2H), 2.02 (m, 5H), 1.37 (t, 3H).

### Example 20

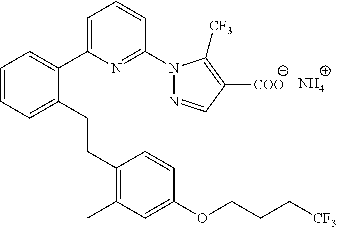

### 1-(6-(2-(2-Methyl-4-(4,4,4-trifluorobutoxy)phenethyl)phenyl)pyridin-2-yl)-5-(trifluoromethyl)-1H-pyrazole-4-carboxylic acid, ammonia salt

To a solution of ethyl 1-(6-(2-(2-methyl-4-(4,4,4-trifluorobutoxy)phenethyl)phenyl)pyridin-2-yl)-5-(trifluoromethyl)-1H-pyrazole-4-carboxylate (2 g, 3.30 mmol) in ethanol (5 mL) and water (0.5 mL) was added sodium hydroxide (0.396 g, 9.91 mmol) at 0° C. and the reaction mixture was allowed to stir at RT for 16 hours. The reaction mixture was concentrated under reduced pressure. The crude was dissolved in cold water (10 mL) and acidified with a saturated citric acid solution up to pH 4. Product was extracted with dichloromethane (3×20 mL). Combined organic phase was washed with water (2×15 mL), dried over anhydrous Na₂SO₄, filtered and concentrated under reduced pressure to give crude product (1.6 g). Purification of 1 g was by Prep-HPLC using method D. Fractions were collected and lyophilized to afford 1-(6-(2-(2-methyl-4-(4,4,4-trifluorobutoxy)phenethyl)phenyl)pyridin-2-yl)-5-(trifluoromethyl)-1H-pyrazole-4-carboxylic acid, ammonia salt (600 mg, 1.023 mmol, 31% yield) as an off white solid. LCMS: rt=3.05 min, m/z=578.28 [M+H]⁺

¹H NMR (d⁶-DMSO) δ (ppm): 8.12 (t, 1H), 7.95 (s, 1H), 7.7 (d, 1H), 7.57 (d, 1H), 7.38 (m, 4H), 6.68 (d, 1H), 6.61 (dd, 1H), 6.54 (dd, 1H), 3.94 (t, 2H), 2.83 (m, 2H), 2.57 (m, 2H), 2.38 (m, 2H), 1.93 (s, 3H), 1.88 (m, 2H).

### Example 21

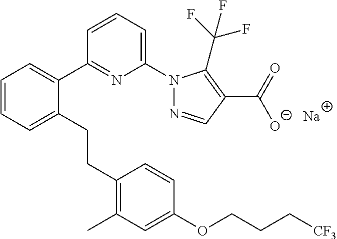 [SMILES: Cc1cc(OCCCC(F)(F)F)ccc1CCc1ccccc1-c1cccc(-n2ncc(C(=O)[O-])c2C(F)(F)F)n1 ; [Na+]]

### 1-(6-(2-(2-methyl-4-(4,4,4-trifluorobutoxy)phenethyl)phenyl)pyridin-2-yl)-5-(trifluoromethyl)-1H-pyrazole-4-carboxylic acid, sodium salt

To a solution of 1-(6-(2-(2-methyl-4-(4,4,4-trifluorobutoxy)phenethyl)phenyl)pyridin-2-yl)-5-(trifluoromethyl)-1H-pyrazole-4-carboxylic acid made according to Example 1 (200 mg, 0.346 mmol) in water (10 mL) was added sodium hydroxide (13.85 mg, 0.346 mmol) at 0° C. and the reaction mixture was allowed to stir at RT for 1 hour. The reaction mixture was lyophilized to afford 1-(6-(2-(2-methyl-4-(4,4,4-trifluorobutoxy)phenethyl)phenyl)pyridin-2-yl)-5-(trifluoromethyl)-1H-pyrazole-4-carboxylic acid, sodium salt (150 mg, 0.250 mmol, 72.1% yield) as an off white solid. LCMS: rt=3.05 min, m/z=578.28 [M+H]⁺

¹H NMR (d⁶-DMSO) δ (ppm): 8.11 (t, 1H), 7.87 (s, 1H), 7.68 (d, 1H), 7.56 (d, 1H), 7.38 (m, 4H), 6.69 (d, 1H), 6.61 (dd, 1H), 6.54 (dd, 1H), 3.94 (t, 2H), 2.84 (m, 2H), 2.58 (m, 2H), 2.38 (m, 2H), 1.94 (s, 3H), 1.88 (m, 2H).

### Example 22

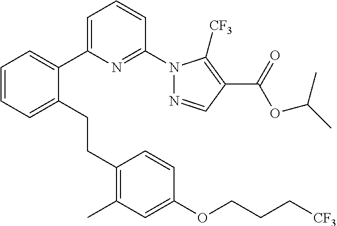 [SMILES: Cc1cc(OCCCC(F)(F)F)ccc1CCc1ccccc1-c1cccc(-n2ncc(C(=O)OC(C)C)c2C(F)(F)F)n1]

### Isopropyl 1-(6-(2-(2-methyl-4-(4,4,4-trifluorobutoxy)phenethyl)phenyl)pyridin-2-yl)-5-(trifluoromethyl)-1H-pyrazole-4-carboxylate

A solution of 1-(6-(2-(2-methyl-4-(4,4,4-trifluorobutoxy)phenethyl)phenyl)pyridin-2-yl)-5-(trifluoromethyl)-1H-pyrazole-4-carboxylic acid made according to Example 1 (450 mg, 0.779 mmol) in thionyl chloride (0.730 mL, 10 mmol) was heated at 75° C. for 1 hour. Then volatile was removed by concentration in vacuo. The crude was cooled to 0° C. and isopropanol (5 mL) was added. The reaction was stirred for 15 min. The reaction mixture was concentrated and diluted with EtOAc (15 mL) and washed with a saturated NaHCO₃ solution (2×10 mL). Organic phase was dried over anhydrous Na₂SO₄, filtered and concentrated under reduced pressure. The crude was purified by Prep-HPLC using method C conditions. Fractions were collected and lyophilized to afford isopropyl 14642-(2-methyl-4-(4,4,4-trifluorobutoxy)phenethyl)phenyl)pyridin-2-yl)-5-(trifluoromethyl)-1H-pyrazole-4-carboxylate (110 mg, 0.171 mmol, 21.99% yield) as a brown gum. LCMS: rt=3.89 min, m/z=620.33 [M+H]⁺

¹H NMR (d⁶-DMSO) δ (ppm): 8.35 (s, 1H), 8.2 (t, 1H), 7.79 (d, 1H), 7.68 (d, 1H), 7.38 (m, 4H), 6.64 (d, 1H), 6.62 (d, 1H), 6.53 (dd, 1H), 5.13 (m, 1H), 3.94 (t, 2H), 2.82 (m, 2H), 2.59 (m, 2H), 2.38 (m, 2H), 1.95 (s, 3H), 1.88 (m, 2H), 1.30 (d, 6H).

### Example 23

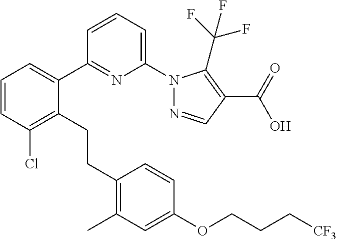 [SMILES: Cc1cc(OCCCC(F)(F)F)ccc1CCc1c(Cl)cccc1-c1cccc(-n2ncc(C(=O)O)c2C(F)(F)F)n1]

### 1-(6-(3-Chloro-2-(2-methyl-4-(4,4,4-trifluorobutoxy)phenethyl)phenyl)pyridin-2-yl)-5-(trifluoromethyl)-1H-pyrazole-4-carboxylic acid

### Intermediate 38: Ethyl 1-(6-(3-chloro-2-formylphenyl)pyridin-2-yl)-5-(trifluoromethyl)-1H-pyrazole-4-carboxylate

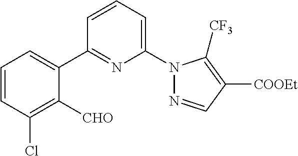 [SMILES: CCOC(=O)c1cnn(-c2cccc(-c3cccc(Cl)c3C=O)n2)c1C(F)(F)F]

To a solution of ethyl 1-(6-chloropyridin-2-yl)-5-(trifluoromethyl)-1H-pyrazole-4-carboxylate (10 g, 31.3 mmol), (3-chloro-2-formylphenyl)boronic acid (8.65 g, 46.9 mmol, Chemblocks) in 1,2-dimethoxyethane (15 mL) and water (2 mL) was added sodium carbonate (6.63 g, 62.6 mmol) and the reaction was stirred under nitrogen at RT. The reaction mixture was purged with argon for 30 min and tetrakis(triphenylphosphine)palladium (3.61 g, 3.13 mmol) was added. The reaction was heated at 110° C. for 16 hours. The reaction mixture was filtered under a celite bed then the filtrate was diluted with water (30 mL) and extracted with EtOAc (3×20 mL), and washed with brine solution (25 mL). The organic layer was separated, dried over anhydrous Na₂SO₄ and concentrated under reduced pressure to afford the crude compound which was purified by column chromatography eluting with 20% EtOAc in hexane. The collected fractions were concentrated under reduced pressure to afford ethyl 1-(6-(3-chloro-2-formylphenyl)pyridin-2-yl)-5-(trifluoromethyl)-1H-pyrazole-4-carboxylate (6 g, 13.15 mmol, 42.0% yield) as off white solid. LCMS: rt=3.21 min, m/z=423.9 [M+H]⁺.

### Intermediate 39: (4-Methoxy-2-methylbenzyl)triphenylphosphonium bromide

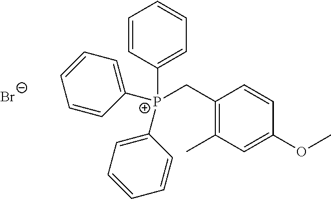 [SMILES: [Br-] ; COc1ccc(C[P+](c2ccccc2)(c2ccccc2)c2ccccc2)c(C)c1]

Under nitrogen, a solution of 1-(bromomethyl)-4-methoxy-2-methylbenzene (8 g, 37.2 mmol) and triphenylphosphine (9.76 g, 37.2 mmol) in toluene (50 mL) was stirred at 100° C. for 16 hours. The reaction mixture was filtered and the solid was washed with toluene (50 mL) to afford (4-methoxy-2-methylbenzyl)triphenylphosphonium bromide (12 g, 24.17 mmol, 65% yield) as off white solid. LCMS: rt=2.39 min, m/z=397.2 (mass-bromine).

### Intermediate 40: (E)-Ethyl 1-(6-(3-chloro-2-(4-methoxy-2-methylstyryl)phenyl)pyridin-2-yl)-5-(trifluoromethyl)-1H-pyrazole-4-carboxylate

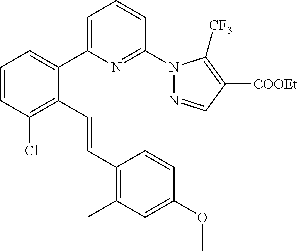 [SMILES: CCOC(=O)c1cnn(-c2cccc(-c3cccc(Cl)c3/C=C/c3ccc(OC)cc3C)n2)c1C(F)(F)F]

To a solution of potassium tert-butoxide (0.662 g, 5.90 mmol) in tetrahydrofuran (10 mL) stirred under nitrogen at 0° C. was added a solution of (4-methoxy-2-methylbenzyl)triphenylphosphonium bromide (2.253 g, 4.72 mmol) in tetrahydrofuran (10 mL) portion-wise during 5 min and the reaction was stirred for 10 min. Then ethyl 1-(6-(3-chloro-2-formylphenyl)pyridin-2-yl)-5-(trifluoromethyl)-1H-pyrazole-4-carboxylate (1 g, 2.360 mmol) was added. The reaction mixture was stirred at RT for 16 hours. The reaction mixture was quenched with saturated ammonium chloride solution (10 mL) and extracted with EtOAc (3×20 mL), washed with brine solution (25 mL). The organic layer was separated, dried over anhydrous Na₂SO₄ and concentrated under reduced pressure to afford the crude compound which was purified by column chromatography eluting with 20% EtOAc in hexane. The collected fractions were concentrated under reduced pressure to afford (E)-ethyl 1-(6-(3-chloro-2-(4-methoxy-2-methylstyryl)phenyl)pyridin-2-yl)-5-(trifluoromethyl)-1H-pyrazole-4-carboxylate (850 mg, 1.031 mmol, 43.7% yield) as a off white solid. LCMS: rt=3.31 min, m/z=542.24 [M+H]⁺

### Intermediate 41: Ethyl 1-(6-(3-chloro-2-(4-methoxy-2-methylphenethyl)phenyl)pyridin-2-yl)-5-(trifluoromethyl)-1H-pyrazole-4-carboxylate

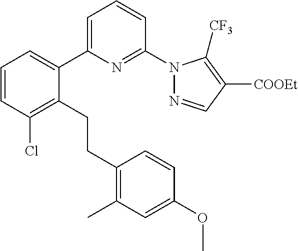 [SMILES: CCOC(=O)c1cnn(-c2cccc(-c3cccc(Cl)c3CCc3ccc(OC)cc3C)n2)c1C(F)(F)F]

To a solution of (E)-ethyl 1-(6-(3-chloro-2-(4-methoxy-2-methylstyryl)phenyl)pyridin-2-yl)-5-(trifluoromethyl)-1H-pyrazole-4-carboxylate (650 mg, 1.199 mmol) in methanol (25 mL) was added Pd/C (128 mg, 0.120 mmol). The reaction mixture was stirred at RT under hydrogen atmosphere (15 psi) for 2 hours. The reaction mixture was filtered on a celite bed and washed with methanol (20 mL), then the filtrate was concentrated to afford ethyl 1-(6-(3-chloro-2-(4-methoxy-2-methylphenethyl)phenyl)pyridin-2-yl)-5-(trifluoromethyl)-1H-pyrazole-4-carboxylate (590 mg, 0.596 mmol, 49.7% yield) as a gummy liquid. LCMS: rt=3.32 min, m/z=544.18 [M+H]⁺

### Intermediate 42: Ethyl 1-(6-(3-chloro-2-(4-hydroxy-2-methylphenethyl)phenyl)pyridin-2-yl)-5(trifluoromethyl)-1H-pyrazole-4-carboxylate

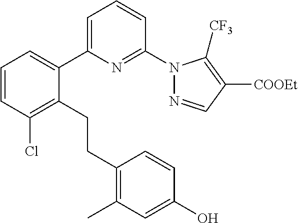 [SMILES: CCOC(=O)c1cnn(-c2cccc(-c3cccc(Cl)c3CCc3ccc(O)cc3C)n2)c1C(F)(F)F]

To a solution of ethyl 1-(6-(3-chloro-2-(4-methoxy-2-methylphenethyl)phenyl)pyridin-2-yl)-5-(trifluoromethyl)-1H-pyrazole-4-carboxylate (590 mg, 1.085 mmol) in dichloromethane (10 mL) under nitrogen at 0° C. was added dropwise boron tribromide (0.103 mL, 1.085 mmol) during 5 min. The reaction mixture was stirred at RT for 2 hours. The reaction mixture was diluted with water (10 mL) and washed with a sodium bicarbonate solution (50 mL) then extracted with EtOAc (3×20 mL), and washed with brine solution (25 mL). The organic layer was separated, dried over anhydrous Na₂SO₄ and concentrated under reduced pressure to afford the crude material which was purified by prep-HPLC using method A conditions. Fractions were collected and concentrated under vacuum. The residue was put into water (25 ml), extracted with EtOAc (3×20 mL), washed with brine solution (25 mL). The organic layer was separated, dried over anhydrous Na₂SO₄ and concentrated under reduced pressure to afford ethyl 1-(6-(3-chloro-2-(4-hydroxy-2-methylphenethyl)phenyl)pyridin-2-yl)-5-(trifluoromethyl)-1H-pyrazole-4-carboxylate (320 mg, 0.595 mmol, 54.8% yield) as a colorless gummy liquid. LCMS: rt=2.99 min, m/z=530.25 [M+H]⁺

### Intermediate 43: Ethyl 1-(6-(3-chloro-2-(2-methyl-4-(4,4,4-trifluorobutoxy)phenethyl)phenyl)pyridin-2-yl)-5-(trifluoromethyl)-1H-pyrazole-4-carboxylate

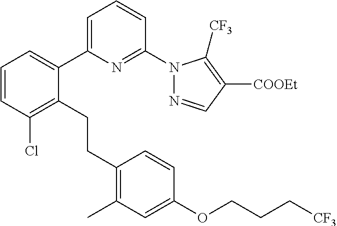 [SMILES: CCOC(=O)c1cnn(-c2cccc(-c3cccc(Cl)c3CCc3ccc(OCCCC(F)(F)F)cc3C)n2)c1C(F)(F)F]

At 0° C., under nitrogen, to a solution of ethyl 1-(6-(3-chloro-2-(4-hydroxy-2-methylphenethyl)phenyl)pyridin-2-yl)-5-(trifluoromethyl)-1H-pyrazole-4-carboxylate (100 mg, 0.189 mmol), and 4-bromo-1,1,1-trifluorobutane (76 mg, 0.396 mmol) in N,N-dimethylformamide (5 mL) was added potassium carbonate (52.2 mg, 0.377 mmol). The reaction mixture was stirred at 100° C. for 16 hours. The reaction was not completed. 0.5 eq of 4-bromo-1,1,1-trifluorobutane (17 mg) was added. The reaction mixture was stirred at 100° C. for another 20 hours. The reaction mixture was diluted with water (10 mL), extracted with EtOAc (3×20 mL), and washed with brine solution (25 mL). The organic layer was separated, dried over anhydrous Na₂SO₄ and concentrated under reduced pressure to afford crude compound which was purified by Prep-HPLC using method A conditions. Fractions were collected and concentrated under reduced pressure. The residue was dissolved into EtOAc (20 mL). The organic layer was separated and dried over anhydrous Na₂SO₄, then concentrated under reduced pressure to get a ethyl 1-(6-(3-chloro-2-(2-methyl-4-(4,4,4-trifluorobutoxy)phenethyl)phenyl)pyridin-2-yl)-5-(trifluoromethyl)-1H-pyrazole-4-carboxylate (35 mg, 0.052 mmol, 27.5% yield) as colorless liquid. LCMS: rt=3.50 min, m/z=640.36 [M+H]⁺

### Example 23

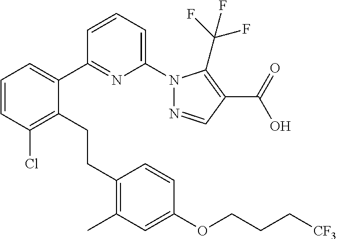 [SMILES: Cc1cc(OCCCC(F)(F)F)ccc1CCc1c(Cl)cccc1-c1cccc(-n2ncc(C(=O)O)c2C(F)(F)F)n1]

### 1-(6-(3-Chloro-2-(2-methyl-4-(4,4,4-trifluorobutoxy)phenethyl)phenyl)pyridin-2-yl)-5-(trifluoromethyl)-1H-pyrazole-4-carboxylic acid

To a solution of ethyl 1-(6-(3-chloro-2-(2-methyl-4-(4,4,4-trifluorobutoxy)phenethyl)phenyl)pyridin-2-yl)-5-(trifluoromethyl)-1H-pyrazole-4-carboxylate (35 mg, 0.055 mmol) in ethanol (3 mL) was added sodium hydroxide (3.28 mg, 0.082 mmol) in water (1 mL). The reaction mixture was stirred at RT for 4 hours. The reaction mixture was concentrated under reduced pressure. The residue was diluted with water (10 mL) and adjusted to pH-5 using acetic acid and extracted with dichloromethane (2×10 mL). Then the combined organic layer was dried over anhydrous sodium sulphate, filtered and concentrated under reduced pressure to afford crude. The crude was washed with n-pentane and dried under reduced pressure to afford 1-(6-(3-chloro-2-(2-methyl-4-(4,4,4-trifluorobutoxy)phenethyl)phenyl)pyridin-2-yl)-5-(trifluoromethyl)-1H-pyrazole-4-carboxylic acid (21 mg, 0.034 mmol, 61.8% yield) as off white gum. LCMS: rt=3.14 min, m/z=610.36 [M+H]⁺

¹H NMR (d⁶-DMSO), δ (ppm): 13.5 (brs, 1H), 8.31 (brs, 1H), 8.21 (t, 1H), 7.84 (d, 1H), 7.63 (d, 1H), 7.6 (dd, 1H), 7.39 (d, 1H), 7.34 (dd, 1H), 6.6 (brs, 1H), 6.5 (m, 2H), 3.94 (t, 2H), 2.86 (m, 2H), 2.63 (m, 2H), 2.35 (m, 2H), 1.92 (s, 3H), 1.88 (m, 2H).

Compounds, for example agents activating sGC as disclosed herein, can be used as a medicament or used to formulate a pharmaceutical composition with one or more of the utilities disclosed herein. They can be administered in vitro to cells in culture, in vivo to cells in the body, or ex vivo to cells outside of an individual that can later be returned to the body of the same individual or another. Such cells can be desegregated or provided as solid tissue.

Compounds, for example agents activating sGC as disclosed herein can be used to produce a medicament or other pharmaceutical compositions. Use of agents activating sGC which further comprise a pharmaceutically acceptable carrier and compositions which further comprise components useful for delivering the composition to an individual are known in the art. Addition of such carriers and other components to the agents as disclosed herein is well within the level of skill in this art. In addition, there are a number of resources that are available to the skilled artisan which describe pharmaceutically acceptable excipients and may be useful in selecting suitable pharmaceutically acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

In addition to the active compound, such compositions can contain pharmaceutically-acceptable carriers and other ingredients known to facilitate administration and/or enhance uptake (e.g., saline, dimethyl sulfoxide, lipid, polymer, affinity-based cell specific-targeting systems). The composition can be incorporated in a gel, sponge, or other permeable matrix (e.g., formed as pellets or a disk) and placed in proximity to the endothelium for sustained, local release. The composition can be administered in a single dose or in multiple doses which are administered at different time intervals.

The compounds of this invention can be administered as topical eye drops. The compounds of this invention can be administered via sub-conjunctival, intracameral or intravitreal routes which would necessitate administration intervals that are longer than daily.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation, for example the carrier does not decrease the impact of the agent on the treatment. In other words, a carrier is pharmaceutically inert.

The pharmaceutical compositions of the invention are prepared using techniques and methods known to those skilled in the art. Some of the methods commonly used in the art are described in Remington's Pharmaceutical Sciences (Mack Publishing Company). Accordingly, another embodiment of this invention is a method of preparing a pharmaceutical composition comprising the step of admixing a compound of Formula (I) with one or more pharmaceutically acceptable excipients.

Treatment of the diseases or disorders described herein can be achieved using a compound of this invention as a monotherapy, or in dual or multiple combination therapy. The compounds of Formula (I) and pharmaceutically acceptable salts thereof may be employed alone or in combination with other therapeutic agents. Combination therapies according to the present invention thus comprise the administration of at least one compound of Formula (I) or a pharmaceutically acceptable salt thereof, and at least one other therapeutically active agent. Preferably, combination therapies according to the present invention comprise the administration of a compound of Formula (I) or a pharmaceutically acceptable salt thereof, and at least one other therapeutically active agent. The compound of Formulas (I) and pharmaceutically acceptable salts thereof, and the other therapeutically active agent(s) may be administered together in a single pharmaceutical composition or separately and, when administered separately this may occur simultaneously or sequentially in any order. The amounts of the compound of Formulas (I) and a pharmaceutically acceptable salt thereof, and the other therapeutically active agent(s) and the relative timings of administration will be selected in order to achieve the desired combined therapeutic effect.

In the context of this invention, combination therapies would include other IOP-lowering drugs, for example prostaglandin analogs (e.g., latanoprost, bimatoprost, travoprost, tafluprost); beta-adrenergic blockers (e.g., timolol, betaxolol, levobunolol); alpha-adrenergic agonists (e.g., brimonidine, paraamino-clonidine); parasympathomimetics (e.g. pilocarpine, carbachol, acethylcholineesterase inhibitors); sympathomimetics (e.g., epinephrine, dipivalyl-epinephrine); and carbonic anhydrase inhibitors (e.g., dorzolamide, brinzolamide). In one embodiment, a compound of this invention is administered in combination with a prostaglandin analog (e.g., latanoprost, bimatoprost, travoprost, or tafluprost). In another embodiment, a compound of this invention is administered in combination with a beta-adrenergic blocker (e.g., timolol, betaxolol, levobunolol). In yet another embodiment, a compound of this invention is administered in combination with an alpha-adrenergic agonist (e.g., brimonidine, paraamino-clonidine). In still yet another embodiment, a compound of this invention is administered in combination with a carbonic anhydrase inhibitor (e.g., dorzolamide, brinzolamide).

Pharmaceutical formulations adapted for topical administration may be formulated as ointments, creams, emulsions, suspensions, lotions, powders, solutions, pastes, gels, sprays, aerosols or oils.

For treatments of the eye or other external tissues, for example mouth and skin, the formulations may be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent. Formulations to be administered to the eye will have ophthalmically compatible pH and osmolality. One or more ophthalmically acceptable pH adjusting agents and/or buffering agents can be included in a composition of the invention, including acids such as acetic, boric, citric, lactic, phosphoric and hydrochloric acids; bases such as sodium hydroxide, sodium phosphate, sodium borate, sodium citrate, sodium acetate, and sodium lactate; and buffers such as citrate/dextrose, sodium bicarbonate and ammonium chloride. Such acids, bases, and buffers can be included in an amount required to maintain pH of the composition in an ophthalmically acceptable range. One or more ophthalmically acceptable salts can be included in the composition in an amount sufficient to bring osmolality of the composition into an ophthalmically acceptable range. Such salts include those having sodium, potassium or ammonium cations and chloride, citrate, ascorbate, borate, phosphate, bicarbonate, sulfate, thiosulfate or bisulfite anions.

The ocular delivery device may be designed for the controlled release of one or more therapeutic agents with multiple defined release rates and sustained dose kinetics and permeability. Controlled release may be obtained through the design of polymeric matrices incorporating different choices and properties of biodegradable/bioerodable polymers (e.g. poly(ethylene vinyl) acetate (EVA), superhydrolyzed PVA), hydroxyalkyl cellulose (HPC), methylcellulose (MC), hydroxypropyl methyl cellulose (HPMC), polycaprolactone, poly(glycolic) acid, poly(lactic) acid, polyanhydride, of polymer molecular weights, polymer crystallinity, copolymer ratios, processing conditions, surface finish, geometry, excipient addition and polymeric coatings that will enhance drug diffusion, erosion, dissolution and osmosis.

Formulations for drug delivery using ocular devices may combine one or more active agents and adjuvants appropriate for the indicated route of administration. For example, the active agents may be admixed with any pharmaceutically acceptable excipient, lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, tableted or encapsulated for conventional administration. Alternatively, the compounds may be dissolved in polyethylene glycol, propylene glycol, carboxymethyl cellulose colloidal solutions, ethanol, corn oil, peanut oil, cottonseed oil, sesame oil, tragacanth gum, and/or various buffers. The compounds may also be mixed with compositions of both biodegradable and non-biodegradable polymers, and a carrier or diluent that has a time delay property. Representative examples of biodegradable compositions can include albumin, gelatin, starch, cellulose, dextrans, polysaccharides, poly (D,L-lactide), poly (D,L-lactide-co-glycolide), poly (glycolide), poly (hydroxybutyrate), poly (alkylcarbonate) and poly (orthoesters) and mixtures thereof. Representative examples of non-biodegradable polymers can include EVA copolymers, silicone rubber and poly (methylacrylate), and mixtures thereof.

Pharmaceutical compositions for ocular delivery also include in situ gellable aqueous composition. Such a composition comprises a gelling agent in a concentration effective to promote gelling upon contact with the eye or with lacrimal fluid. Suitable gelling agents include but are not limited to thermosetting polymers. The term “in situ gellable” as used herein includes not only liquids of low viscosity that form gels upon contact with the eye or with lacrimal fluid, but also includes more viscous liquids such as semi-fluid and thixotropic gels that exhibit substantially increased viscosity or gel stiffness upon administration to the eye. See, for example, Ludwig (2005) Adv. Drug Deliv. Rev. 3; 57:1595-639, herein incorporated by reference for purposes of its teachings of examples of polymers for use in ocular drug delivery.

### Biological Examples

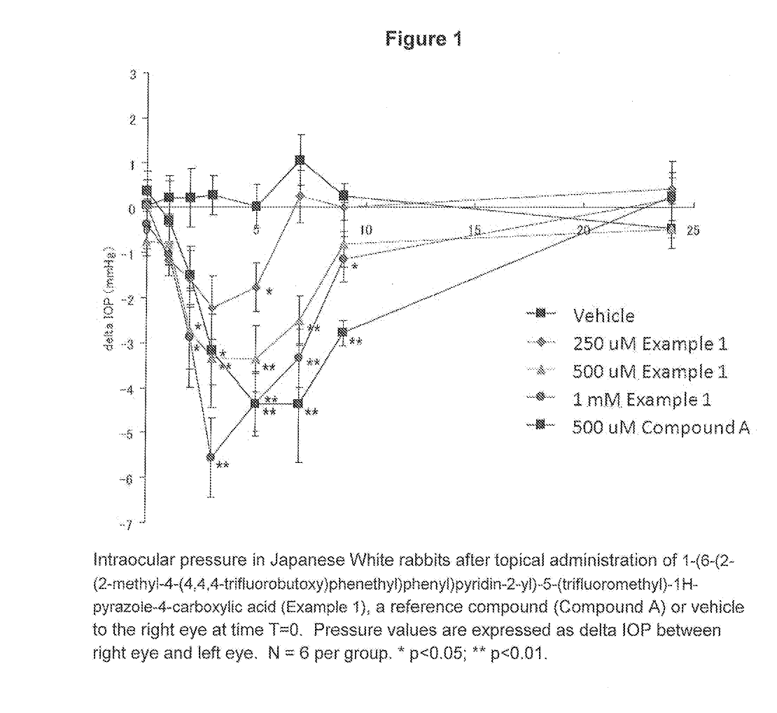

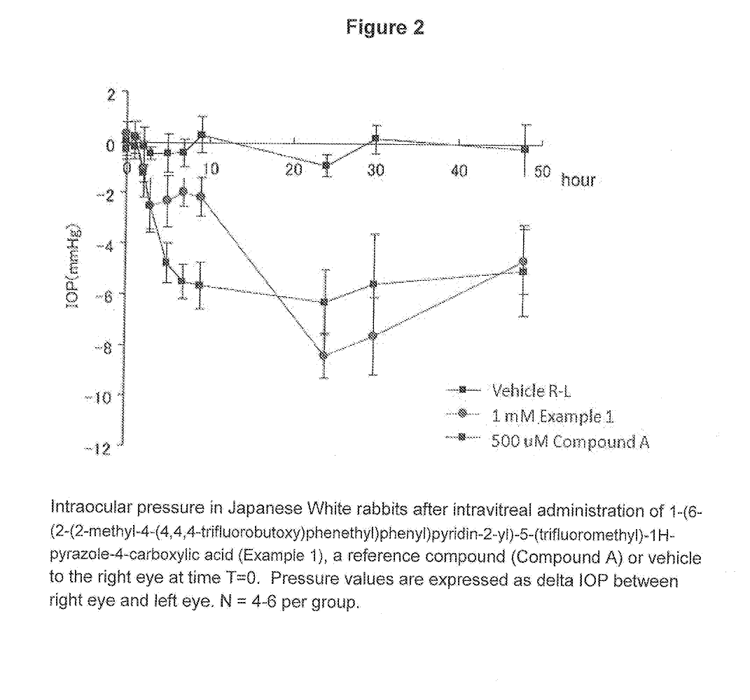

The present invention is demonstrated with in vivo data. In Japanese White rabbits, IOP was measured at baseline (immediately preceding administration of test article) and at predetermined time points (1, 2, 3, 5, 7, 9, 24 hours; additional time points were 30 and 48 hours after intravitreal administration) after topical (FIG. 1) or intravitreal (FIG. 2) administration of ophthalmic formulations containing drug, vehicle or saline using applanation tonometry. Test articles were topically administered in a 50 microliter volume to the right eye, saline in a 50 microliter volume to the contralateral, left eye. For intravitreal administration, dosing volumes were 20 microliters instead. For each animal, the difference between right eye IOP and left eye IOP was calculated as delta IOP. Following topical administration, 1-(6-(2-(2-methyl-4-(4,4,4-trifluorobutoxy)phenethyl)phenyl)pyridin-2-yl)-5-(trifluoromethyl)-1H-pyrazole-4-carboxylic acid dose-dependently and effectively decreased IOP as shown in FIG. 1. After intravitreal administration 1-(6-(2-(2-methyl-4-(4,4,4-trifluorobutoxy)phenethyl)phenyl)pyridin-2-yl)-5-(trifluoromethyl)-1H-pyrazole-4-carboxylic acid produced a long-lasting and effective reduction in IOP for at least 48 hours as depicted in FIG. 2.

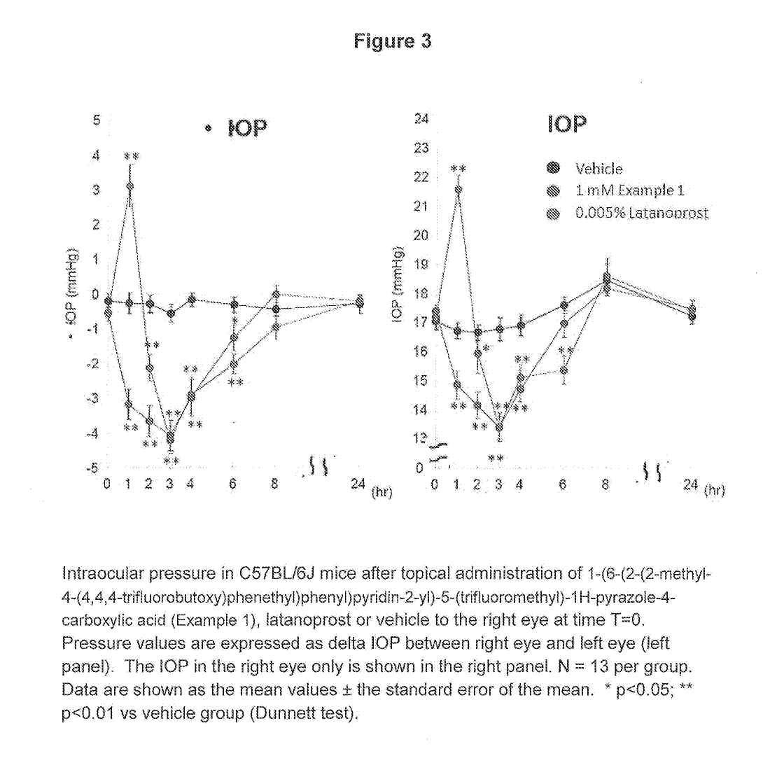

Additionally, the effect on IOP in normal mice was also assessed. In C57BL/6J mice, IOP was measured at baseline (immediately preceding administration of test article) and at predetermined time points (1, 2, 3, 4, 6, 8, 24 hours) after topical administration of ophthalmic formulations containing drug, vehicle or saline using a TonoLab. Test articles were administered in a 4 microliter volume to the right eye, saline in a 4 microliter volume to the contralateral, left eye. For each animal, the difference between right eye IOP and left eye IOP was calculated as delta IOP. As depicted in FIG. 3 below, 1-(6-(2-(2-methyl-4-(4,4,4-trifluorobutoxy)phenethyl)phenyl)pyridin-2-yl)-5-(trifluoromethyl)-1H-pyrazole-4-carboxylic acid lowered IOP with efficacy similar to latanoprost, but without the initial hypertensive spike.

Biological Enzyme Assay

The activity of soluble guanylate cyclase (sGC) was tested in an assay based on measuring the fluorescent polarisation (FP) signal of fluorescently labelled cGMP. FP increased on interaction with an anti-cGMP antibody as the motility of the molecule was reduced. Newly produced cGMP displaced the interaction giving rise to a decrease in polarisation and FP signal which was equated to enzyme activity. Compounds were incubated with human sGC, anti-cGMP antibody, the GTP substrate and fluorescently labelled cGMP. After a period of one hour the assay was stopped with the addition of EDTA and after a further hour the assay was read.

Human sGC was thawed and resuspended in assay buffer (100 mM TRIS, 10 mM MgCl₂, 0.2 mM Tween 20, pH7.4, containing 1:100 dilution of sheep anti-cGMP) to give final concentration of 1 nM in the well. A substrate solution was prepared containing GTP and 8-fluo-cGMP in de-ionized water to a final concentration of 25 μM and 50 nM respectively. Assay plates containing 5 μL of various test compounds and of a standard agonist (50 μM-50 nM) in 1% DMSO as 6 point, four fold dilutions across a 96 well plate were used in the assay. The plate also contained 6 wells of DMSO (1%) to produce high control and a cGMP standard curve (14 nM to 10 μM) to convert FP data to cGMP concentration. 25 μL of enzyme mix and 20 μl of substrate mix described above were added to each well of the plate. Samples were mixed on an orbital shaker and then incubated at room temperature for 1 hour. After this incubation period 5 μl of 0.5M EDTA was added to all wells and the plates were incubated for a further hour at room temperature prior to reading the FP signal in an appropriate reader. For data handling FP data were converted to cGMP concentrations and then fitted using ActivityBase software. The activity of a test compound was determined as the pEC500 value which is the concentration able to increase by 5-fold basal cGMP.

The pEC500 of 1-(6-(2-(2-methyl-4-(4,4,4-trifluorobutoxy)phenethyl)phenyl)pyridin-2-yl)-5-(trifluoromethyl)-1H-pyrazole-4-carboxylic acid was determined as 6.95 in this assay.

pEC500 scores for other compounds that fall within the scope of this invention are found below.

| Example No. | sGC pEC500 |
|---|---|
| 1 | 6.95 |
| 2 | 6.19 |
| 3 | 7.88 |
| 4 | 6.6 |
| 5 | 6.88 |
| 6 | 7.4 |
| 7 | 7.1 |
| 8 | 7.28 |
| 9 | 7.73 |
| 10 | 7.79 |
| 11 | 7.47 |
| 12 | 6.59 |

Biological Cellular Assay

The activity of soluble guanylate cyclase (sGC) was tested in an assay based on measuring phosphorylation of the protein kinase G (PKG) substrate vasodilator-stimulated phosphoprotein (VASP) in rat aortic smooth muscle cells. Primary rat aortic smooth muscle cells were incubated at 37° C. for 10 min in the presence of 10 μM 1H-[1,2,4]oxadiazolo[4,3-a]quinoxalin-1-one (ODQ), a highly selective, and irreversible sGC heme iron oxidant. Dimethylsulfoxide (DMSO) vehicle and varying concentrations of the compound to be tested were then added. Following a 30 min incubation at 37° C., media was aspirated and the cells were rinsed with phosphate-buffered saline (PBS) and fixed with 4% formaldehyde in PBS by incubating at room temperature for 20 min. Cells were then washed with PBS and permeabilized for 10 min using 0.1% triton X-100 in PBS. Following PBS rinsing, the cells were blocked for 90 min at room temperature with blocking buffer. The buffer was aspirated and the cells were treated overnight at 4° C. with primary antibody (pSer239-VASP, rabbit polyclonal Ab) diluted 1:500 in blocking buffer. Following three washes with 0.05% Tween 20, the cells were treated for 1 h at room temperature with a fluorescent labeled secondary antibody (IRDye® 800CW Donkey Anti-Rabbit IgG) diluted 1:2500 in blocking buffer with 0.05% Tween 20. Following two washes with PBS, infrared fluorescence was measured using an Odyssey Infrared Imaging System. The activity of a test compound was determined as the pEC50 value which is the concentration able to increase by 50% (vs. Bmax) the phospho-VASP fluorescent signal. Front. Pharmacol., 5 Jul. 2012|doi: 10.3389/fphar.2012.00128, Volume 3 July, 2012, Article number 128.

pEC50 values for compounds that fall within the scope of this invention are found below.

| Example No. | Cell assay pEC50 |
|---|---|
| 1 | 7.84 |
| 3 | 8.23 |
| 5 | 7.22 |
| 6 | 6.82 |
| 9 | <6.2 |
| 10 | 7.04 |
| 11 | 6.99 |
| 13 | 8.9 |
| 14 | 8.8 |
| 15 | 7.4 |
| 16 | 8.2 |
| 17 | 7.7 |
| 18 | 8.1 |
| 19 | 6.6 |
| 21 | 8.1 |
| 22 | 5.9 |

1. A compound according to formula (I): 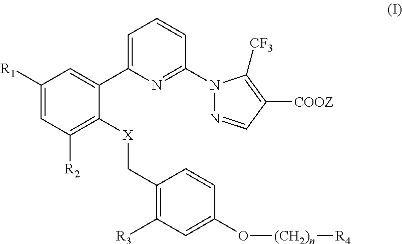
or a pharmaceutically acceptable salt thereof, wherein:
R1 and R2 are each independently selected from H and halogen;
R3 is selected from H, —CH₃ and F;
R4 is selected from —CF₃, —OCH₃, —CN, —COOH, morpholine, 3-(trifluoromethyl)-1-pyrazolyl, an optionally substituted 5- to 6-membered heteroaryl ring, wherein the optional substituents are independently —CN or —OCH₃, and an optionally substituted 5- to 6-membered heterocyclic ring;
X is selected from O and CH₂;
Z is selected from H and C₁₋₄ alkyl; and
n is 2 or 3.

2. The compound or salt according to claim 1, wherein:
R1 and R2 are each H;
R3 is selected from H, —CH₃ and F;
R4 is selected from —CF₃, —OCH₃, —CN, —COOH, morpholine, 3-(trifluoromethyl)-1-pyrazolyl, an optionally substituted 5- to 6-membered heteroaryl ring, wherein the optional substituents are independently —CN or —OCH₃, and an optionally substituted 5- to 6-membered heterocyclic ring;
X is selected from O and CH₂;
Z is selected from H and C₁₋₄ alkyl; and
n is 3.

3. The compound or salt according to claim 1, wherein:
R1 and R2 are each halogen;
R3 is selected from H, —CH₃ and F;
R4 is selected from —CF₃, —OCH₃, —CN, —COOH, morpholine, 3-(trifluoromethyl)-1-pyrazolyl, an optionally substituted 5- to 6-membered heteroaryl ring, wherein the optional substituents are independently —CN or —OCH₃, and an optionally substituted 5- to 6-membered heterocyclic ring;
X is selected from O and CH₂;
Z is selected from H and C₁₋₄ alkyl; and
n is 3.

4. The compound or salt according to claim 2, wherein:
R1 and R2 are each H;
R3 is selected from H and —CH₃;
R4 is selected from —CF₃, —OCH₃, —CN, —COOH, morpholine, 3-(trifluoromethyl)-1-pyrazolyl, an optionally substituted 5- to 6-membered heteroaryl ring, wherein the optional substituents are independently —CN or —OCH₃, and an optionally substituted 5- to 6-membered heterocyclic ring;
X is selected from O and CH₂;
Z is selected from H and C₁₋₄ alkyl; and
n is 3.

5. The compound or salt according to claim 3, wherein:
R1 and R2 are each halogen;
R3 is selected from H, and —CH₃;
R4 is selected from —CF₃, —OCH₃, —CN, —COOH, morpholine, 3-(trifluoromethyl)-1-pyrazolyl, an optionally substituted 5- to 6-membered heteroaryl ring, wherein the optional substituents are independently —CN or —OCH₃, and an optionally substituted 5- to 6-membered heterocyclic ring;
X is selected from O and CH₂;
Z is selected from H and C₁₋₄ alkyl; and
n is 3.

6. The compound or salt according to claim 1, wherein:
R1 and R2 are each independently selected from H and halogen;
R3 is selected from H and —CH₃;
R4 is selected from —CF₃, —OCH₃, —CN, —COOH, morpholine, 3-(trifluoromethyl)-1-pyrazolyl, an optionally substituted 5- to 6-membered heteroaryl ring, wherein the optional substituents are independently —CN or —OCH₃, and an optionally substituted 5- to 6-membered heterocyclic ring;
X is selected from O and CH₂;
Z is selected from H and C₁₋₄ alkyl; and
n is 2 or 3.

7. The compound or salt according to claim 6, wherein:
R1 and R2 are each independently selected from H and halogen;
R3 is selected from H and —CH₃;
R4 is selected from —CF₃, —OCH₃, —CN, —COOH, morpholine, 3-(trifluoromethyl)-1-pyrazolyl, an optionally substituted 5- to 6-membered heteroaryl ring, wherein the optional substituents are independently —CN or —OCH₃, and an optionally substituted 5- to 6-membered heterocyclic ring;
X is CH₂;
Z is selected from H and C₁₋₄ alkyl; and
n is 3.

8. The compound or salt according to claim 7, wherein:
R1 and R2 are each independently selected from H and halogen;
R3 is —CH₃;
R4 is selected from —CF₃, —OCH₃, —CN, —COOH, morpholine, 3-(trifluoromethyl)-1-pyrazolyl, an optionally substituted 5- to 6-membered heteroaryl ring, wherein the optional substituents are independently —CN or —OCH₃, and an optionally substituted 5- to 6-membered heterocyclic ring;
X is CH₂;
Z is selected from H and C₁₋₄ alkyl; and
n is 3.

9. The compound or salt according to claim 7, wherein:
R1 and R2 are each independently selected from H and halogen;
R3 is H;
R4 is selected from —CF₃, —OCH₃, —CN, —COOH, morpholine, 3-(trifluoromethyl)-1-pyrazolyl, an optionally substituted 5- to 6-membered heteroaryl ring, wherein the optional substituents are independently —CN or —OCH₃, and an optionally substituted 5- to 6-membered heterocyclic ring;
X is CH₂;
Z is selected from H and C₁₋₄ alkyl; and
n is 3.

10. The compound or salt according to claim 6, wherein:
R1 and R2 are each independently selected from H and halogen;
R3 is selected from H and —CH₃;
R4 is selected from —CF₃, —OCH₃, —CN, —COOH, morpholine, 3-(trifluoromethyl)-1-pyrazolyl, an optionally substituted 5- to 6-membered heteroaryl ring, wherein the optional substituents are independently —CN or —OCH₃, and an optionally substituted 5- to 6-membered heterocyclic ring;
X is O;
Z is selected from H and C₁₋₄ alkyl; and
n is 3.

11. The compound or salt according to claim 10, wherein:
R1 and R2 are each independently selected from H and halogen;
R3 is —CH₃;
R4 is selected from —CF₃, —OCH₃, —CN, —COOH, morpholine, 3-(trifluoromethyl)-1-pyrazolyl, an optionally substituted 5- to 6-membered heteroaryl ring, wherein the optional substituents are independently —CN or —OCH₃, and an optionally substituted 5- to 6-membered heterocyclic ring;
X is O;
Z is selected from H and C₁₋₄ alkyl; and
n is 3.

12. The compound or salt according to claim 6, wherein:
R1 and R2 are each independently selected from H and halogen;
R3 is H;
R4 is selected from —CF₃, —OCH₃, —CN, —COOH, morpholine, 3-(trifluoromethyl)-1-pyrazolyl, an optionally substituted 5- to 6-membered heteroaryl ring, wherein the optional substituents are independently —CN or —OCH₃, and an optionally substituted 5- to 6-membered heterocyclic ring;
X is O;
Z is selected from H and C₁₋₄ alkyl; and
n is 3.

13. A compound, or a pharmaceutically acceptable salt thereof, which is:
1-(6-(2-(2-methyl-4-(4,4,4-trifluorobutoxy)phenethyl)phenyl)pyridin-2-yl)-5-(trifluoromethyl)-1H-pyrazole-4-carboxylic acid;
1-(6-(2-(4-(3-morpholinopropoxy)phenethyl)phenyl)pyridin-2-yl)-5-(trifluoromethyl)-1H-pyrazole-4-carboxylic acid;
1-(6-(2-(2-methyl-4-(3-(3-(trifluoromethyl)-1H-pyrazol-1-yl)propoxy)phenethyl)phenyl)pyridin-2-yl)-5-(trifluoromethyl)-1H-pyrazole-4-carboxylic acid;
1-(6-(2-((4-(2-methoxyethoxy)benzyl)oxy)phenyl)pyridin-2-yl)-5-(trifluoromethyl)-1H-pyrazole-4-carboxylic acid;
1-(6-(2-((2-fluoro-4-(4,4,4-trifluorobutoxy)benzyl)oxy)phenyl)pyridin-2-yl)-5-(trifluoromethyl)-1H-pyrazole-4-carboxylic acid;
1-(6-(2-((2-fluoro-4-(3-methoxypropoxy)benzyl)oxy)phenyl)pyridin-2-yl)-5-(trifluoromethyl)-1H-pyrazole-4-carboxylic acid;
1-(6-(2-((4-(3-methoxypropoxy)benzyl)oxy)phenyl)pyridin-2-yl)-5-(trifluoromethyl)-1H-pyrazole-4-carboxylic acid;
1-(6-(2-((4-(3-cyanopropoxy)benzyl)oxy)phenyl)pyridin-2-yl)-5-(trifluoromethyl)-1H-pyrazole-4-carboxylic acid;
1-(6-(2-((4-(3-cyanopropoxy)-2-methylbenzyl)oxy)phenyl)pyridin-2-yl)-5-(trifluoromethyl)-1H-pyrazole-4-carboxylic acid;
1-(6-(2-((4-(3-methoxypropoxy)-2-methylbenzyl)oxy)phenyl)pyridin-2-yl)-5-(trifluoromethyl)-1H-pyrazole-4-carboxylic acid;
1-(6-(2-((2-methyl-4-(4,4,4-trifluorobutoxy)benzyl)oxy)phenyl)pyridin-2-yl)-5-(trifluoromethyl)-1H-pyrazole-4-carboxylic acid;
1-(6-(2-((4-(3-carboxypropoxy)-2-methylbenzyl)oxy)phenyl)pyridin-2-yl)-5-(trifluoromethyl)-1H-pyrazole-4-carboxylic acid;
1-(6-(5-fluoro-2-(2-methyl-4-(4,4,4-trifluorobutoxy)phenethyl)phenyl)pyridin-2-yl)-5-(trifluoromethyl)-1H-pyrazole-4-carboxylic acid;
1-(6-(2-(4-(3-(1H-pyrazol-1-yl)propoxy)-2-methylphenethyl)-5-fluorophenyl)pyridin-2-yl)-5-(trifluoromethyl)-1H-pyrazole-4-carboxylic acid;
1-(6-(2-(4-(3-(4-cyano-1H-pyrazol-1-yl)propoxy)-2-methylphenethyl)-5-fluorophenyl)pyridin-2-yl)-5-(trifluoromethyl)-1H-pyrazole-4-carboxylic acid;
1-(6-(5-fluoro-2-(4-(3-(4-methoxy-1H-pyrazol-1-yl)propoxy)-2-methylphenethyl)phenyl)pyridin-2-yl)-5-(trifluoromethyl)-1H-pyrazole-4-carboxylic acid;
1-(6-(2-(4-(3-(1H-1,2,4-triazol-1-yl)propoxy)-2-methylphenethyl)-5-fluorophenyl)pyridin-2-yl)-5-(trifluoromethyl)-1H-pyrazole-4-carboxylic acid;
1-(6-(2-(4-(3-(3-cyano-1H-pyrazol-1-yl)propoxy)-2-methylphenethyl)-5-fluorophenyl)pyridin-2-yl)-5-(trifluoromethyl)-1H-pyrazole-4-carboxylic acid;
Ethyl 1-(6-(2-(2-methyl-4-(4,4,4-trifluorobutoxy)phenethyl)phenyl)pyridin-2-yl)-5-(trifluoromethyl)-1H-pyrazole-4-carboxylate;
Isopropyl 1-(6-(2-(2-methyl-4-(4,4,4-trifluorobutoxy)phenethyl)phenyl)pyridin-2-yl)-5-(trifluoromethyl)-1H-pyrazole-4-carboxylate; or
1-(6-(3-chloro-2-(2-methyl-4-(4,4,4-trifluorobutoxy)phenethyl)phenyl)pyridin-2-yl)-5-(trifluoromethyl)-1H-pyrazole-4-carboxylic acid.

14. A pharmaceutical composition comprising a compound, or a pharmaceutically acceptable salt thereof, according to claim 13 and one or more pharmaceutically acceptable excipients.

15. A method for reducing elevated intraocular pressure in a mammal comprising administering a safe and effective amount of a compound, or a pharmaceutically acceptable salt thereof, according to claim 13, to a mammal in need thereof.

16. A method of treating glaucoma comprising administering a safe and effective amount of a compound, or a pharmaceutically acceptable salt thereof, according to claim 13, to a mammal in need thereof.

17. A method of treating ocular hypertension comprising administering a safe and effective amount of a compound, or a pharmaceutically acceptable salt thereof, according to claim 1, to a mammal in need thereof.

18. (canceled)

19. (canceled)

20. (canceled)
